(19) United States
(12) Patent Application Publication
(10) Pub. No.: US 2024/0101685 A1
(43) Pub. Date: Mar. 28, 2024

(54) NEAR INFRARED PHOTOIMMUNOTHERAPY (NIR-PIT) COMBINATION THERAPY TO TREAT CANCER

(71) Applicant: THE UNITED STATES OF AMERICA, as represented by the Secretary, Department of Health and Human, Baltimore, MD (US)

(72) Inventors: Hisataka Kobayashi, Laurel, MD (US); Peter Choyke, Rockville, MD (US)

(73) Assignee: THE UNITED STATES OF AMERICA, as represented by the Secretary, Department of Health and Human, Bethesda, MD (US)

(21) Appl. No.: 18/263,394

(22) PCT Filed: Jan. 28, 2022
(86) PCT No.: PCT/US2022/014448

§ 371 Date: Jul. 28, 2023

Related U.S. Application Data
(60) Provisional application No. 63/143,068, filed on Jan. 29, 2021.

(51) Int. Cl. C07K 16/28; A61K 31/7004; A61K 39/395; A61N 5/06; A61P 35/00

(52) U.S. Cl. CPC C07K 16/2863; A61K 31/7004; A61K 39/3955; A61N 5/0616; A61P 35/00; C07K 16/2818; C07K 16/2827; C07K 16/2884; A61K 2039/507; A61N 2005/0659; C07K 2317/24

(57) ABSTRACT

Provided herein are methods of treating a subject with cancer using a therapeutically effective amount of one or more one or more tumor-specific antibody-IR700 molecules. The methods can further include administering to the subject a therapeutically effective amount of (a) one or more CTLA4 antibody-IR700 molecules, one or more PD-L1 antibody-IR700 molecules, or combinations thereof, (b) one or more reducing agents, (c) one or more immunoactivators, or combinations of a, b, and c, for example, either simultaneously or substantially simultaneously with the tumor-specific antibody-IR700 molecules, or sequentially (for example, within about 0 to 24 hours). The method also includes irradiating the subject or cancer cells in the subject (for example, a tumor or cancer cells in the blood) at a wavelength of 660 to 740 nm at a dose of at least 1 J/cm². The use of one or more reducing agents can reduce edema resulting from treatment.

### CROSS REFERENCE TO RELATED APPLICATION

This application claims the benefit of the earlier filing date of U.S. Provisional Patent Application No. 63/143,068, filed on Jan. 29, 2021, the entirety of which is incorporated herein by reference.

### ACKNOWLEDGMENT OF GOVERNMENT SUPPORT

This invention was made with Government support under project number Z01 ZIA BC 011513 by the National Institutes of Health, National Cancer Institute. The Government has certain rights in the invention.

### FIELD

This disclosure relates to methods of using antibody-IR700 conjugates in combination with other therapeutic agents, such as one or more anti-CTLA4-IR700 conjugates, anti-PD-L1-IR700 conjugates, one or more immunoactivators, and/or one or more reducing agents, to kill cells, such as cancer cells, following irradiation with near infrared (NIR) light.

### BACKGROUND

Although there are several therapies for cancer, there remains a need for therapies that effectively kill the tumor cells while not harming non-cancerous cells.

In order to minimize the side effects of conventional cancer therapies, including surgery, radiation and chemotherapy, molecularly targeted cancer therapies have been developed. Among the existing targeted therapies, monoclonal antibodies (MAb) therapy have the longest history. Many therapeutic MAbs have been approved by the Food and Drug Administration (FDA) (Waldmann, Nat Med 9:269-277, 2003; Reichert et al., Nat Biotechnol 23:1073-1078, 2005). Effective MAb therapy traditionally depends on three mechanisms: antibody-dependent cellular cytotoxicity (ADCC), complement-dependent cytotoxicity (CDC), and receptor blockade, and requires multiple high doses of the MAb. MAbs have also been used at lower doses as vectors to deliver therapies such as radionuclides (Goldenberg et al., J Clin Oncol 24, 823-834, 2006) or chemical or biological toxins (Pastan et al., Nat Rev Cancer 6:559-565, 2006). Ultimately, however, dose limiting toxicity relates to the biodistribution and catabolism of the antibody conjugates.

Conventional photodynamic therapy, which combines a photosensitizing agent with the physical energy of non-ionizing light to kill cells, has been less commonly employed for cancer therapy because the currently available non-targeted photosensitizers are also taken up in normal tissues, thus, causing side effects, although the excitation light itself is harmless in the near infrared (NIR) range. Cancer immunotherapy, which includes the use of immune modulatory antibodies, cancer vaccines, and cell-based therapies, has also become a strategy in the control of cancer (Chen and Mellman, Immunity 39:1-10, 2013; Childs and Carsten, Nat. Rev. Drug Discov. 14:487-498, 2015; June et al., Sci. Transl. Med. 7:280ps7, 2015; Melero et al., Nat. Rev. Cancer 15:457-472, 2015).

Near infrared photoimmunotherapy (NIR-PIT) is a cancer treatment that employs a targeted monoclonal antibody-photo-absorber conjugate (APC). Following antibody localization of the APC to a tumor cell surface antigen, NIR light is used to induce highly selective cytolysis. NIR-PIT induces rapid, necrotic cell death that yields innate immune ligands that activate dendritic cells (DCs), consistent with immunogenic cell death (ICD). A description of how NIR-PIT kills tumor cells is described in Sato et al. (ACS Cent. Sci. 4:1559-69, 2018). Briefly, following binding of the antibody-IR700 conjugate to its target, activation by NIR light causes physical changes in the shape of antibody-antigen complexes that induce physical stress within the cellular membrane, leading to increases in transmembrane water flow that eventually lead to cell bursting and necrotic cell death. In September 2020, the first APC for human use, cetuximab-IR700 (ASP1929) was conditionally approved and registered for clinical use by the Pharmaceuticals and Medical Devices Agency (PMDA) in Japan.

Cytotoxic T-lymphocyte antigen 4 (CTLA4) is a major immune checkpoint ligand molecule mediating antitumor immune suppression. The antitumor efficacy of anti-CTLA4 immunotherapy was first reported in 1996, leading to FDA approval of the CTLA4-targeted antibody, ipilimumab, in 2011. In addition to CTLA4 targeted therapies, therapies targeting regulatory T cells (Tregs), have been shown to induce an antitumor effect. There are three key mechanisms by which Tregs normally downregulate antitumor immunity; (i) promoting the CTLA4 axis, (ii) reducing the amount of IL-2 available for effector T cells, and (iii) producing immune-suppressive cytokines, such as IL-10 and TGF-β. Indeed, anti-CTLA4 immunotherapy is thought to be effective not just because it interferes with the CTLA4 pathway, but also because it leads to depletion of intra-tumoral Tregs. However, systemic cancer immunotherapies, including anti-CTLA4 therapy and Treg depletion, often induce autoimmune adverse events; therefore, more tumor-specific methods would be desirable.

Another major immune checkpoint ligand molecule mediating antitumor immune suppression is the programed cell death ligand-1 (PD-L1), which binds to PD-1 receptor expressing on the T lymphatic cells and suppresses the cell function. PD-L1 conditionally expresses on various cells including cancer cells. Anti-tumor host immunity that relies on cytotoxic T-cells could be suppressed under a PD-L1 expressing micro-environment. Blockade of PD-1/PD-L1 axis using antibodies against these molecules inhibits the immune checkpoint as anti-CTLA4 therapies do, resulted in effective immune activation against cancer cells in some proportion of patients. However, similar to anti-CTLA4 therapy, systemic cancer immunotherapies, including anti-PD-1/PD-L1 therapy often induce autoimmune adverse events; therefore, more tumor-specific methods would be desirable.

### SUMMARY OF THE DISCLOSURE

Provided herein are methods of treating a subject with cancer with a combination of tumor-specific protein antibody-IR700 molecules (antibody-photoabsorber conjugates (APCs)) and/or one or more immunoactivators in combination with (1) one or more cytotoxic T-lymphocyte-associated protein 4 (CTLA4) antibody-IR700 molecules, one or more programmed death ligand 1 (PD-L1) antibody-IR700 molecules, or combinations thereof, (2) one or more reducing agents, or both (1) and (2), using NIR-photoimmunotherapy (PIT).

In one example, the methods include administering to a subject with cancer a therapeutically effective amount of (a) (i) one or more antibody-IR700 molecules, where the antibody specifically binds to a cancer cell surface molecule, such as a tumor-specific antigen, and/or (ii) one or more immunoactivators (such as IL-15, interferon gamma, or both), and (b) one or more CTLA4 antibody-IR700 molecules, one or more PD-L1 antibody-IR700 molecules, or both. In some examples, the tumor-specific protein comprises epidermal growth factor receptor (EGFR/HER1), mesothelin, prostate specific membrane antigen (PSMA), HER2/ERBB2, CD3, CD18, CD20, CD25 (IL-2Rα receptor), CD30, CD33, CD44, CD52, CD133, CD206, carcinoembryonic antigen (CEA), alpha-fetoprotein (AFP), Lewis Y, tumor-associated glycoprotein 72 (TAG72), vascular endothelial growth factor (VEGF), VEGF receptor (VEGFR), epithelial cell adhesion molecule (EpCAM), ephrin type-A receptor 2 (EphA2), glypican-1, glypican-2, glypican-3, gpA33, a mucin (such as MUC1, MUC4, MUC5AC, or cancer antigen 125 (CA125)), CAIX, a folate-binding protein, a ganglioside (such as GD2, GD3, GM1 or GM2), integrin αVβ3, integrin α5 β,1, Erb-B2 Receptor Tyrosine Kinase 3 (ERBB3), MET Proto-Oncogene, Receptor Tyrosine Kinase (MET), insulin like growth factor 1 receptor (IGF1R), ephrin type-A receptor 3 (EPHA3), tumor necrosis factor-related apoptosis-inducing ligand receptor 1 (TRAILR1), TRAILR2, receptor activator of nuclear factor kappa-B ligand (RANKL), fibroblast activation protein (FAP), tenascin, BCR complex, gp72, HLA-DR 10β, HLA-DR antigen, IgE, CA 242, polymorphic epithelial mucin (PEM) antigen, SK-1 antigen, programmed death 1 (PD-1), or programmed death ligand 2 (PD-L2). In a specific example, the tumor-specific protein is EGFR/HER1, and in some of those examples the antibody-IR700 molecule includes panitumumab or cetuximab. In a specific example, EGFR antibody-IR700 and CTLA4 antibody-IR700 are administered. In a specific example, IL-15 and CTLA4 antibody-IR700 are administered. In a specific example, IFN-gamma and CTLA4 antibody-IR700 are administered. In a specific example, EGFR antibody-IR700 and PD-L1 antibody-IR700 are administered. In a specific example, IL-15 and PD-L1 antibody-IR700 are administered. In a specific example, IFN-gamma and PD-L1 antibody-IR700 are administered. The molecules of (a) and (b) can be administered simultaneously, substantially simultaneously, or sequentially (for example, within about 0 to 24 hours of one another). In some examples, the molecules of (a) and (b) are administered intravenously. The subject or cancer cells in the subject (for example, a tumor, or cancer cells in the blood) are then subsequently irradiated at a wavelength of 660 to 740 nm, such as 660 to 710 nm or 680 or 690 nm, at a dose of at least 1 J/cm² (such as at least 10 J/cm² or at least 20 J/cm², such as 10 to 60 J/cm², 10 J/cm² to 50 J/cm², 20 to 50 J/cm², 20 J/cm² to 60 J/cm², ₒᵣ 20 to 30 J/cm²).

Methods are provided for treating cancer in a subject, which include administration of one or more reducing agents to reduce undesired side effects of NIR-PIT therapy, such as edema. Such methods can include administering to the subject a therapeutically effective amount of one or more antibody-IR700 molecules, wherein the antibody specifically binds to a tumor-specific protein on the surface of a cancer cell or an immune cell specific protein on the surface of an immune cell (such as a T cell). In some examples, the tumor-specific protein or immune cell specific protein is or includes HER1/EGFR, CTLA4, PD-L1, mesothelin, PSMA, HER2/ERBB2, CD3, CD18, CD20, CD25 (IL-2Rα receptor), CD30, CD33, CD44, CD52, CD133, CD206, CEA, AFP, Lewis Y, TAG72, VEGF, VEGFR, EpCAM, EphA2, glypican-1, glypican-2, glypican-3, gpA33, a mucin (such as MUC1, MUC4, MUC5AC, or cancer antigen 125 (CA125)), CAIX, a folate-binding protein, a ganglioside (such as GD2, GD3, GM1 or GM2), integrin αVβ3, ERBB3, MET, IGF1R, EPHA3, TRAILR1, TRAILR2, RANKL, FAP, tenascin, BCR complex, gp72, HLA-DR 10β, HLA-DR antigen, IgE, CA 242, PEM antigen, SK-1 antigen, PD-1, or PD-L2. In one example, the tumor-specific protein is or includes HER1/EGFR, and in some of those examples the antibody is or includes panitumumab or cetuximab. In one example, the immune cell specific protein is or includes CTLA4, and in some of those examples the antibody is or includes ipilimumab or tremelimumab. In one example, the tumor-specific protein is or includes PD-L1, and in some of those examples the antibody is or includes atezolizumab, avelumab, durvalumab, cosibelimab, KN035 (envafolimab), BMS-936559, BMS935559, MEDI-4736, MPDL-3280A, or MEDI-4737. The method further includes administering to the subject a therapeutically effective amount of one or more reducing agents, such as L-sodium ascorbate, ascorbic acid, L-cysteine, glutathione, or combinations thereof. In one example the one or more reducing agents is or includes L-sodium ascorbate. In some examples, the method further includes administering to the subject a therapeutically effective amount of one or more CTLA4 antibody-IR700 molecules, one or more PD-L1 antibody-IR700 molecules, one or more immunoactivators, or combinations thereof. The one or more tumor-specific antibody-IR700 molecules and the one or more reducing agents (and optionally the one or more CTLA4 antibody-IR700 molecules, one or more PD-L1 antibody-IR700 molecules, one or more immunoactivators, or combinations thereof) can be administered sequentially or concurrently. In some examples, antibody-IR700 molecules are administered intravenously and reducing agents are administered intraperitoneally. The method also includes irradiating the subject and/or irradiating cancer cells in the subject at a wavelength of 660 to 740 nm, such as 660 to 710 nm or 680 or 690 nm, at a dose of at least 1 J/cm² (such as at least 10 J/cm² or at least 20 J/cm², such as 10 to 60 J/cm², 10 J/cm² to 50 J/cm², 20 to 50 J/cm², 20 J/cm² to 60 J/cm², ₒᵣ 20 to 30 J/cm²). In some examples, the irradiation is performed after administration of the one or more tumor-specific antibody-IR700 molecules and the one or more reducing agents. In some examples, the one or more reducing agents are administered prior to the irradiating. In some examples, the one or more reducing agents are administered after the irradiating. Use of one or more reducing agents can reduce undesirable effects of NIR-PIT, such as edema, acute inflammatory reaction, or both. In some examples, use of one or more reducing agents in combination with NIR-PIT reduces edema in the treated subject by at least 10%, at least 20%, at least 30%, at least 40%, at least 50%, at least 60%, at least 75%, at least 80%, at least 90%, or even at least 95%, as compared to the amount of edema without use of one or more reducing agents with NIR-PIT. In some examples, use of one or more reducing agents in combination with NIR-PIT reduces acute inflammatory reaction in the treated subject by at least 10%, at least 20%, at least 30%, at least 40%, at least 50%, at least 60%, at least 75%, at least 80%, at least 90%, or even at least 95%, as compared to the amount of acute inflammatory reaction without use of one or more reducing agents with NIR-PIT.

In some examples, the methods provided herein further include selecting a subject with cancer having a tumor or cancer that expresses a cancer cell surface protein that can specifically bind to the antibody-IR700 molecule. In some examples, the cancer or cancer cell treated with the disclosed methods is a cancer or cancer cell of the breast, liver, colon, ovary, prostate, pancreas, brain, cervix, kidney, bone, skin, head and neck, oropharynx or blood. In one example, the caner or cancer cell wherein the cancer is a cancer in the airway or mediastinum. In some examples, the cancer treated is a highly or moderately immunogenic cancer, such as melanoma, lung cancer, or renal cell carcinoma. In some examples, the cancer treated is a low immunogenic cancer, such as breast cancer, pancreatic cancer, oropharyngeal cancer, or oral squamous cell cancer.

In some examples, the disclosed methods have an abscopal effect.

In some examples, the method is a method of killing a cancer cell in a subject's blood, and the irradiating step includes irradiating the cancer cell with a NIR LED at a wavelength of 660 to 740 nm at a dose of at least 1 J/cm² (for example 680 nm or 690 nm at a dose of 10 to 60 J/cm², such as a dose of 20 to 50 J/cm² or 20 to 30 J/cm²), wherein the NIR LED is present in a wearable device worn by the subject.

The irradiating step of the disclosed methods can include administering two or more doses of irradiation at a wavelength of 660 to 740 nm and at a dose of at least 1 J/cm². For example, the two or more doses of irradiation are administered within about 12 to 36 hours, such as about 24 hours, of one another.

In some examples, the subject is administered two or more doses of the one or more one or more antibody-IR700 molecules that specifically bind to the tumor-specific protein on the surface of the cancer cell, such as 2, 3, 4, 5, 6, 7, 8 9 or 10 doses. In some examples, the two or more doses of the one or more antibody-IR700 molecules that specifically bind to the tumor-specific protein on the surface of the cancer cell are separated by at least 12 hours, at least 24 hours, at last 48 hours, or at least one week, such as about 24 to 48 hours.

In some examples, the CTLA4 antibody conjugated to IR700 is or includes ipilimumab or tremelimumab.

In some examples, the PD-L1 antibody conjugated to IR700 is or includes atezolizumab, avelumab, durvalumab, cosibelimab, KN035 (envafolimab), BMS-936559, BMS935559, MEDI-4736, MPDL-3280A, or MEDI-4737. In some examples, the PD-L1 antibody conjugated to IR700 is or includes atezolizumab or avelumab. In some examples, the PD-L1 antibody conjugated to IR700 is or includes durvalumab, KN035, or cosibelimab. In some examples, the PD-L1 antibody conjugated to IR700 comprises or consists of BMS-936559, BMS935559, MEDI-4736, MPDL-3280A, or MEDI-4737.

In some examples, the EGFR antibody used in the EGFR antibody-IR700 molecule is or includes panitumumab or cetuximab.

In some examples, the subject is administered two or more doses of the CTLA4 antibody-IR700 molecules, and/or one or more PD-L1 antibody-IR700 molecules, such as 2, 3, 4, 5, 6, 7, 8 9 or 10 doses. In some examples, the two or more doses are separated by at least 12 hours, at least 24 hours, at last 48 hours, or at least one week, such as about 24 to 48 hours.

In some examples, the tumor-specific antibody binds to one or more proteins on the cancer cell surface (such as a receptor), wherein the protein on the cancer cell surface is not significantly found on non-cancer cells (such as normal healthy cells) and thus the antibody will not significantly bind to the non-cancer cells. In one example the tumor-specific protein is HER1/EGFR. In a specific example the EGFR antibody specific for EGFR is or includes panitumumab or cetuximab.

In one example the tumor-specific protein is HER2 or PSMA. In one example the tumor-specific protein is PD-L1 or CTLA4. Additional exemplary tumor-specific proteins that can be recognized by the tumor-specific antibody-IR700 molecule (e.g., include an antibody specific to one or more of these antigens) include mesothelin, PSMA, CD3, CD18, CD20, CD25 (IL-2Rα receptor), CD30, CD33, CD44, CD52, CD133, CD206, CEA, AFP, Lewis Y, TAG72, VEGF, EpCAM, EphA2, glypican-1, glypican-2, glypican-3, gpA33, a mucin (such as MUC1, MUC4, MUC5AC, or cancer antigen 125 (CA125)), CAIX, PSMA, folate-binding protein, a ganglioside (such as GD2, GD3, GM1 or GM2), VEGF receptor (VEGFR), integrin αVβ, integrin α5 β3,1, integrin αVβ3, integrin α5 β3,1, Erb-B2 ERBB3, MET, IGF1R, EPHA3, TRAILR1, TRAILR2, RANKL, FAP, tenascin, BCR complex, gp72, HLA-DR 10β, HLA-DR antigen, IgE, CA 242, polymorphic epithelial mucin (PEM) antigen, SK-1 antigen, PD-L1, and programmed death ligand 2 (PD-L2). Additional exemplary tumor-specific proteins and antibodies are provided herein (including in Table 1, below).

In some examples, the therapeutically effective amount of one or more antibody-IR700 molecules (such as EGFR antibody-IR700) is at least 100 mg/m², such as at least 200 mg/m², at least 300 mg/m², at least 400 mg/m², or at least 500 mg/m², such as 100-800 mg/m², 100-500 mg/m², 250-700 mg/m², 250-500 mg/m², or 300-500 mg/m². In some examples, the therapeutically effective amount of one or more CTLA4 antibody-IR700 molecules is at least 100 mg/m², such as at least 200 mg/m², at least 300 mg/m², at least 400 mg/m², or at least 500 mg/m², such as 100-800 mg/m², 100-500 mg/m², 250-700 mg/m², 250-500 mg/m², or 300-500 mg/m². In some examples, the therapeutically effective amount of one or more PD-L1 antibody-IR700 molecules is at least 100 mg/m², such as at least 200 mg/m², at least 300 mg/m², at least 400 mg/m², or at least 500 mg/m², such as 100-800 mg/m², 100-500 mg/m², 250-700 mg/m², 250-500 mg/m², or 300-500 mg/m². In some examples, the therapeutically effective amount of one or more antibody-IR700 molecules (such as EGFR antibody-IR700) is at least 10 mg, at least 15 mg, at least 50 mg, at least 100 mg, at least 200 mg, at least 300 mg, at least 400 mg, or at least 500 mg, such as 10-1000 mg, 15-1000 mg, 10-500 mg, 15-300 mg, or 15-100 mg. In some examples, the therapeutically effective amount of one or more CTLA4 antibody-IR700 molecules is at least 10 mg, at least 15 mg, at least 50 mg, at least 100 mg, at least 200 mg, at least 300 mg, at least 400 mg, or at least 500 mg, such as 10-1000 mg, 15-1000 mg, 10-500 mg, 15-300 mg, or 15-100 mg. In some examples, the therapeutically effective amount of one or more PD-L1 antibody-IR700 molecules is at least 10 mg, at least 15 mg, at least 50 mg, at least 100 mg, at least 200 mg, at least 300 mg, at least 400 mg, or at least 500 mg, such as 10-1000 mg, 15-1000 mg, 10-500 mg, 15-300 mg, or 15-100 mg. In some examples, the therapeutically effective amount of one or more reducing agents is at least 1 g, such as at least 5 g, at least 10 g, at least 25 g, at least 50 g, at least 100 g, at least 200 g or at least 300 g, such as 1-300 g, 10-300 g, 5 to 50 g, 5 to 25 g, 5 to 100 g, or 100-300 g.

In some examples, the cancer cells are in a subject's blood, and wherein irradiating the cancer cells comprises irradiating the blood by using a device worn by the subject, wherein the device comprises a near infrared (NIR) light emitting diode (LED).

In some examples, the method further includes selecting a subject with a cancer that expresses the tumor-specific protein that specifically binds to the antibody-IR700 molecule.

In some examples, the disclosed methods reduce the weight, volume or size of the cancer by at least 25% relative to the absence of treatment; reduce the weight, volume or size of a metastasis by at least 25% relative to the absence of treatment, wherein the metastasis is not irradiated at a wavelength of 660 to 740 nm and is located distant from the irradiated area of the tumor or lesion (e.g., there is an abscopal effect); increase survival time of the subject relative to the absence of treatment; increase progression-free survival time of the subject relative to the absence of treatment; increase disease-free survival time of the subject relative to the absence of treatment; or combinations thereof.

In some examples, the disclosed methods, for example those that utilize an CTLA4 antibody-IR700 molecule, selectively kill CD4⁺Foxp3⁺ Tregs; selectively deplete CTLA4⁺ cells from total live cell and T cell populations within the cancer but not within regional lymph nodes or the spleen; increase the CD8⁺/CD4⁺Foxp3⁺ ratio; increase the CD8+/Treg ratio; decrease intra-tumoral blood perfusion; or combinations thereof.

In some examples, the disclosed methods further include detecting the cancer cell with fluorescence lifetime imaging, for example about 0 to 48 hours after the irradiating step.

In some examples, the disclosed methods further include administering to the subject a therapeutically effective amount of one or more immunoactivators, such as IL-15, interferon gamma, or both. In particular embodiments, the immunoactivators include one or more immune system activators and/or inhibitors of immuno-suppressor cells, such as an antagonistic PD-1 antibody, antagonistic PD-L1 antibody, or CD25 antibody-IR700 molecule. In some examples, the inhibitor of immuno-suppressor cells inhibits activity and/or kills regulatory T (Treg) cells. In other examples, the immune system activator includes one or more interleukins (such as IL-2 and/or IL-15). The immunomodulator may, in some examples, increase production of memory T cells specific for one or more proteins expressed by the cancer cells. In some examples, the therapeutically effective amount of one or more immunoactivators is at least 0.1 mg, at least 0.5 mg, at least 1 mg, at least 2 mg, at least 5 mg, at least 10 mg, or at least 50 mg, such as 0.1 to 100 mg, 0.5 to 50 mg, 0.5 to 25 mg, 0.5 to 10 mg, 1 to 5 mg, 2 to 4 mg, or 1 to 10 mg (for example for iv administration), or at least 0.01 mg, at least 0.05 mg, at least 0.1 mg, or at least 1 mg, such as 0.01 to 1 mg, 0.1 to 1 mg, 0.01 to 0.075 mg, 0.5 to 1 mg, 0.1 to 0.5 mg, or 0.05 to 0.5 mg (for example for intratumoral administration).

In some examples, the one or more immunoactivators are administered prior to administration of the NIR irradiation. In some examples, the one or more immunoactivators are administered following administration of the NIR irradiation. In some examples, the one or more immunoactivators are administered prior to and following administration of the NIR irradiation.

Provided herein are methods for treating an EGFR-expressing cancer in a subject. Such methods can include administering to the subject (1) a therapeutically effective amount of one or more anti-EGFR-IR700 molecules, and (2) a therapeutically effective amount of one or more CTLA4 antibody-IR700 molecules. Following the administration, the method further includes irradiating the subject and/or irradiating and EGFR-expressing cancer cells in the subject at a wavelength of 670 to 700 nm and at a dose of 4 to 100 J/cm² (for example 680 nm or 690 nm at a dose of 10 to 60 J/cm², such as a dose of 20 to 50 J/cm² or 20 to 30 J/cm²), thereby treating the EGFR-expressing cancer in the subject. In some examples, the method further includes administering to the subject a therapeutically effective amount of one or more immunoactivators, such as IL-15, interferon gamma, or both, for example before or after the irradiation (or both). In some examples, the method further includes administering to the subject a therapeutically effective amount of one or more reducing agents, such as L-sodium ascorbate, for example before or after the irradiation (or both). The one or more anti-EGFR-IR700 molecules and the one or more CTLA4 antibody-IR700 molecules can be administered sequentially or concurrently. In some examples the EGFR antibody conjugated to IR700 includes or is panitumumab or cetuximab. In some examples the CTLA4 antibody conjugated to IR700 includes or is ipilimumab or tremelimumab.

Provided herein are methods for treating cancer in a subject. Such a method can include administering to the subject a therapeutically effective amount of one or more anti-PD-L1-IR700 molecules, and administering to the subject a therapeutically effective amount of one or more immunoactivators (such as IL-15, interferon gamma, or both) and subsequently irradiating the subject and/or irradiating cancer cells in the subject at a wavelength of 670 to 700 nm and at a dose of 4 to 100 J/cm². The one or more anti-PD-L1-IR700 molecules and the one or more immunoactivators can be administered sequentially or concurrently. In some examples the PD-L1 antibody conjugated to IR700 includes or is atezolizumab, avelumab, durvalumab, cosibelimab, KN035 (envafolimab), BMS-936559, BMS935559, MEDI-4736, MPDL-3280A, or MEDI-4737.

In some examples, the method further includes administering to the subject a therapeutically effective amount of one or more reducing agents, such as L-sodium ascorbate, for example before or after the irradiation (or both). In some examples, the method further includes administering to the subject a therapeutically effective amount of one or more antibody-IR700 molecules, wherein the antibody specifically binds to a tumor-specific protein on the surface of a cancer cell. In some example the tumor-specific protein comprises epidermal growth factor receptor HER1/EGFR, CTLA4, mesothelin, PSMA, HER2/ERBB2, CD3, CD18, CD20, CD25 (IL-2Rα receptor), CD30, CD33, CD44, CD52, CD133, CD206, CEA, AFP, Lewis Y, tumor-associated glycoprotein 72 (TAG72), VEGF, VEGFR, EpCAM, EphA2, glypican-3, gpA33, a mucin, CAIX, a folate-binding protein, a ganglioside, integrin αVβ3, ERBB3, MET, IGF1R, EPHA3, TRAILR1, TRAILR2, RANKL, FAP, tenascin, BCR complex, gp72, HLA-DR 10β, HLA-DR antigen, IgE, CA 242, PEM antigen, SK-1 antigen, PD-1, or PD-L2.

Also provided are methods for reducing edema resulting from cancer treatment in a subject, such as NIR-PIT therapy that uses one or more antibody-IR700 molecules. Such a method can include administering to the subject a therapeutically effective amount of one or more antibody-IR700 molecules, wherein the antibody specifically binds to a tumor-specific protein on the surface of a cancer cell (such as EGFR or VEGF) or an immune cell (such as CTLA4) and administering to the subject a therapeutically effective amount of a reducing agent, such as L-sodium ascorbate. The subject and/or cancer cells are irradiated at a wavelength of 660 to 740 nm and at a dose of at least 1 J/cm². The one or more antibody-IR700 molecules and the one or more reducing agents can be administered sequentially or concurrently.

The foregoing and other features of the disclosure will become more apparent from the following detailed description of several embodiments, which proceeds with reference to the accompanying figures.

### BRIEF DESCRIPTION OF THE FIGURES

FIGS. 1A-1E: Characteristics of the mEERL-hEGFR cell line and evaluation by panitumumab- or CTLA4-targeted NIR-PIT. (A) Validation of panitumumab-IR700 and CTLA4-IR700 by SDS-PAGE (left: Colloidal Blue staining, right: 700 nm fluorescence). Each diluted antibody was used as a control. (B) Membrane damage of mEERL-hEGFR cells induced by NIR-PIT was measured using PI staining. Each value represents means±SEM of independent experiments. (C) Metabolic activity measured by MTT assay. Control groups either had no APC administration or NIR light irradiation only. Each value represents the mean (% of control mean)±SEM of independent experiments. (D) Histologic evaluation with H-E staining. Panitumumab NIR-PIT induced swelling and vacuolation of the tumor cells within one hour after light exposure, while CTLA4 NIR-PIT induced no obvious changes. Scale bars represent 100 μm (top) or 50 μm (bottom). (E) Representative multiplex immunohistochemistry images of LL/2-luc, MC38-luc, and mEERL-hEGFR tumors. Top, composite images of pCK and DAPI staining; bottom, composite images of CD4, FoxP3, and CD8 staining. Intratumoral CD8⁺ T cells were counted in multiplex IHC images. Data are shown as cell count per mm². Intratumoral CD8⁺ T-cell density was significantly lower in mEERL-hEGFR tumors than in the other tumors. Intratumoral CD8b/Treg ratio was also lower in mEERL-hEGFR tumors compared with the other tumors (images; ×200; scale bar, 100 μm. n=4; one-way ANOVA followed by Tukey's test; *, P<0.05; **, P<0.01; N.S., not significant).

FIGS. 2A-2E: Depletion of CTLA4 expressed cells by dual-targeted NIR-PIT. (A) CTLA4 expression of several cells in mEERL-hEGFR tumors was analyzed by flow cytometry. Representative histograms for CTLA4 expression in tumor cells (hEGFR+CD45−), CD11b+ cells, CD8+ T cells (CD3⁺CD8+), CD4+FoxP3− T cells, and Tregs (CD3+CD4+FoxP3+) and RFI of CTLA4 are shown (n=5; one-way ANOVA followed by Tukey's test; *, P<0.05; **, P<0.01; ****, P<0.0001). (B) Representative dot plots show CTLA4 expression in live cells within mEERL-hEGFR tumors with flow cytometry three hours after each NIR-PIT. (C) The percentages of CTLA4⁺ cells among total live cells within mEERL-hEGFR tumors, tumor draining lymph nodes, and spleens. CTLA4 expressing cells within tumor after CTLA4 NIR-PIT or dual NIRPIT were significantly decreased. (D) Representative dot plots show Treg populations within mEERL-hEGFR tumors 3 hours after each NIR-PIT. (E) Scatter plots show the Treg/CD3b, nonregulatoryCD4⁺ (CD4⁺FoxP3−)/Treg, and CD8+/Treg ratios within tumors, tumor draining lymph nodes, and spleens. In CTLA4 and dual NIR-PIT groups, the Treg/CD3⁺ ratio was significantly decreased, whereas the CD4⁺/Treg and CD8⁺/Treg ratios were increased. (n=4; one-way ANOVA followed by Tukey's test; **, P<0.01; ***, P<0.001; ns, not significant).

FIGS. 3A-3C: In vivo IR700 fluorescence imaging of mEERL-hEGFR tumors after injection of panitumumab-IR700 or anti-CTLA4-IR700. IR700 fluorescence and white light images were obtained using the 700 nm fluorescence channel of the Pearl Imager (LI-COR Biosciences) and analyzed using Pearl Cam Software (LI-COR Biosciences). Serial dorsal fluorescence images of the IR700 signal were obtained before and 1/4, 1, 3, 6, 9, 12, 18, 24, 48, 72, 96 and 120 hours after intravenous injection of 50 μg of pan-IR700 or CTLA4-IR700 via tail vein. Regions of interest (ROIs) were placed on the tumor and the adjacent non-tumor region (left dorsum) as background. The mean value of fluorescence intensity was calculated for each ROI. Target-to-background ratio (TBR) was calculated from fluorescence intensity of tumor and fluorescence intensity of background by the following formula: (mean fluorescence intensity of tumor)/(mean fluorescence intensity of background). (A) Series of 700 nm fluorescence images were acquired at the indicated timepoints. (B) Quantitative analysis of mean fluorescence intensity in mEERL-hEGFR tumors is shown. (C) Quantitative analysis of TBR is shown (B, C; n=5, mean±SEM).

FIGS. 4A-4D: Efficacy of in vivo CTLA4-targeted NIR-PIT for mEERL-hEGFR tumors. (A) Treatment schedule. (B) 700 nm fluorescent imaging before and after NIR-PIT in mEERL-hEGFR tumor-bearing mouse. (A.U.; arbitrary units) (C) Tumor volume curves (n=10; mean±SEM; repeated measures two-way ANOVA followed by Tukey's test; N.S., not significant). (D) Survival curves (n=10; log-rank test with Bonferroni correction; N.S., not significant).

FIGS. 5A-5D: Efficacy of in vivo combined NIR-PIT targeting hEGFR and CTLA4. (A) Treatment schedule. (B) 700 nm fluorescent imaging before and after NIR-PIT in mEERL-hEGFR tumor-bearing mouse. The 700 nm fluorescence of the tumor decreased immediately after light exposure except for I.V. group. Yellow circles represent the locations of tumors. (C) Tumor volume curves (n=12-13; mean±SEM; repeated measures two-way ANOVA followed by Tukey's test, *p<0.05; **p<0.01; ***p<0.001, vs. combined PIT group). (D) Survival curves (n=12-13, log-rank test with Bonferroni correction, *p<0.05; **p<0.01; N.S. not significant). The panitumumab NIR-PIT, CTLA4 NIR-PIT, dual NIR-PIT cleared the tumor in 1/12, 3/12, 5/13 mice, respectively.

FIGS. 6A-6F: Efficacy of in vivo combined NIR-PIT targeting CD44 and CTLA4. (A) Treatment schedule. (B) 700 nm fluorescent imaging before and after NIR-PIT in LL/2-luc tumor-bearing mouse. 700 nm fluorescence decreased immediately after NIR light exposure except for the I.V. group. Yellow circles represent the locations of the tumors. (C) BLI before and after NIR-PIT in LL/2-luc tumor bearing mice. (D) Luciferase activity calculated from BLI (n=10-12; mean±SEM; repeated measures two-way ANOVA followed by Tukey's test; *, P<0.05; N.S., not significant; vs. dual NIR-PIT group). (E), Tumor volume curves (n=10-12; mean±SEM; repeated measures two-way ANOVA followed by Tukey's test; *, p<0.05; **, p<0.01; ***, p<0.001; N.S., not significant; vs. dual NIR-PIT group). (F) Survival curves (n=10-12, log-rank test with Bonferroni correction; *, p<0.05; **, p<0.01; N.S. not significant; vs. dual NIR-PIT group).

FIGS. 7A-7G. Dual NIR-PIT improved the immunosuppressive environment within mEERL-hEGFR tumor. (A-C) Cell populations in the regional lymph nodes were analyzed 2 days after each NIR-PIT by flow cytometry in the mEERL-hEGFR model. The expression of activation markers (CD40 (A), CD8 (B), CD96 (C)) on dendritic cells were calculated. CD86 expression in dual NIR-PIT group was significantly higher compared with all other groups. (D) Multiplex immunohistochemical staining of the tumors 7 days after NIR light exposure. Right: lymphocyte marker staining; CD8 (magenta), CD4 (green) and Foxp3 (yellow). Left: Merged images. Scale bar, 100 μm. (E) Examples of CD8+cell (magenta arrow), CD4+Foxp3− cell (green arrow) and CD4+Foxp3+ cell (yellow arrow). (F) Intratumoral CD8+ cell density was significantly higher in the dual-PIT group than the control group (n=5, one-way ANOVA followed with Tukey's test, *p <0.05). (G) Intratumoral CD8+/Treg ratio was also increased in dual NIR-PIT group compared with all other groups (n=5, one-way ANOVA followed with Tukey's test, *p<0.05).

FIG. 7H. Dual-targeted NIR-PIT activates CD8⁺ T cells in draining lymph nodes. The expression of activation marker (CD25⁺ and CD69⁺) among CD8⁺ T cells was calculated. CD25 expression was significantly increased in all NIR-PIT groups. (n=5; one-way ANOVA followed by Tukey's test; *, p<0.05; N.S., not significant; vs. I.V. group).

FIGS. 8A-8C: Dual NIR-PIT acquired long-term immunity against mEERL-hEGFR tumor. The mice that achieved complete remission by dual NIR-PIT were re-inoculated with mEERL-hEGFR tumor into the contralateral flank. (A) Treatment schedule and location of reinoculation. (B) Tumor volume curves. Control=newly inoculated tumor; reinoculation=reinoculated tumor after complete clearance by dual NIR-PIT (n=5; mean±SEM; repeated measures two-way repeated measures ANOVA followed by Turkey's test, ****p<0.00001). (C) Survival curves (log-rank test, ****p<0.0001).

FIGS. 9A-9E. Efficacy of in vivo dual NIR-PIT in bilateral tumor model. (A) Treatment schedule. (B) Diagram of NIR-light exposure. The red circle indicates where NIR light was irradiated. NIR-light was exposed only to the right sided tumor. Grey marks indicate inoculated tumors. (C) 700 nm fluorescent imaging before and after NIR-PIT in mEERL-hEGFR tumor-bearing mouse. 700 nm fluorescence decreased only on the right side after NIR light exposure, whereas 700 nm fluorescence persisted in the contralateral tumor. (D) Tumor volume curves. Tumor growth of both tumors was significantly suppressed in dual NIR-PIT group compared with I.V. group (n=10, mean±SEM, vs same side of I.V. only group; two-way ANOVA repeated measures followed by Tukey's test, ****p<0.0001) (E) Survival curves (n=10, log-rank test, **p <0.01; N.S. not significant). The dual NIR-PIT cleared the tumor in 1/10 mice).

FIGS. 10A-10B. Dual-targeted NIR-PIT impairs intratumoral blood perfusion. Blood perfusion analysis after treatment. Bovine albumin (Thermo Fisher) was conjugated with IRDye 800CW NHS ester (IR800; LI-COR Biosciences) using the same methods used for IR700. We abbreviate the conjugate as albumin-IR800. 24 hours after NIR light exposure, 50 μg of albumin-IR800 was intravenously injected. Serial dorsal 800-nm fluorescence images were obtained with the 800-nm channel of a Pearl Imager. (A) The images of perfusion based on bovine albumin-IR800 fluorescence of the tumor were taken before and 5, 10, 15 minutes after the injection. (B) Bar graphs of target-to-background ratio are shown (n=5; repeated measures two-way ANOVA followed by Tukey's test; *, P<0.05; **, P<0.01).

FIGS. 11A-11E: Synthesis of anti-PD-L1-IR700 (CD274-Ab-IR700) and cell-killing specificity of PD-L1-targeted NIR-PIT. (A) Evaluation of anti-PD-L1-IR700 by SDS-PAGE (left: Colloidal Blue staining, right: 700 nm fluorescence). Unconjugated anti-PD-L1 antibody was used as a control. (B) Evaluation of anti-CTLA4-IR700 by SEC. The APC demonstrated light absorption of 280 and 689 nm and corresponding fluorescence. Therefore, purity of the conjugates is good. (C) Flow-cytometric analysis of PD-L1 expression on cancer cell lines. Representative histograms of MC38-luc (top) and LL2-luc (bottom) are shown. (D) Flow-cytometric analysis of PD-L1 expression on cancer cell lines MC38-luc (left) and LL2-luc (right) obtained from cell culture with or without interferon gamma and in vivo tumor. Interferon gamma conditionally enhance the expression of PD-L1. (E) In vitro PD-L1 targeting NIR-PIT showed better cell killing against interferon gamma treated MC38-luc cells.

FIG. 12: Flow cytometry analysis showing PD-L1 expression in cancer cell lines before and after stimulation with 20 ng/ml or 50 ng/ml IFNgamma (IFNγ).

FIG. 13: Flow cytometry analysis showing PD-L1 expression in tumor infiltrating immune cells.

FIGS. 14A-14B: Graphs showing the effect of treating of MC38-luc tumors in vivo using anti-PD-L1-IR700. Effects on tumor volume (top graphs) and survival (bottom graphs) are shown in the presence of vehicle alone (control), anti-PD-L1-IR700 without NIR light (i.v.), or anti-PD-L1-IR700 with NIR light (PIT). (A) Treatment of original tumor, (B) treatment of reinoculated mice.

FIG. 15: Effect of LL2-luc tumor treatment using anti-PD-L1-IR700 conjugate and IL15 preconditioning in vivo. Vehicle alone (control), anti-PD-L1-IR700 without NIR light (i.v. only) anti-PD-L1-IR700 with NIR light (PIT), IL-15 only, anti-PD-L1-IR700 without NIR light+IL15 preconditioning (IL15+APC), or anti-PD-L1-IR70 with NIR light+IL15 preconditioning (IL15+PIT).

FIG. 16: Photolysis analyses of SiPc. Synthetic scheme of Pc 3, which has the same axial ligands as IR700.

FIGS. 17A-17H. L-NaAA accelerates ligand release from IR700. (A) 25 μM Pc 3 with various concentration of L-NaAA in PBS was irradiated with NIR light and imaged using 700 nm fluorescence imaging. (B) Mean 700 nm fluorescence intensity was decreased in both a NIR light- and a L-NaAA dose-dependent manner (n=4, mean±SEM). (C) Aggregation after NIR light exposure of Pc 3 with L-NaAA. Along with the change from hydrophilic to hydrophobic solubility, the cyan color of IR700 decreased, and aggregation appeared, while 700 nm fluorescence was lost in a L-NaAA dose-dependent manner. (D) 3 μM panitumumab-IR700 with various concentration of L-NaAA in PBS was irradiated with NIR light and imaged using 700 nm fluorescence imaging. (E) When more than 1 mM L-NaAA in PBS was used, mean fluorescence intensity decreased in a NIR light-dose-dependent manner (n=4, mean±SEM). (F) In 3 μM panitumumab-IR700 in PBS with any concentration of NaN₃, mean fluorescence intensity after NIR light exposure was unchanged (n=4, mean±SEM). (G) ESR spectra of samples containing IR700 (0.5 mM) and L-NaAA (10 mM) in PBS irradiated with NIR light under argon saturation conditions (left side) or in the presence of air (right side) in 5 groups as follows; (i) Control spectrum (no drug), (ii) control spectrum of a sample containing IR700 (0.5 mM) and L-NaAA (10 mM) in PBS without NIR exposure, (iii) ESR spectra with NIR light exposure under the condition of (ii), (iv) control spectrum of a sample containing IR700 (0.5 mM), L-NaAA (10 mM) and NaN3 (100 mM) in PBS without NIR exposure, (v) ESR spectra with NIR light exposure under the condition of (iv). ▴; An undesirable but negligible signal originated from the quartz ESR tube as described in Example 7. (H) Remaining percentage of IR700 (post/pre) in conditions (iii) and (v). The averaged values are represented (n=2).

FIGS. 18A-18H. L-cysteine accelerates ligand release after NIR light exposure but NaN₃ inhibits this reaction. (A) Mean 700 nm fluorescence intensity in 25 μM Pc 3 with various concentrations of L-cysteine in PBS was decreased in both a NIR light- and a L-cysteine dose-dependent manner (n=4, mean±SEM). (B) Aggregation after NIR light exposure in Pc 3 with L-cysteine mixture. Cyan color of IR700 decreased and aggregation appeared, while 700 nm fluorescence was lost in a L-cysteine dose-dependent manner. (C) 3 μM panitumumab-IR700 with various concentration of L-cysteine in PBS was irradiated with NIR light and imaged using 700 nm fluorescence imaging. Under more than 1 mM L-cysteine condition in PBS, mean 700 nm fluorescence intensity was decreased in a NIR light-dose-dependent manner (n=4, mean±SEM). (D) Under more than 1 mM NaN3 with 3 μM panitumumab-IR700 in PBS, mean 700 nm fluorescence intensity after NIR light exposure was significantly decreased (n=4, mean±SEM, vs. 0 mM NaN3; two-way ANOVA repeated measures followed by Dunnett's test; *, p<0.05; **, p<0.01; ****, p<0.0001). (E) In 3 μM panitumumab-IR700 in PBS with both 1 mM L-NaAA and L-cysteine, NaN3 did not affect mean 700 nm fluorescence intensity after NIR light exposure (n=4, mean±SEM). (F) ESR spectra obtained from samples containing IR700 (0.5 mM) and L-cysteine (10 mM) in PBS when irradiated with NIR light under argon saturation conditions. (i) Control spectrum of a sample containing IR700 (0.5 mM) and L-cysteine (10 mM) in PBS without NIR light exposure. (ii) ESR spectra obtained when the samples were irradiated with NIR light. (iii) ESR spectra shown in red color were obtained by the addition of 100 mM NaN₃ to the samples containing IR700 (0.5 mM) and L-cysteine (10 mM) in PBS and irradiating them with NIR under argon-saturated conditions. The ESR spectrum overlaid in gray color is identical to the ESR spectrum obtained in the absence of NaN3 shown in (ii). ▴; An undesirable but negligible signal originated from the quartz ESR tube. Cys; L-cysteine. (G) Remaining IR700% (post/pre) in conditions (ii) and (iii). The averaged values are represented (n=2). (H) HPLC chart of cystine and cysteine in the presence of IR700 in several conditions. When the solution of IR700 containing cysteine was irradiated under hypoxic condition, the peaks of cysteine and cystine were observed, although there was no peak of cystine without irradiation. On the other hand, in the case of aerobic condition, the peak of cysteine was almost disappeared after irradiation.

FIGS. 19A-19E. The efficacy of panitumumb NIR-PIT in A431 GFP-luc cells with reducing agents with NaN₃ added in vitro. Membrane damage of A431 GFP-luc cells induced by panitumumab NIR-PIT with L-NaAA (A) or L-cysteine (B) was measured using propidium iodide (PI) staining (n=4, mean±SEM, vs. NIR-PIT without L-NaAA or L-cysteine; one-way ANOVA followed by Dunnett's test; **, p<0.01; N.S., not significant). (C) Membrane damage of A431 GFP-luc cells by NIR-PIT with both L-NaAA and L-cysteine was measured using PI staining (n=4, mean±SEM, vs. NIR-PIT without reducing agents; one-way ANOVA followed by Dunnett's test; **, p<0.01; ***, p<0.001; N.S., not significant). Under 10 mM NaN3 condition, membrane damage of A431 GFP-luc cells induced by pani-tumumab NIR-PIT with L-NaAA (D) or L-cysteine (E) was measured by PI staining (n=4, mean±SEM; one-way ANOVA followed by Tukey's test; *, p<0.05; **, p<0.01; ***, p<0.001; ****, p<0.0001; N.S., not significant).

FIGS. 20A-20D: Membrane damage of MDAMB468 GFP-luc cells induced by panitumumab NIR-PIT with L-NaAA (A) or L-cysteine (B) was measured using propidium iodide (PI) staining (n=4; **, p<0.01; N.S., not significant; vs. NIR-PIT without L-NaAA or L-cysteine; one-way ANOVA followed by Dunnett's test). Under 10 mM NaN₃ condition, membrane damage of MDAMB468 GFP-luc cells induced by panitumumab NIR-PIT with L-NaAA (C) or L-cysteine (E) was measured by PI staining. (n=4; *, p<0.05; N.S., not significant; one-way ANOVA followed by Tukey's test). Each value represents means±SEM of independent experiments (n=4).

FIGS. 21A-21D: Cell viability was assessed by bioluminescence imaging (BLI). Cells were exposed to D-luciferin (150 mg mL⁻¹, 10 μL per well; Gold Biotechnology, St. Louis, MO) 1 hour after NIR-PIT treatment, and BLI was performed (Photon Imager; Biospace Lab, Paris, France) using M3 Vision Software (Biospace Lab). ROIs were set to include the entire well, and mean luciferase activity was measured. Under 10 mM NaN3 condition, the cell viability of A431 GFP-luc cells (A, B) and MDAMB468 GFP-luc cells (C, D) induced by panitumumab NIR-PIT with L-NaAA or L-cysteine was measured by BLI. (n=4; ****, p<0.0001; N.S., not significant; one-way ANOVA followed by Tukey's test). Each value represents means±SEM of independent experiments (n=4).

FIGS. 22A-22E: Effect of reducing agents on albumin-IR700 processing. (A) Treatment schedule. (B) The representative images of fluorescence with or without L-NaAA. (C) Mean fluorescence intensity before and after NIR light exposure with or without L-NaAA in athymic mouse (n=4, mean±SEM; repeated measures two-way ANOVA followed by Tukey's test; *, p<0.05; N.S., not significant). (E) The representative images of fluorescence with or without L-cysteine. (E) Mean fluorescence intensity before and after NIR light exposure with or without L-cysteine in athymic mouse (n=4, mean±SEM; repeated measures two-way ANOVA followed by Tukey's test; N.S., not significant).

FIGS. 23A-23C: In vivo efficacy of L-NaAA alone against MC38-luc tumor. (A) Treatment schedule. (B) BLI before (day 6) and after (days 8, 10, and 12) L-NaAA administration in MC38-luc tumor bearing mice. (C) Luciferase activity calculated from BLI (n=10, mean±SEM; repeated measures two-way ANOVA followed by Tukey's test; N.S., not significant).

FIGS. 24A-24I: The efficacy of L-NaAA for CD44-targeted NIR-PIT in a MC38-luc tumor model. (A) Treatment schedule. (B) Fluorescent imaging and (C) mean 700 nm fluorescence intensity before and after CD44-targeted NIR-PIT with or without L-NaAA in MC38-luc tumor bearing mouse (n=10, mean±SEM; paired t test; N.S., not significant). (D) BLI before (day 6) and after (days 8-12) CD44-targeted NIR-PIT in MC38-luc tumor bearing mice. (E) Luciferase activity calculated from BLI (n=10; repeated measures two-way ANOVA followed by Tukey's test; ****, P<0.0001; N.S., not significant). (F) Edema formation (red arrow) was shown 1 day after CD44-targeted NIR-PIT. (G) Body weight curve (n=10, mean±SEM; repeated measures two-way ANOVA followed by Tukey's test; N.S., not significant). (H) T2WI fat-sat MRI imaging 1 day after NIR-PIT. Red arrow shows MC38-luc subcutaneous tumor. (I) Mean high intensity area calculated from coronal T2WI fat-sat MR images (n=3, mean±SEM; unpaired t test; *, p<0.05).

FIGS. 25A-25I. In vivo efficacy of CD44-targeted NIR-PIT against LL/2-luc and MOC2-luc tumors with L-NaAA. (A) Treatment schedule. (B) Fluorescent imaging and (C) mean fluorescence intensity before and after CD44-targeted NIR-PIT with or without L-NaAA in LL/2-luc tumor bearing mouse (n=10, mean±SEM; paired t test; N.S., not significant). (D) BLI before (day 6) and after (days 8-11) CD44-targeted NIR-PIT in LL/2-luc tumor bearing mice. Luciferase activity calculated from BLI in (E) LL/2-luc and (F) MOC2-luc tumors (n=10, mean±SEM; repeated measures two-way ANOVA followed by Tukey's test; ***, P<0.001; ****, P<0.0001; N.S., not significant). (G) Edema formation (red arrow) 1 day after CD44-targeted NIR-PIT with or without L-NaAA in LL/2-luc and MOC2-luc tumors. Body weight curve in (H) LL/2-luc and (I) MOC2-luc tumor-bearing mouse. (n=10, mean±SEM; one-way ANOVA followed by Tukey's test; N.S., not significant).

FIGS. 26A-26E. In vivo efficacy of panitumumab NIR-PIT against A431 GFP-luc tumor with L-NaAA. (A) Treatment schedule. (B) Fluorescent imaging and (C) mean fluorescence intensity before and after panitumumab NIR-PIT with or without L-NaAA in A431 GFP-luc tumor bearing mouse (n=8, mean±SEM; paired t test; N.S., not significant). (D) BLI before (day 6) and after (days 8-12) panitumumab NIR-PIT in A431 GFP-luc tumor bearing mice. (E) Luciferase activity calculated from BLI (n=8, mean±SEM; repeated measures two-way ANOVA followed by Tukey's test; N.S., not significant).

FIGS. 27A-27B. Magnetic resonance imaging of edema. (A) Coronal T2WI STIR images 1 day after CD44-targeted NIR-PIT with or without L-NaAA in MC38-luc tumor. (B) Mean high intensity area calculated from coronal T2WI STIR MR images after CD44-targeted NIR-PIT (n=3, mean±SEM; unpaired t test; *, p<0.05).

FIGS. 28A-28E: Imaging and quantification of reactive oxygen specimen (ROS) after NIR-PIT with L-NaAA. To evaluate ROS after NIR-PIT with or without L-NaAA, ROS was evaluated with chemiluminescence imaging (CLI), in which, L-012 (0.5 mg mouse⁻¹; FUJIFILM Wako Chemicals U.S.A Corp, Richmond, VA) was intraperitoneally injected into mice. Photon counts were evaluated with the Photon Imager (Biospace Lab) using relative light units. ROIs were placed over the entire tumor. The counts per minute of relative light units were calculated using M3 Vision Software (Biospace Lab) and converted to the percentage based on those before NIR-PIT using the following formula: [(relative light units after treatment)/(relative light units before treatment)×100(%)]. Chemiluminescent images were continually recorded 1 and 3 hours after NIR-PIT. (A) Treatment schedule. (B) CLI before and after CD44-targeted NIR-PIT in MC38-luc tumor bearing mice. (C) Photon counts calculated from CLI in MC38-luc after CD44-targeted NIR-PIT (n=5, mean±SEM; repeated measures two-way ANOVA followed by Tukey's test; *, p<0.05). (D) BLI before and after CTLA4-targeted NIR-PIT in MC38-luc tumor bearing mice. (E) Photon counts calculated from BLI in MC38-luc after CTLA4-targeted NIR-PIT (n=5, mean±SEM; repeated measures two-way ANOVA followed by Tukey's test; *, p<0.05).

FIGS. 29A-29J: The early phase efficacy of L-NaAA for CTLA4-targeted NIR-PIT in a MC38-luc tumor model. (A) Treatment schedule. (B) Fluorescent imaging and (D) mean fluorescence intensity before and after CTLA4-targeted NIR-PIT with or without L-NaAA in MC38-luc tumor bearing mouse (n=10, mean±SEM; paired t test; N.S., not significant). (D) BLI before (day 6) and after (days 8-13) CTLA4-targeted NIR-PIT in MC38-luc tumor bearing mice. (E) Luciferase activity calculated from BLI (n=10; repeated measures two-way ANOVA followed by Tukey's test; ****, p<0.0001; N.S., not significant). (G) Edema formation (red arrow) was shown 1 day after CTLA4-targeted NIR-PIT. (G) T2WI fat-sat MRI imaging 1 day after CTLA4-targeted NIR-PIT. Red arrow shows MC38-luc subcutaneous tumor. (H) Mean high intensity area calculated from coronal T2WI fat-sat MRI images (n=3, mean±SEM; unpaired t test; **, p<0.01). (I) Body weight curve (n=10, mean±SEM; one-way ANOVA followed by Tukey's test; *, p<0.05). (J) The percentages of CTLA4^{hi} cells among total live cells 3 hours after CTLA4-targeted NIR-PIT with L-NaAA (n=4; one-way ANOVA followed by Tukey's test; *, p<0.05; N.S., not significant).

FIGS. 30A-30B: Schema indicates the proposed mechanism of ROS quenching and accelerating ligand release of IR700 by L-NaAA. (A) Energy diagram of the photo-induced ligand release reaction of IR700. IR700 receives electrons and becomes a radical anion. L-cysteine and L-NaAA act as electron donors. The ligand release reaction from the radical anion is accelerated in acidic conditions. Thus, ligand release reaction is accelerated by L-NaAA. (B) Mechanism of L-NaAA for suppressing acute edema after NIR-PIT. During NIR-PIT, ROS is also generated and caused edema. L-NaAA quenches ROS resulted in preventing acute edema, yet facilitates photo-induced ligand release.

### DETAILED DESCRIPTION OF SEVERAL EMBODIMENTS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

The sequences associated with all GenBank® Accession numbers referenced herein are incorporated by reference for the sequence available on Jan. 29, 2021.

All references herein are incorporated by reference in their entireties.

In order to facilitate review of the various embodiments of the disclosure, the following explanations of specific terms are provided:

Abscopal effect: The treatment of a tumor, such as a metastasis, in a part of the body not the direct target of the local therapy (e.g., NIR-PIT). For example, irradiation of a particular tumor with NIR light in combination with appropriate antibody-IR700 conjugate(s) can reduce the size of a different tumor (e.g., a metastasis), not irradiated with NIR light. The non-irradiated/distant tumor in some examples (for example in a human) is at least 3 inches away from the tumor treated with NIR light, such as at least 4 inches, at least 5 inches, at least 10 inches, at least 12 inches, at least 18 inches, or at least 24 inches away from the tumor treated with NIR light.

Administration: To provide or give a subject an agent, such as an antibody-IR700 molecule, reducing agent, and/or an immunomodulator, by any effective route. Exemplary routes of administration include, but are not limited to, topical, systemic or local injection (such as subcutaneous, intramuscular, intradermal, intraperitoneal, intratumoral, intraosseous, intraprostatic, and intravenous), oral, ocular, sublingual, rectal, transdermal, intranasal, vaginal, and inhalation routes. In one example, administration is intravenous. In one example, administration is intraperitoneal.

Antibody: A polypeptide ligand comprising at least a light chain or heavy chain immunoglobulin variable region which specifically recognizes and binds an epitope of an antigen, such as a tumor-specific protein or a protein specifically expressed on immune cells, such as T cells. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Antibodies, such as those in an antibody-IR700 molecule, include intact immunoglobulins and the variants and portions of antibodies, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, IL); Kuby, J., Immunology, 3^{rd} Ed., W. H. Freeman & Co., New York, 1997

Typically, a naturally occurring immunoglobulin has heavy (H) chains and light (L) chains interconnected by disulfide bonds. There are two types of light chain, lambda (□) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” (mAb) is an antibody produced by a single clone of B lymphocytes or by a cell into which the light and heavy chain genes of a single antibody have been transfected. Monoclonal antibodies are produced for instance by making hybrid antibody-forming cells from a fusion of myeloma cells with immune spleen cells. Monoclonal antibodies include humanized monoclonal antibodies. In some examples, the antibody in an antibody-IR700 molecule is an mAb, such as a humanized mAb.

A “chimeric antibody” has framework residues from one species, such as human, and CDRs (which generally confer antigen binding) from another species, such as a murine antibody that specifically binds mesothelin.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. All parts of a human immunoglobulin are substantially identical to corresponding parts of natural human immunoglobulin sequences.

“Specifically binds” refers to the ability of individual antibodies to specifically immunoreact with an antigen, such as a tumor-specific antigen, relative to binding to unrelated proteins, such as non-tumor proteins, for example β-actin. For example, an EGFR-specific binding agent binds substantially only the EGFR protein in vitro or in vivo. As used herein, the term “tumor-specific binding agent” includes tumor-specific antibodies (and fragments thereof) and other agents that bind substantially only to a tumor-specific protein in that preparation.

The binding is a non-random binding reaction between an antibody molecule and an antigenic determinant of the T cell surface molecule. The desired binding specificity is typically determined from the reference point of the ability of the antibody to differentially bind the T cell surface molecule and an unrelated antigen, and therefore distinguish between two different antigens, particularly where the two antigens have unique epitopes. An antibody that specifically binds to a particular epitope is referred to as a “specific antibody.”

In some examples, an antibody (such as one in an antibody-IR700 molecule) specifically binds to a target (such as a cell surface protein, such as a tumor specific protein) with a binding constant that is at least 10³ M⁻¹ greater, 10⁴ M⁻¹ greater or 10⁵ M⁻¹ greater than a binding constant for other molecules in a sample or subject. In some examples, an antibody (e.g., mAb) or fragments thereof, has an equilibrium constant (Kd) of 1 nM or less. For example, an antibody binds to a target, such as tumor-specific protein with a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸ M, or at least about 2.0×10⁻⁸ M. Kd values can, for example, be determined by competitive ELISA (enzyme-linked immunosorbent assay) or using a surface-plasmon resonance device such as the Biacore T100, which is available from Biacore, Inc., Piscataway, NJ.

Antibody-IR700 molecule or antibody-IR700 conjugate: A molecule that includes both an antibody, such as a tumor-specific antibody, conjugated to IR700. In some examples the antibody is a humanized antibody (such as a humanized mAb) that specifically binds to a surface protein on a cancer cell, such as a tumor-specific antigen.

Antigen (Ag): A compound, composition, or substance that can stimulate the production of antibodies or a T cell response in an animal, including compositions (such as one that includes a tumor-specific protein) that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. In some examples, an antigen includes a tumor-specific protein or peptide (such as one found on the surface of a cell, such as a cancer cell) or immunogenic fragment thereof. In some examples, an antigen includes an immune cell-specific protein or peptide (such as one found on the surface of an immune cell, such as a T cell, such as a regulatory T cell) or immunogenic fragment thereof.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. In one example, the cell killed by the disclosed methods is a cancer cell.

CD25 (IL-2 receptor alpha chain): (e.g., OMIM 147730) A type I transmembrane protein present on activated T cells, activated B cells, some thymocytes, myeloid precursors, and oligodendrocytes. CD25 has been used as a marker to identify CD4+FoxP3+ regulatory T cells in mice. CD25 is found on the surface of some cancer cells, including B-cell neoplasms, some acute nonlymphocytic leukemias, neuroblastomas, mastocytosis and tumor infiltrating lymphocytes. It functions as the receptor for HTLV-1 and is consequently expressed on neoplastic cells in adult T cell lymphoma/leukemia. Exemplary CD25 sequences can be found on the GenBank® database (e.g., Accession Nos. CAA44297.1, NP_000408.1, and NP_001295171.1). Exemplary mAbs specific for CD25 are daclizumab and basiliximab, which can be attached to IR700, forming daclizumab-IR700 or basiliximab-IR700, which can be used in the disclosed methods to target CD25-expressing cancer cells, or used as an immunomodulatory molecule (e.g., to reduce tumor-infiltrating Treg cells within the tumor).

Contacting: Placement in direct physical association, including both a solid and liquid form. Contacting can occur in vitro, for example, with isolated cells, such as tumor cells, or in vivo by administering to a subject (such as a subject with a tumor, such as cancer).

Cytotoxic T-lymphocyte antigen 4 (CTLA4): (e.g., OMIM 123890) A major immune checkpoint molecule mediating antitumor immune suppression. CTLA4 is constitutively expressed in regulatory T cells but only upregulated in conventional T cells after activation—a phenomenon which is particularly notable in cancers. Exemplary CTLA4 sequences can be found on the GenBank® database (e.g., Accession Nos. AAL07473.1, AAF01489.1, and AF414120.1). An exemplary mAb specific for CTLA4 is ipilimumab, which can be attached to IR700, forming ipilimumab-IR700, which can be used in the disclosed methods to target CTLA4-expressing cells, such as melanoma CTLA4-expressing cells or CTLA4-expressing T cells.

Decrease: To reduce the quality, amount, or strength of something. In one example, a therapeutic composition that includes one or more antibody-IR700 molecules decreases the viability of cells to which the antibody-IR700 molecule specifically binds, following irradiation of the cells with NIR (for example at a wavelength of about 680 nm or 690 nm) at a dose of at least 1 J/cm² (such as 4 to 100 J/cm²), for example as compared to the response in the absence of the antibody-IR700 molecule. In some examples such a decrease is evidenced by the killing of the cells, such as cancer cells or CD4⁺Foxp3⁺ Tregs in a tumor bed. In some examples, the decrease in the viability of cells is at least 20%, at least 50%, at least 75%, or even at least 90%, relative to the viability observed with a composition that does not include an antibody-IR700 molecule. In other examples, decreases are expressed as a fold change, such as a decrease in the cell viability by at least 2-fold, at least 3-fold, at least 4-fold, at least 5-fold, at least 8-fold, at least 10-fold, or even at least 15 or 20-fold, relative to the viability observed with a composition that does not include an antibody-IR700 molecule. Such decreases can be measured using the methods disclosed herein.

Epidermal growth factor receptor (EGFR): (e.g., OMIM 131550) A transmembrane protein that is a receptor for members of the epidermal growth factor family (EGF family) of extracellular protein ligands. The epidermal growth factor receptor is a member of the ErbB family of receptors, a subfamily of four closely related receptor tyrosine kinases: EGFR (ErbB-1), HER2/neu (ErbB-2), Her 3 (ErbB-3) and Her 4 (ErbB-4). EGFR can form heterodimers with HER2 and other HER family members. Mutations affecting EGFR expression or activity can result in cancer, including adenocarcinoma of the lung, anal cancers, glioblastoma and epithelian tumors of the head and neck. Exemplary EGFR sequences can be found on the GenBank® database (e.g., Accession Nos. NP_001333826.1, CAA55587.1, and AAF14008.1). An exemplary mAb specific for EGFR is panitumumab, which can be attached to IR700, forming panitumumab-IR700, which can be used in the disclosed methods to target EGFR-expressing cells.

Immunomodulator: An immunomodulator is a substance that alters (for example, increases or decreases) one or more functions of the immune system. In some examples, an immunomodulator activates the immune system. In other examples, an immunomodulator inhibits activity of (or kills) immuno-suppressor cells. In some examples, an immunomodulator is mAb, which can be attached to IR700, forming mAb-IR700, which can be used in the disclosed methods to target cells expressing a protein recognized by the mAb. Exemplary immunomodulators include the immunoactivators IL-15 and IFN-gamma.

IR700 (IRDye® 700DX): A dye having the following formula:

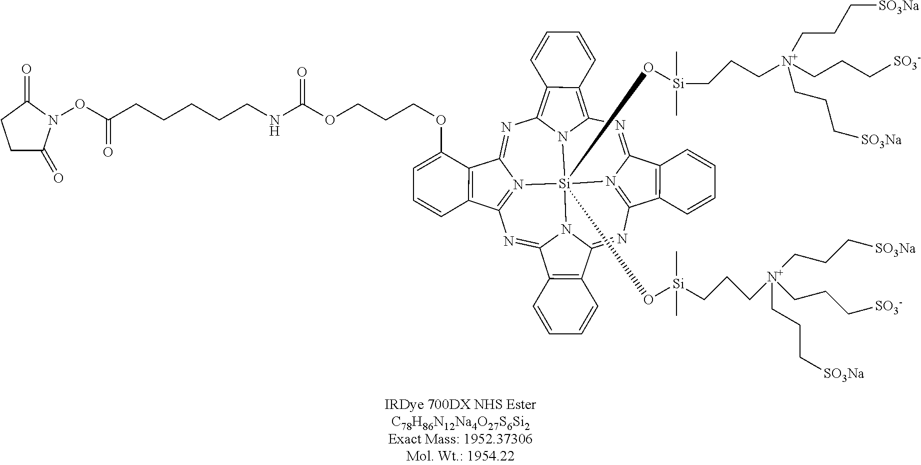

Commercially available from LI-COR (Lincoln, NE). Amino-reactive IR700 is a relatively hydrophilic dye and can be covalently conjugated with an antibody using the NHS ester of IR700. IR700 also has more than 5-fold higher extinction coefficient (2.1×10⁵ M⁻¹ cm⁻¹ at the absorption maximum of 689 nm), than conventional photosensitizers such as the hematoporphyrin derivative Photofrin® (1.2×10³M⁻¹ cm⁻¹ at 630 nm), meta-tetrahydroxyphenylchlorin; Foscan® (2.2×10⁴ M⁻¹ cm⁻¹ at 652 nm), and mono-L-aspartylchlorin e6; NPe6/Laserphyrin® (4.0×10⁴ M⁻¹cm¹ at 654 nm).

Pharmaceutical composition: A chemical compound or composition capable of inducing a desired therapeutic or prophylactic effect when properly administered to a subject. A pharmaceutical composition can include a therapeutic agent, such as one or more antibody-IR700 molecules, reducing agents, and/or one or more immunoactivators. A therapeutic or pharmaceutical agent is one that alone or together with additional compound(s) induces the desired response (such as inducing a therapeutic or prophylactic effect when administered to a subject). In a particular example, a pharmaceutical composition includes a therapeutically effective amount of at least one antibody-IR700 molecule.

Pharmaceutically acceptable vehicles: The pharmaceutically acceptable carriers (vehicles) useful in this disclosure are conventional. Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compounds, such as one or more antibody-IR700 molecules, one or more reducing agents, and/or one or more immunoactivators.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Photoimmunotherapy (PIT): A molecularly targeted therapeutic that utilizes a target-specific photosensitizer based on a near infrared (NIR) phthalocyanine dye, IRDye700DX (IR700), conjugated to monoclonal antibodies (MAb) targeting a cellular protein (such as one on the cell surface). In one example the cell surface protein is one found specifically on cancer cells or immune cells, and thus PIT can be used to kill such cells. Cell death occurs when the antibody-IR700 conjugate binds to the cells and the cells are irradiated with NIR, while cells that do not express the cell surface protein recognized the antibody-IR700 molecule are not killed in significant numbers.

Programmed death 1 (PD-1): (e.g., OMIM 600244) A type 1 membrane protein on the surface of cells that has a role in regulating the immune system's response to the cells of the human body by down-regulating the immune system and promoting self-tolerance by suppressing T cell inflammatory activity. PD-1 binds to two ligands, PD-L1 and PD-L2. Exemplary PD-1 sequences can be found on the GenBank® database (e.g., Accession Nos. CAA48113.1, NP_005009.2, and NP_001076975.1). Exemplary antagonistic mAbs specific for PD-1, which can be used in combination with the disclosed methods, include JTX-4014 by Jounce Therapeutics, nivolumab, pembrolizumab, pidilizumab, cemiplimab, spartalizumab (PDR001), camrelizumab (SHR1210), sintilimab (IBI308), tislelizumab (BGB-A317), toripalimab (JS 001, dostarlimab (TSR-042, WBP-285), INCMGA00012 (MGA012), AMP-224, and AMP-514. An exemplary mAb specific for PD-1 is nivolumab, which can be used in combination with the disclosed methods.

Programmed death ligand 1 (PD-L1): (e.g., OMIM 605402) A type 1 membrane protein on the surface of cells that suppresses the adaptive arm of immune system during particular events such as pregnancy, tissue allografts, autoimmune disease and hepatitis. The binding of PD-L1 to the inhibitory checkpoint molecule PD-1 transmits an inhibitory signal based on interaction with phosphatases (SHP-1 or SHP-2) via Immunoreceptor Tyrosine-Based Switch Motif (ITSM) motif. PD-L1 binds to PD-1, found on activated T cells, B cells, and myeloid cells, to modulate activation or inhibition. Exemplary PD-L1 sequences can be found on the GenBank® database (e.g., Accession Nos. ADK70950.1, NP_054862.1, and NP_001156884.1). Antibodies that antagonize PD-L1 activity can be conjugated to IR700 (forming anti-PD-L1-IR700) used the methods provided herein, for example in combination with a tumor-specific antigen Ab-IR700 molecule. Exemplary antagonistic mAbs specific for PD-L1 include atezolizumab, avelumab, durvalumab, cosibelimab, KN035 (envafolimab), BMS-936559, BMS935559, MEDI-4736, MPDL-3280A, and MEDI-4737, any of which can be conjugated to IR700. For example, one exemplary mAb specific for PD-L1 is durvalumab, which can be attached to IR700, forming durvalumab-IR700, which can be used in the disclosed methods to target PD-L1-expressing cells.

Reducing Agent: An element or compound that loses (or “donates”) an electron to an electron recipient (oxidizing agent) in a redox chemical reaction. A reducing agent is thus oxidized when it loses electrons in the redox reaction. Reducing agents “reduce” (or, are “oxidized” by) oxidizing agents. Thus, one or more reducing agents can be used in combination with NIR-PIT in vitro and in vivo to reduce undesired non-specific tissue damage due to reactive oxygen species generated from IR700 following exposure to NIR light. For example, reducing agents can inactivate reactive oxygen species not bound or in the area of a tumor, and reduce undesired acute inflammation in other areas of the body. Exemplary reducing agents that can be used in the methods provided herein include L-cysteine, L-sodium ascorbate (L-NaAA), ascorbic acid (such as L- or R-ascorbic acid) and glutathione. In some examples, sodium azide is not used in vivo due to its toxicity. Thus, in some examples, the reducing agent used in the disclosed methods is not sodium azide. In one example, the reducing agent used in the disclosed methods is L-NaAA.

Subject or patient: A term that includes human and non-human mammals. In one example, the subject is a human or veterinary subject, such as a mouse, rat, dog, cat, or non-human primate. In some examples, the subject is a mammal (such as a human) who has cancer, or is being treated for cancer.

Therapeutically effective amount: An amount of a composition that alone, or together with an additional therapeutic agent(s) (such as an anti-CTLA4-IR700 molecule, a anti-PD-L1-IR700 molecule, immunomodulator, and/or reducing agent) sufficient to achieve a desired effect in a subject, or in a cell, being treated with the agent. The effective amount of the agent (such as an antibody-IR700 molecule, alone or in combination with other agents) can be dependent on several factors, including, but not limited to the subject or cells being treated, the particular therapeutic agent, and the manner of administration of the therapeutic composition. In one example, a therapeutically effective amount or concentration of an antibody-IR700 molecule and/or immunomodulator is one that is sufficient to prevent advancement (such as metastasis), delay progression, or to cause regression of a disease, or which is capable of reducing symptoms caused by the disease, such as cancer. In one example, a therapeutically effective amount or concentration of an antibody-IR700 molecule and/or immunomodulator is one that is sufficient to increase the survival time of a patient with a tumor. In one example, a therapeutically effective amount or concentration of a reducing agent is one that is sufficient to decrease edema, acute inflammatory reaction, or both, in a subject treated with NIR-PIT.

In one example, a desired response is to reduce or inhibit one or more symptoms associated with cancer. The one or more symptoms do not have to be completely eliminated for the composition to be effective. For example, treatment with a modality provided herein in some examples decreases the size of a tumor (such as the volume or weight of a tumor or metastasis of a tumor), for example by at least 20%, at least 50%, at least 80%, at least 90%, at least 95%, at least 98%, or even at least 100%, as compared to the tumor size in the absence of the treatment (or with only a subset of the therapy provided herein). In one particular example, treatment with a modality provided herein kills a population of cells (such as cancer cells), for example by killing at least 20%, at least 50%, at least 60%, at least 70%, at least 80%, at least 90%, at least 95%, at least 98%, or even at least 100% of the cells, as compared to the cell killing in the absence of the treatment (or with only a subset of the therapy provided herein). In one particular example, treatment with a modality provided herein increases the survival time of a patient with a tumor (or who has had a tumor recently removed), for example increases survival by at least 20%, at least 50%, at least 60%, at least 70%, at least 80%, at least 90%, at least 95%, at least 98%, at least 100%, at least 200%, or at least 500%, as compared to the survival time in the absence of the treatment (or with only a subset of the therapy provided herein). In some examples, treatment with a modality provided herein increases an amount of memory T cells in a subject, for example an increase of at least 20%, at least 50%, at least 60%, at least 70%, at least 80%, at least 90%, at least 95%, at least 98%, at least 100%, at least 200%, or at least 500%, as compared to an amount of memory T cells in the absence of the treatment (or with only a subset of the therapy provided herein). In some examples, treatment with a modality provided herein increases an amount of polyclonal antigen-specific TIC responses against MHC type I-restricted tumor specific antigens, in a subject, for example increase by at least 20%, at least 50%, at least 60%, at least 70%, at least 80%, at least 90%, at least 95%, at least 98%, at least 100%, at least 200%, or at least 500%, as compared to an amount of polyclonal antigen-specific TIC responses against MHC type I-restricted tumor specific antigens in the absence of treatment (or with only a subset of the therapy provided herein). In some examples, treatment with a modality provided herein decreases an amount of Tregs (such as FOXP3⁺CD25⁺CD4⁺ Treg cells) in a targeted tumor, for example decrease of at least 20%, at least 50%, at least 60%, at least 70%, at least 80%, at least 90%, at least 95%, at least 98%, at least 100%, as compared to an amount of Tregs in the targeted tumor in the absence of treatment (or with only a subset of the therapy provided herein). In some examples, treatment with a modality provided herein increases the CD8+/Treg ratio in a treated subject, for example increase by at least 20%, at least 50%, at least 60%, at least 70%, at least 80%, at least 90%, at least 95%, at least 98%, at least 100%, at least 200%, or at least 500%, as compared to the CD8+/Treg ratio in the absence of treatment (or with only a subset of the therapy provided herein). In some examples, combinations of these effects are archived by the disclosed methods.

The effective amount of a therapeutic agent administered to a human or veterinary subject can vary depending upon a number of factors associated with that subject, for example the overall health of the subject. An effective amount of an agent can be determined by varying the dosage of the composition(s) and measuring the resulting therapeutic response, such as the regression of a tumor. Effective amounts also can be determined through various in vitro, in vivo or in situ immunoassays. The disclosed agents can be administered in a single dose, or in several doses, as needed to obtain the desired response. However, the effective amount can be dependent on the treatment being applied, the subject being treated, the severity and type of the condition being treated, and the manner of administration.

In particular examples, a therapeutically effective dose of an antibody-IR700 molecule is at least 0.5 milligram per 60 kilogram (mg/kg), at least 5 mg/60 kg, at least 10 mg/60 kg, at least 20 mg/60 kg, at least 30 mg/60 kg, at least 50 mg/60 kg, for example 0.5 to 50 mg/60 kg, such as a dose of 1 mg/60 kg, 2 mg/60 kg, 5 mg/60 kg, 20 mg/60 kg, or 50 mg/60 kg, for example when administered iv. In another example, a therapeutically effective dose of an antibody-IR700 molecule is at least 10 μg/kg, such as at least 100 μg/kg, at least 500 μg/kg, or at least 500 μg/kg, for example 10 μg/kg to 1000 μg/kg, such as a dose of 100 μg/kg, 250 μg/kg, about 500 μg/kg, 750 μg/kg, or 1000 μg/kg, for example when administered intratumorally or ip. In one example, a therapeutically effective dose of an antibody-IR700 molecule is at least 1 μg/ml, such as at least 500 μg/ml, such as between 20 μg/ml to 100 μg/ml, such as 10 μg/ml, 20 μg/ml, 30 μg/ml, 40 μg/ml, 50 μg/ml, 60 μg/ml, 70 μg/ml, 80 μg/ml, 90 μg/ml or 100 μg/ml administered in topical solution. However, one skilled in the art will recognize that higher or lower dosages also could be used, for example depending on the particular antibody-IR700 molecule. In particular examples, such daily dosages are administered in one or more divided doses (such as 2, 3, or 4 doses) or in a single formulation. The disclosed antibody-IR700 molecules can be administered alone, in the presence of a pharmaceutically acceptable carrier, in the presence of other therapeutic agents (such as other anti-neoplastic agents).

Generally a suitable dose of irradiation following administration of one or more antibody-IR700 molecules (and in some examples also a reducing agent) is at least 1 J/cm² at a wavelength of 660-740 nm, for example, at least 10 J/cm² at a wavelength of 660-740 nm, at least 20 J/cm² at a wavelength of 660-740 nm, at least 25 J/cm² at a wavelength of 660-740 nm, at least 50 J/cm² at a wavelength of 660-740 nm, or at least 100 J/cm² at a wavelength of 660-740 nm, for example 1 to 500 J/cm² at a wavelength of 660-740 nm. In some examples the wavelength is 660-710 nm. In specific examples, a suitable dose of irradiation following administration of the antibody-IR700 molecule is at least 1 J/cm² at a wavelength of 680 or 690 nm for example, at least 10 J/cm² at a wavelength of 680 or 690 nm, at least 20 J/cm² at a wavelength of 680 or 690 nm, at least 25 J/cm² at a wavelength of 680 or 690 nm, at least 50 J/cm² at a wavelength of 680 or 690 nm, or at least 100 J/cm² at a wavelength of 680 or 690 nm, for example 4 to 50 J/cm² or 10 to 25 J/cm² at a wavelength of 680 or 690 nm. In particular examples, multiple irradiations are performed (such as at least 2, at least 3, or at least 4 irradiations, such as 2, 3, 4, 5, 6, 7, 8, 9 or 10 separate administrations), following administration of one or more antibody-IR700 molecules (and in some examples also following administration of an immunomodulator and/or a reducing agent).

Treating: A term when used to refer to the treatment of a cell or tissue with a therapeutic agent, includes contacting or incubating one or more agents (such as one or more antibody-IR700 molecules and in some examples also one or more immunoactivators and/or one or more reducing agents) with the cell or tissue and/or administering one or more agents to a subject, for example a subject with cancer. A treated cell is a cell that has been contacted with a desired composition in an amount and under conditions sufficient for the desired response. In one example, a treated cell is a cell that has been exposed to an antibody-IR700 molecule under conditions sufficient for the antibody to bind to a surface protein on the cell, optionally contacted with a reducing agent, and irradiated with NIR light, until sufficient cell killing is achieved. In other examples, a treated subject is a subject that has been administered one or more antibody-IR700 molecules under conditions sufficient for the antibody to bind to a surface protein on the cell, optionally administered one or more reducing agents, and irradiated with NIR light, until sufficient cell killing is achieved.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Tumors include original (primary) tumors, recurrent tumors, and metastases (secondary) tumors. A tumor recurrence is the return of a tumor, at the same site as the original (primary) tumor, for example, after the tumor has been removed surgically, by drug or other treatment, or has otherwise disappeared. A metastasis is the spread of a tumor from one part of the body to another. Tumors formed from cells that have spread are called secondary tumors and contain cells that are like those in the original (primary) tumor. There can be a recurrence of either a primary tumor or a metastasis Exemplary tumors, such as cancers, that can be treated with the disclosed methods include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, head and neck cancers (e.g., adenocarcinoma, squamous cell carcinoma, metastatic squamous, such as cancers caused by HPV or Epstein-Barr virus, such as HPV16; can include cancers of the mouth, tongue, cheek, nasopharynx, throat, hypopharynx, oropharynx, larynx, and trachea), stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the tumor is an adenocarcinoma. In one example, the tumor is an oropharyngeal cancer. In one example the tumor is a squamous carcinoma, such as one of the head and neck or skin.

The methods can also be used to treat liquid tumors (e.g., hematological malignancies), such as a lymphatic, white blood cell, or other type of leukemia. In a specific example, the tumor treated is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), lymphomas (such as Hodgkin's lymphoma and non-Hodgkin's lymphoma), and myelomas.

Under conditions sufficient for: A phrase that is used to describe any environment that permits the desired activity. In one example, “under conditions sufficient for” includes administering an antibody-IR700 molecule to a subject sufficient to allow the antibody-IR700 molecule to bind to its targeted cell surface protein (such as a tumor-specific antigen or immune cell specific antigen). In particular examples, the desired activity is killing the cells to which the antibody-IR700 molecule is bound, following therapeutic irradiation of the cells.

Untreated: An untreated cell is a cell that has not been contacted with a therapeutic agent, such as an antibody-IR700 molecule and/or irradiation. In an example, an untreated cell is a cell that receives the vehicle in which the therapeutic agent(s) was delivered. Similarly, an untreated subject is a subject that has not been administered a therapeutic agent, such as an antibody-IR700 molecule and/or irradiation. In an example, an untreated subject is a subject that receives the vehicle in which the therapeutic agent(s) was delivered.

Disclosure of certain specific examples is not meant to exclude other embodiments. In addition, any treatments described herein are not necessarily exclusive of other treatment, but can be combined with other bioactive agents or treatment modalities.

### Overview of Technology

Near infrared photoimmunotherapy (NIR-PIT) is a highly-selective cancer treatment that induces necrotic/immunogenic cell death, utilizing a monoclonal antibody (mAb) conjugated to a photo-absorbing silicon-phthalocyanine dye derivative called IRDye700DX (IR700) and NIR light. Approximately 1 day after intravenous administration, antibody-photoabsorber conjugates (APCs, also referred to herein as antibody-IR700 molecules) bind to cancer cell surface markers. Application of NIR light following binding of the APCs results in ligand release on dye, dramatic changes in solubility of the APC-antigen complex and rapid, irreversible cell membrane damage of cancer cells in a highly selective manner, resulting in a highly immunogenic cell death. Clinically, this process results in edema after treatment mediated by reactive oxygen species (ROS).

CTLA4 is a major immune checkpoint molecule mediating antitumor immune suppression. Data is provided herein showing synergistic effects on tumor treatment using CTLA4-targeted NIR-PIT (which depletes intra-tumoral CTLA4⁺ cells, mostly regulatory T cells (Tregs)) in combination with tumor-targeted NIR-PIT (e.g., using a tumor-specific antibody conjugated to IR700). Thus, CTLA4-targeted NIR-PIT can effectively treat tumors by blocking CTLA4-axis as well as eliminating CTLA4-expressing immune suppressor cells, resulting in inducing potential T cell mediated antitumor immunity. Local CTLA4-expressing cell depletion in tumor beds using NIR-PIT, for example in combination with a tumor-specific antigen antibody-IR700 conjugate, can be used for treating a variety of tumor types.

Data is also provided demonstrating that use of anti-PD-L1-IR700 with NIR-PIT, can stand as a cancer+immune therapy because anti-PD-L1-IR700 targets cancer cells and blocks the PD1-PDL1 axis of immunosuppression that cured some mouse patients. Additionally, further immunoactivation improved complete response (CR) rate, for example in combination with IL-15, interferon gamma, or both, can be used to treat tumors and increase survival.

To minimize transient acute inflammatory edema without compromising therapeutic effects, reducing agents such as L-sodium ascorbate (L-NaAA) or L-cysteine were administered to quench harmful ROS and accelerate the ligand release reaction. L-NaAA and L-cysteine suppressed acute edema by reducing ROS after NIR-PIT, yet did not alter the therapeutic effects. NIR-PIT could be performed safely under existence of L-NaAA or L-cysteine without side-effects caused by unnecessary ROS production. Thus, reducing agents, such as L-cysteine and L-sodium ascorbate, can be used in combination with NIR-PIT to reduce undesired non-specific tissue damage due to reactive oxygen species, such as edema (such as transient acute edema) and acute inflammatory reaction. In some examples, the reducing agent quenches ROS, but is an electron-donor which promotes NIR-light induced ligand release reaction of IR700. In one example, the treated tumor is located near a critical structure such as the airway in the neck or the mediastinum, where edema can cause side-effects such as airway obstruction and coughing, can be treated with the disclosed methods.

Based on these observations, provided herein are methods of treating cancer in a subject using NIR-PIT, for example administration of (1) one or more CTLA4 antibody-IR700 molecules, one or more PD-L1 antibody-IR700 molecules, or combinations thereof, (2) one or more tumor-specific antibody-IR700 conjugates, one or more immunoactivators, or both, and optionally (3) one or more reducing agents. In some examples, the method includes treating cancer in a subject using NIR-PIT, for example administration of (1) one or more tumor-specific antibody-IR700 conjugates, (2) one or more reducing agents, and optionally (3) one or more immunoactivators. In some examples, the method includes treating an EGFR-expressing cancer in a subject using NIR-PIT, for example administration of (1) one or more CTLA4 antibody-IR700 molecules, (2) one or more EGFR antibody-IR700 conjugates, and optionally (3) one or more reducing agents, one or more immunoactivators, or both. In some examples, the method includes treating cancer in a subject using NIR-PIT, for example administration of (1) one or more PD-L1 antibody-IR700 molecules, (2) one or more immunoactivators, and optionally (3) one or more reducing agents, one or more tumor-specific antibody-IR700 conjugates, or both.

Such methods kill cancer cells at the site of irradiation with NIR light as well as distant non-irradiated metastasizes. The presence of reducing agents can remove or reduce reactive oxygen species that result from the irradiation of the IR700 molecules, which reduces undesired inflammation and edema. Such methods provide a highly effective treatment of various cancers, both locally and even in distant metastases away from the treated site (absopal effects).

### Methods for Treating Cancer

The present disclosure provides methods for treating a subject with cancer, such as a subject with a solid tumor or a hematological malignancy. The methods include administering to the subject an antibody that is conjugated to the dye IR700 (referred to herein as an antibody-IR700 molecule or antibody-photoabsorber conjugate (APC)), wherein the antibody in some examples specifically binds to a cancer (e.g., tumor) cell surface protein (also referred to herein as a tumor-specific antigen or protein). Tumor specific proteins are proteins that are unique to cancer cells or are much more abundant on them, as compared to other cells, such as normal cells. For example, HER2 is generally found in breast cancers, while HER1 is typically found in adenocarcinomas, which can be found in many organs, such as the pancreas, breast, prostate and colon. In some examples the antibody of the APC specifically binds to a protein on an immune cell, such as a T cell, such as a regulatory cell. In one example, the immune cell-specific protein that specifically binds to the antibody is CTLA4. The methods can further include (a) administering to the subject a therapeutically effective amount of one or more immunoactivators (such as IL-15 or IFN-gamma) (b) administering to the subject a therapeutically effective amount of one or more reducing agents (for example to reduce edema), or both (a) and (b).

The subject is administered a therapeutically effective amount of one or more tumor-specific antibody-IR700 molecules (for example in the presence of a pharmaceutically acceptable carrier, such as a pharmaceutically and physiologically acceptable fluid), under conditions that permit the antibody to specifically bind to the cancer cell surface protein. For example, the antibody-IR700 molecule can be present in a pharmaceutically effective carrier, such as water, physiological saline, balanced salt solutions (such as PBS/EDTA), aqueous dextrose, sesame oil, glycerol, ethanol, combinations thereof, or the like, as a vehicle. The carrier and composition can be sterile, and the formulation suits the mode of administration. In a specific example, the antibody-IR700 molecule is EGFR antibody-IR700. In some examples, the tumor-specific antibody is specific for HER1/EGFR, PD-L1, mesothelin, PSMA, HER2/ERBB2, CD3, CD18, CD20, CD25 (IL-2Rα receptor), CD30, CD33, CD44, CD52, CD133, CD206, CEA, AFP, Lewis Y, TAG7), VEGF, VEGFR, EpCAM, EphA2, glypican-1, glypican-2, glypican-3, gpA33, a mucin, CAIX, a folate-binding protein, a ganglioside, integrin αVβ, ERBB3, MET, IGF1R, EPHA3, TRAILR1, TRAILR2, RANKL, FAP, tenascin, BCR complex, gp72, HLA-DR 10β, HLA-DR antigen, IgE, CA 242, PEM antigen, SK-1 antigen, PD-1, or PD-L2.

In some examples, the subject is further administered a therapeutically effective amount of one or more CTLA4 antibody-IR700 molecules, one or more PD-L1 antibody-IR700 molecules, or combinations thereof (for example in the presence of a pharmaceutically acceptable carrier, such as a pharmaceutically and physiologically acceptable fluid), under conditions that permit the antibody to specifically bind to the cancer cell surface protein. For example, the CTLA4-antibody-IR700 molecule, PD-L1-antibody-IR700 molecule, or combinations thereof, can be present in a pharmaceutically effective carrier, such as water, physiological saline, balanced salt solutions (such as PBS/EDTA), aqueous dextrose, sesame oil, glycerol, ethanol, combinations thereof, or the like, as a vehicle.

In some examples, the tumor-specific antibody-IR700 molecule and the CTLA4-antibody-IR700 molecule, PD-L1-antibody-IR700 molecule, or combinations thereof, are present in a single composition, and administered simultaneously. In other examples, the tumor-specific antibody-IR700 molecule and the CTLA4-antibody-IR700 molecule, PD-L1-antibody-IR700 molecule, or combinations thereof, are present in separate compositions, and administered simultaneously or contemporaneously, or sequentially (e.g., at least 1 minute, at least 5 minutes, at least 30 minutes, or at least 60 minutes in between).

In some examples, the subject is further administered a therapeutically effective amount of one or more immunoactivators, for example in combination with a therapeutically effective amount of one or more CTLA4 antibody-IR700 molecules, and/or one or more PD-L1 antibody-IR700 molecules. Immunoactivator include immune system activators and/or one or more inhibitors of immuno-suppressor cells (for example in the presence of a pharmaceutically acceptable carrier, such as a pharmaceutically and physiologically acceptable fluid). In a specific example, the Immunoactivator agent is IL-15. In another specific example, the immunomodulatory agent is interferon gamma. In some examples, the one or more immunoactivators are administered to the subject concurrently (for example, simultaneously or substantially simultaneously) with the one or more antibody-IR700 molecules, for example in the same composition. In other examples, the one or more antibody-IR700 molecules and the one or more immunoactivators are administered to the subject sequentially (in either order), for example, separated by at least about 1 hour of one another (for example, within about 5 minutes, about 10 minutes, about 15 minutes, about 20 minutes, about 30 minutes, about 40 minutes, about 50 minutes, about 60 minutes, about 2 hours, about 12 hours, about 24 hours, about 48 hours, about 3 days, about 4 days, about 5 days, about 6 days, or about 7 days).

In some examples, the subject is further administered a therapeutically effective amount of one or more reducing agents, for example to reduce edema that can result from NIR-PIT. In some examples, antibody-IR700 molecule(s) and the reducing agent(s) are present in a single composition, and administered simultaneously. In other examples, the antibody-IR700 molecule and reducing agent(s) are present in separate compositions, and administered simultaneously or contemporaneously, or sequentially (e.g., the tumor-specific antibody-IR700 molecule followed by the reducing agent(s), for example with at least 1 minute, at least 5 minutes, at least 30 minutes, at least 60 minutes, at least 12 hours, or at least 24 hours in between).

In some examples, the tumor-specific antibody-IR700 molecule is administered intravenously. In some examples, the CTLA4-antibody-IR700 molecule, PD-L1-antibody-IR700 molecule, or combinations thereof, are administered intravenously. In some examples, the one or more reducing agents is administered intraperitoneally. In some examples, the tumor-specific antibody-IR700 molecule and the CTLA4-antibody-IR700 molecule, PD-L1-antibody-IR700 molecule, or combinations thereof, are administered intravenously. In some examples, the tumor-specific antibody-IR700 molecule is administered intravenously and the one or more reducing agents is administered intraperitoneally. In some examples, the one or more immunoactivators (e.g., IL15) are administered intravenously. In one examples, the one or more immunoactivators is IL15 administered intravenously, such as at a dose of 1 to 10 mg. In some examples, the one or more immunoactivators (e.g., IFNγ) are administered intratumorally. In one examples, the one or more immunoactivators is IFNγ administered intratumorally, such as at a dose of no more than 1 mg, such as 0.01 to 1 mg.

After administering the one or more antibody-IR700 molecules (such as tumor specific antibody-IR700 molecules), the one or more antibody-IR700 molecules are allowed to accumulate in the targeted tumor or immune cells of the tumor. Similarly, after administering the one or more CTLA4-antibody-IR700 molecules, one or more PD-L1-antibody-IR700 molecules, or combinations thereof, such molecules are allowed to bind to their respective proteins (CTLA4 or PD-L1 respectively), for example to cells expressing such proteins, for example CTLA4- or PD-L1-expressing cells present in the tumor bed. The tumor cells and other cells in the tumor bed (or the subject having the cancer) are then irradiated under conditions that permit killing of the cells to which the antibody-IR700 conjugates are bound, for example irradiation at a wavelength of 660 to 740 nm (such as 600 to 710 nm) at a dose of at least 1 J/cm² (for example 680 nm or 690 nm at a dose of 10 to 60 J/cm², such as a dose of 20 to 50 J/cm² or 20 to 30 J/cm²).

In one example, there is at least about 10 minutes, at least about 30 minutes, at least about 1 hour, at least about 4 hours, at least about 8 hours, at least about 12 hours, at least about 24 hours, or at least about 48 hours (such as about 1 to 4 hours, 30 minutes to 1 hour, 10 minutes to 60 minutes, 30 minutes to 8 hours, 2 to 10 hours, 12 to 24 hours, 18 to 36 hours, or 24 to 48 hours) in between administering the antibody-IR700 conjugate(s) and the irradiation. In one example, the one or more antibody-IR700 conjugates are administered (e.g., i.v.) and at least about 10 minutes, at least about 30 minutes, at least about 1 hour, at least about 4 hours, at least about 8 hours, at least about 12 hours, at least about 24 hours, or at least about 48 hours (such as about 1 to 4 hours, 30 minutes to 1 hour, 10 minutes to 60 minutes, 30 minutes to 8 hours, 2 to 10 hours, 12 to 24 hours, 18 to 36 hours, or 24 to 48 hours, such as about 12 hours or about 24 hours) later, the tumor (or the subject) is irradiated. The one or more immunoactivators can be administered (for example systemically, such as iv or ip) before or after the one or more antibody-IR700 conjugates, and before or after the irradiation. In some examples, the one or more immunoactivators are administered before and after irradiation, for example, at least one dose of immunoactivators prior to irradiation and at least one dose of immunoactivators after irradiation (such as 24 hours before and one or more of 24, 48, 72, 96, or more hours after irradiation). In additional examples, a dose of immunoactivators may also be administered on the same day as at least one irradiation treatment. The one or more reducing agents can be administered (for example systemically, such as iv or ip) before or after the one or more antibody-IR700 conjugates but before the irradiation (such as at least 5 minutes before, at least 10 minutes before, at least 15 minutes before, or at least 30 minutes before, for example 5 to 60 minutes prior to irradiation).

In some examples, multiple doses of the one or more of the antibody-IR700 molecules are administered to the subject, such as at least 2, at least 3, at least 4, at least 5, at least 6, at least 7, at least 8, at least 9, or at least 10 separate doses (or administrations). In some examples, multiple doses of the one or more CTLA4 antibody-IR700 molecules, the one or more PD-L1 antibody-IR700 molecules, or combinations thereof, are administered to the subject, such as at least 2, at least 3, at least 4, at least 5, at least 6, at least 7, at least 8, at least 9, or at least 10 separate doses (or administrations). In some examples, multiple doses of the one or more immunoactivators are administered to the subject, such as at least 2, at least 3, at least 4, at least 5, at least 6, at least 7, at least 8, at least 9, or at least 10 separate doses (or administrations). In some examples, multiple doses of the one or more reducing agents are administered to the subject, such as at least 2, at least 3, at least 4, at least 5, at least 6, at least 7, at least 8, at least 9, or at least 10 separate doses (or administrations). In some examples, multiple doses of the irradiation with NIR are administered to the subject, such as at least 2, at least 3, at least 4, at least 5, at least 6, at least 7, at least 8, at least 9, or at least 10 separate doses (or administrations). In a specific example, the subject is administered at least one dose of the one or more of the antibody-IR700 molecule(s), at least two doses of one or more immunoactivators and at least two separate NIR irradiation administrations.

The NIR excitation light wavelength allows penetration of at least several centimeters into tissues. For example, by using fiber-coupled laser diodes with diffuser tips, NIR light can be delivered within several centimeters of otherwise inaccessible tumors located deep with respect to the body surface. In addition to treating solid cancers, circulating tumor cells (including, but not limited to hematological malignancies) can be targeted since they can be excited when they traverse superficial vessels (for example using the NIR LED wearable devices described herein).

In one example, the disclosed methods kill at least 10%, for example at least 20%, at least 40%, at least 50%, at least 80%, at least 90%, at least 95%, at least 98%, or more of the treated target cells (such as cancer cells expressing the tumor-specific antigen, or immune cells in the tumor bed, such as T cells expressing PD-L1 or CTLA4 in the tumor bed, such as 4CD4⁺Foxp3⁺ Tregs in the tumor bed expressing CTLA4), for example relative to the absence of treatment with the disclosed methods (e.g., without administration of one or more tumor-specific antibody-IR700 molecules in combination with (a) one or more CTLA4 antibody-IR700 molecules, one or more PD-L1 antibody-IR700 molecules, or combinations thereof, (b) one or more reducing agents, (c) one or more immunoactivators, or combinations of a, b, and c, and without NIR irradiation).

In one example, the disclosed methods selectively kill the cells that express the protein (such as a tumor-specific antigen, CTLA4, or PD-L1) that specifically binds to the antibody of the antibody-IR700 conjugate used, thereby treating the tumor. For example, by selective killing of tumor cells relative to normal cells, the methods are capable of killing tumor cells more effectively than normal cells such as, for example, cells not expressing the protein (such as a tumor-specific antigen) that specifically binds to the antibody administered. In some examples the disclosed methods decrease the weight, size and/or volume of a tumor, slow the growth of a tumor, decrease or slow recurrence of the tumor, decrease or slow metastasis of the tumor (for example by reducing the number of metastases or decreasing the weight/volume/size of a metastasis), or combinations thereof. For example, the disclosed methods in some examples reduce tumor size (such as weight or volume of a tumor) such as by at least 10%, at least 20%, at least 40%, at least 50%, at least 80%, at least 90%, at least 95%, at least 98%, or even 100%, for example relative to the absence of treatment with the disclosed methods (e.g., without administration of one or more antibody-IR700 molecules, in combination with (a) one or more reducing agents, (b) one or more immunoactivators, or combinations thereof, and/or without NIR irradiation). In some examples the disclosed methods decrease the weight, volume or size of a metastasis, such as by at least 10%, at least 20%, at least 40%, at least 50%, at least 80%, at least 90%, at least 95%, at least 98%, or even 100%, for example relative to the absence of treatment with the disclosed methods (e.g., without administration of one or more antibody-IR700 molecules, in combination with (a) one or more reducing agents, (b) one or more immunoactivators, or combinations thereof, and/or without NIR irradiation of the primary tumor). In some examples the disclosed methods have abscopal effects, and decrease the weight, volume or size of a metastasis that itself has not been irradiated at a wavelength of 660 to 740 nm at a dose of at least 1 J cm⁻² (such as at a dose of at least 4 J cm⁻² or at a dose of 4 to 50 J cm⁻²) and is located distant from the irradiated area of the tumor or lesion. In some examples the weight, volume or size of a non-irradiated metastasis decreases by at least 10%, at least 20%, at least 40%, at least 50%, at least 80%, at least 90%, at least 95%, at least 98%, or even 100% by treatment with the disclosed methods (e.g., without administration of one or more antibody-IR700 molecules, in combination with (a) one or more reducing agents, (b) one or more immunoactivators, or combinations thereof, and/or without NIR irradiation of the primary tumor).

By selective killing of Treg cells (such as CD4⁺Foxp3⁺ Tregs) relative to other immune cells, the methods are capable of killing Treg cells that are irradiated more effectively than other immune cells such as, for example, Tregs not in the tumor bed. In one example, the disclosed methods (such as those the use CTLA4 antibody-IR700) decreases Tregs (such as FOXP3⁺ CD4⁺ Treg cells in the tumor bed, but not in other parts of the body, such as the spleen or regional lymphnodes). For example, the disclosed methods (e.g., in example where CTLA4 antibody-IR700 or PD-L1 antibody-IR700 is administered) in some examples decrease the number of FOXP3⁺ CD4⁺ Treg cells in the tumor bed by at least 10%, for example by at least 20%, at least 40%, at least 50%, at least 80%, at least 90%, at least 95%, at least 98%, or more, relative to the absence of treatment with the disclosed methods (e.g., without administration of one or more antibody-IR700 molecules and/or without NIR irradiation). In some examples, the disclosed methods increase the ratio of CD4⁺Foxp3⁻ cells:CD4⁺Foxp3⁺ cells by at least 10%, for example by at least 20%, at least 40%, at least 50%, at least 80%, at least 90%, at least 200%, at least 300%, or more, relative to the absence of treatment the disclosed methods (e.g., without administration of one or more antibody-IR700 molecules and/or without NIR irradiation). In some examples, the disclosed methods do not substantially decrease the number of FOXP3⁺ CD4⁺ Treg cells in the spleen or regional lymph nodes of the tumor, for example decrease no more than 10%, no more than 5%, no more than 1%, or no more than 0.5%, relative to the absence of treatment with the disclosed methods (e.g., without administration of one or more antibody-IR700 molecules and/or without NIR irradiation).

In some examples, the disclosed methods decrease intra-tumoral blood perfusion by at least 10%, for example by at least 20%, at least 40%, at least 50%, at least 80%, at least 90%, at least 95%, at least 98%, or more, relative to the absence of treatment the disclosed methods (e.g., without administration of one or more antibody-IR700 molecules and/or without NIR irradiation).

In some examples, the disclosed methods decrease one or more symptoms associated with a tumor, a recurrence, and/or a metastatic tumor. In one example, the disclosed methods slow the growth of a tumor, such as by at least 10%, for example by at least 20%, at least 40%, at least 50%, at least 80%, at least 90%, or more, relative to the absence of treatment with the disclosed methods (e.g., without administration of one or more antibody-IR700 molecules and/or without NIR irradiation). In one example, the disclosed methods reduce or eliminates tumor recurrence, such as by at least 10%, for example by at least 20%, at least 40%, at least 50%, at least 80%, at least 90%, at least 95%, at least 98%, at least 99% or even 100%, relative to the absence of treatment with the disclosed methods (e.g., without administration of one or more antibody-IR700 molecules and/or without NIR irradiation).

In some examples, the disclosed methods increase a subject's (such as a subject with a tumor or who has had a tumor previously removed) survival time, for example relative to the absence of treatment with the disclosed methods (e.g., without administration of one or more antibody-IR700 molecules and/or without NIR irradiation). In some examples, the survival time of a subject increases at least 20%, at least 25%, at least 40%, at least 50%, at least 80%, at least 90%, or more, for example relative to the absence of treatment with the disclosed methods (e.g., without administration of one or more antibody-IR700 molecules and/or without NIR irradiation). In some examples, the survival time of a subject increases by at least 2 months, at least 3 months, at least 4 months, at least 5 months, at least 6 months, at least 9 months, at least 1 year, at least 1.5 years, at least 2 years, at least 3 years, at least 4 years, at least 5 years or more, for example relative to the absence of treatment with the disclosed methods (e.g., without administration of one or more antibody-IR700 molecules and/or without NIR irradiation). For example, the disclosed methods in some examples increase a subject's progression-free survival time or disease-free survival time (for example, lack of recurrence of the primary tumor or lack of metastasis) by at least 1 months, at least 2 months, at least 3 months, at least 6 months, at least 12 months, at least 18 months, at least 24 months, at least 36 months, at least 48 months, at least 60 months, or more, relative to average survival time in the absence of treatment with the disclosed methods (e.g., without administration of one or more antibody-IR700 molecules and/or without NIR irradiation).

In some examples, use of one or more reducing agents in combination with NIR-PIT reduces edema in the treated subject by at least 10%, at least 20%, at least 30%, at least 40%, at least 50%, at least 60%, at least 75%, at least 80%, at least 90%, or even at least 95%, as compared to the amount of edema without use of one or more reducing agents with NIR-PIT. In some examples, use of one or more reducing agents in combination with NIR-PIT reduces acute inflammatory reaction in the treated subject by at least 10%, at least 20%, at least 30%, at least 40%, at least 50%, at least 60%, at least 75%, at least 80%, at least 90%, or even at least 95%, as compared to the amount of acute inflammatory reaction without use of one or more reducing agents with NIR-PIT.

In one example, combinations of the effects listed above are achieved with the disclosed methods.

The disclosed methods can be used to treat fixed tumors in the body as well as hematological malignancies and/or tumors in the circulation (e.g., leukemia cells, metastases, and/or circulating tumor cells). However, circulating cells, by their nature, cannot be exposed to light for very long. Thus, if the cell to be killed is one that is circulating throughout the body, the methods can be accomplished by using a device that can be worn, or that covers parts of the body. For example, such a device can be worn for extended time periods. Everyday wearable items (e.g., wristwatches, jewelry (such as a necklace or bracelet), blankets, clothing (e.g., underwear, socks, and shoe inserts) and other everyday wearable items) which incorporate NIR emitting light emitting diodes (LEDs) and a battery pack, can be used. Such devices produce light on the skin underlying the device over long periods leading to continual exposure of light to superficial vessels over prolonged periods. Circulating tumor cells are exposed to the light as they transit thru the area underlying the device. As an example, a wristwatch or bracelet version of this device can include a series of NIR LEDs with battery power pack to be worn for most of the day. After administration of the one or more tumor-specific antibody-IR700 molecules (e.g., in combination with one or more CTLA4 antibody-IR700 molecules, one or more PD-L1 antibody-IR700 molecules, one or more reducing agents, one or more immunoactivators, or combinations thereof, systemically, such as iv and/or ip), circulating cells bind antibody-IR700 conjugates and become susceptible to killing by PIT. As these cells flow within the vessels adjacent to the LED present in the everyday wearable item (e.g., bracelet or wristwatch), they would be exposed to NIR light rendering them susceptible to cell killing. The dose of light may be adjustable according to diagnosis and cell type.

In some examples, the method further includes monitoring the therapy, such as killing of tumor cells, killing of Tregs, or both. In such examples, the subject is administered one or more antibody-IR700 molecules (such as tumor-specific antibody-IR700 molecules and/or immune cell specific antibody-IR700 molecules), for example in combination with one or more reducing agents, one or more immunoactivators, or combinations thereof, and irradiated as described herein. However, a lower dose of an antibody-IR700 conjugate and NIR light can be used for monitoring (as cell killing may not be required, just monitoring of the therapy). In one example, the amount of antibody-IR700 conjugate administered for monitoring is at least 2-fold less (such as at least 3-, 4-, 5-, 6-, 7-, 8-, 9-, or 10-fold less than the therapeutic dose). In one example, the amount of antibody-IR700 conjugate administered for monitoring is at least 20% or at least 25% less than the therapeutic dose. In one example, the amount of NIR light used for monitoring is at least 1/1000 or at least 1/10,000 of the therapeutic dose. This permits detection of the cells being treated. For example, by using such methods, the size of the tumor and metastases can be monitored.

In some examples, the method is useful during surgery, such as endoscopic procedures. For example, after the antibody-IR700 molecules (such as tumor-specific antibody-IR700 molecules and/or immune cell specific antibody-IR700 molecules) are administered, for example in combination with one or more reducing agents, one or more immunoactivators, or combinations thereof, and the cells/subject irradiated as described above, this not only results in cell killing, but permits a surgeon or other medical care provider to visualize the margins of a tumor, and help ensure that resection of the tumor (such as a tumor of the skin, breast, lung, colon, head and neck, or prostate) is complete and that the margins are clear. In some examples, a lower dose of the antibody-IR700 conjugate can be used for visualization, such as at least 2-fold less (such as at least 3-, 4-, 5-, 6-, 7-, 8-, 9-, or 10-fold less than the therapeutic dose).

The one or more antibody-IR700 molecules (such as tumor-specific antibody-IR700 molecules and/or immune cell specific antibody-IR700 molecules), one or more reducing agents, and the one or more immunoactivators, can be administered locally or systemically, for example to subjects having a tumor, such as a cancer, or who has had a tumor previously removed (for example via surgery). Although specific examples are provided, one skilled in the art will appreciate that alternative methods of administration of the disclosed therapeutic agents can be used. Such methods may include for example, the use of catheters or implantable pumps to provide continuous infusion over a period of several hours to several days into the subject in need of treatment.

In one example, the one or more antibody-IR700 molecules (such as tumor-specific antibody-IR700 molecules and/or immune cell specific antibody-IR700 molecules), one or more reducing agents, and the one or more immunoactivators are administered by parenteral means, including direct injection or infusion into a tumor (intratumorally) or organ (e.g., prostate). In some examples, the one or more antibody-IR700 molecules (such as tumor-specific antibody-IR700 molecules and/or immune cell specific antibody-IR700 molecules), one or more reducing agents, and the one or more immunoactivators are administered to the tumor by applying the agents to the tumor, for example by local injection of therapeutic agents, bathing the tumor in a solution containing the therapeutic agents, or by pouring the therapeutic agents onto the tumor.

In addition, or alternatively, the disclosed compositions can be administered systemically, for example intravenously, intramuscularly, subcutaneously, intradermally, intraperitoneally, subcutaneously, or orally, to a subject having a tumor (such as cancer). The one or more antibody-IR700 molecules (such as tumor-specific antibody-IR700 molecules and/or immune cell specific antibody-IR700 molecules), one or more reducing agents, and the one or more immunoactivators may be administered by the same or different routes. In one example, the one or more antibody-IR700 molecules are administered iv and the one or more reducing agents delivered intraperitoneally. In one example, the one or more antibody-IR700 molecules and the one or more immunoactivators are administered iv and/or intratumorally, and the one or more reducing agents delivered intraperitoneally. In another example, the one or more antibody-IR700 molecules (such as tumor-specific antibody-IR700 molecules and/or immune cell specific antibody-IR700 molecules), one or more reducing agents, and the one or more immunoactivators are administered systemically (for example, intravenously or intraperitoneally). In one example, the one or more antibody-IR700 molecules (such as tumor-specific antibody-IR700 molecules and/or immune cell specific antibody-IR700 molecules), one or more reducing agents, and the one or more immunoactivators are administered intraperitoneally. In one example, the one or more tumor-specific antibody-IR700 molecules, the one or more immunoactivators, and the one or more reducing agents are administered intravenously.

The dosages of the therapeutic agents provided herein to be administered to a subject are not subject to absolute limits, but may depend on the nature of the composition, its active ingredients and its potential unwanted side effects (e.g., immune response against the antibody), the subject being treated and the type of condition being treated, and the manner of administration. Generally the dose will be a therapeutically effective amount, such as an amount sufficient to achieve a desired biological effect, for example an amount that is effective to decrease the size (e.g., volume and/or weight) of the tumor, or attenuate further growth of the tumor, or decrease undesired symptoms of the tumor.

For intravenous administration of antibody-IR700 molecules (including tumor-specific antibody-IR700 molecules and CTLA4- and PD-L1-antibody-IR700 molecules), exemplary dosages for administration to a subject for a single treatment can range from 0.5 to 200 mg/60 kg of body weight, 1 to 100 mg/60 kg of body weight, 1 to 50 mg/60 kg of body weight, 1 to 20 mg/60 kg of body weight, for example about 1 or 2 mg/kg of body weight. In yet another example, a therapeutically effective amount of intraperitoneally or intratumorally administered antibody-IR700 molecules (including tumor-specific antibody-IR700 molecules and CTLA4- and PD-L1-antibody-IR700 molecules) is 10 μg to 5000 μg of antibody-IR700 molecule per 1 kg of body weight, such as 10 μg/kg to 1000 μg/kg, 10 μg/kg to 500 μg/kg, or 100 μg/kg to 1000 μg/kg. In one example, the dose of antibody-IR700 molecules (including tumor-specific antibody-IR700 molecules and CTLA4- and PD-L1-antibody-IR700 molecules) administered to a human patient is at least 50 mg, such as at least 100 mg, at least 300 mg, at least 500 mg, at least 750 mg, or even 1 g.

For intravenous administration of reducing agents, exemplary dosages for administration to a subject for a single treatment can range from 0.5 to 300 g/60 kg of body weight, 1 to 300 g/60 kg of body weight, 1 to 50 g/60 kg of body weight, 1 to 20 g/60 kg of body weight, 1 to 10 g/60 kg of body weight, 10 to 300 g/60 kg of body weight, such as 1, 2, 5, 10, 20, 50, 100, 200 or 300 g/60 kg of body weight. In yet another example, a therapeutically effective amount of intraperitoneally or intratumorally administered reducing agents is 10 mg to 5000 mg of reducing agents per 1 kg of body weight, such as 10 mg/kg to 1000 mg/kg, 10 mg/kg to 500 mg/kg, or 100 mg/kg to 1000 mg/kg. In one example, the dose of reducing agents administered to a human patient is at least 1 g, at least 10 g, at least 20 g, at least 50 g, at least 100 g, at least 200 g, at least 300 g, such as 1, 2, 5, 10, 20, 50, 100, 200 or 300 g.

For intravenous administration of immunoactivating agents, such as IL-15, exemplary dosages for administration to a subject for a single treatment can range from 0.1 to 100 mg, such as 1 to 10 mg or 0.5 to 20 mg. For intratumoral administration of immunoactivating agents, such as IFN gamma, exemplary dosages for administration to a subject for a single treatment can range from 0.01 to 1 mg, such as 0.1 to 0.5 mg or 0.05 to 5 mg.

Treatments with disclosed antibody-IR700 molecules (including tumor-specific antibody-IR700 molecules and CTLA4- and PD-L1-antibody-IR700 molecules), one or more reducing agents, and/or one or more immunoactivators can be completed in a single day, or may be done repeatedly on multiple days with the same or a different dosage. Repeated treatments may be done on the same day, on successive days, or every 1-3 days, every 3-7 days, every 1-2 weeks, every 2-4 weeks, every 1-2 months, or at even longer intervals. In some examples, the antibody-IR700 molecules, one or more reducing agents, and/or one or more immunoactivators are administered on the same day. In other examples, the antibody-IR700 molecules, one or more reducing agents, and/or one or more immunoactivators are administered on different days, such as the one or more immunoactivators administered the day before the antibody-IR700 molecules, or the antibody-IR700 molecules administered the day before the one or more reducing agents). In one non-limiting example, the one or more antibody-IR700 molecules and one or more immunoactivators are administered to the subject on the same day and repeated doses of the one or more immunoactivators (at the same or different dosing level) are administered to the subject (for example, 1, 2, 3, 4, 5, or more additional doses of the one or more immunoactivators) on successive days, or every 1-3 days, every 3-7 days, every 1-2 weeks, every 2-4 weeks, every 1-2 months, or at even longer intervals. In some examples, the amount of the repeated doses of the one or more immunoactivators is reduced compared to the initial dose (for example, reduced by 50%).

Exemplary treatment combinations are provided in Table 1.

TABLE 1
Exemplary treatment combinations

| | | Immuno- | Reducing |
|---|---|---|---|
| | Antibody-IR700 | activator(s) | agent(s) |
| Example 1 | tumor-specific antibody-IR700 | No | No |
| | and anti-CTLA4-IR700 | | |
| Example 2 | tumor-specific antibody-IR700 | No | No |
| | and anti-PD-L1-IR700 | | |
| Example 3 | EGFR antibody-IR700 and anti- | No | No |
| | CTLA4-IR700 | | |
| Example 4 | EGFR antibody-IR700 and anti- | No | No |
| | PD-L1-IR700 | | |
| Example 5 | panitumumab-IR700 and anti- | No | No |
| | CTLA4-IR700 | | |
| Example 6 | cetuximab-IR700 and anti- | No | No |
| | PD-L1-IR700 | | |
| Example 7 | tumor-specific antibody-IR700 | Yes | No |
| | and anti-CTLA4-IR700 | | |
| Example 8 | tumor-specific antibody-IR700 | Yes | No |
| | and anti-PD-L1-IR700 | | |
| Example 9 | EGFR antibody-IR700 and anti- | Yes | No |
| | CTLA4-IR700 | | |
| Example 10 | EGFR antibody-IR700 and anti- | Yes | No |
| | PD-L1-IR700 | | |
| Example 11 | panitumumab-IR700 and anti- | Yes | No |
| | CTLA4-IR700 | | |
| Example 12 | cetuximab-IR700 and anti- | Yes | No |
| | PD-L1-IR700 | | |
| Example 13 | tumor-specific antibody-IR700 | No | Yes |
| | and anti-CTLA4-IR700 | | |
| Example 14 | tumor-specific antibody-IR700 | No | Yes |
| | and anti-PD-L1-IR700 | | |
| Example 15 | EGFR antibody-IR700 and anti- | No | Yes |
| | CTLA4-IR700 | | |
| Example 16 | EGFR antibody-IR700 and anti- | No | Yes |
| | PD-L1-IR700 | | |
| Example 17 | panitumumab-IR700 and anti- | No | Yes |
| | CTLA4-IR700 | | |
| Example 18 | cetuximab-IR700 and anti- | No | Yes |
| | PD-L1-IR700 | | |
| Example 19 | tumor-specific antibody-IR700 | Yes | Yes |
| | and anti-CTLA4-IR700 | | |
| Example 20 | tumor-specific antibody-IR700 | Yes | Yes |
| | and anti-PD-L1-IR700 | | |
| Example 21 | EGFR antibody-IR700 and anti- | Yes | Yes |
| | CTLA4-IR700 | | |
| Example 22 | EGFR antibody-IR700 and anti- | Yes | Yes |
| | PD-L1-IR700 | | |
| Example 23 | panitumumab-IR700 and anti- | Yes | Yes |
| | CTLA4-IR700 | | |
| Example 24 | cetuximab-IR700 and anti- | Yes | Yes |
| | PD-L1-IR700 | | |
| Example 25 | Anti-PD-L1-IR700 | Yes | No |
| Example 26 | Anti-CTLA4-IR700 | Yes | No |
| Example 27 | ipilimumab-IR700 | Yes | No |
| Example 28 | tremelimumab-IR700 | Yes | No |
| Example 29 | atezolizumab-IR700 | Yes | No |
| Example 30 | avelumab-IR700 | Yes | No |
| Example 31 | Anti-PD-L1-IR700 | Yes | Yes |
| Example 32 | Anti-CTLA4-IR700 | Yes | Yes |
| Example 33 | ipilimumab-IR700 | Yes | Yes |
| Example 34 | tremelimumab-IR700 | Yes | Yes |
| Example 35 | atezolizumab-IR700 | Yes | Yes |
| Example 36 | avelumab-IR700 | Yes | Yes |
| Example 37 | tumor-specific antibody-IR700 | Yes | No |
| Example 38 | tumor-specific antibody-IR700 | Yes | Yes |
| Example 39 | tumor-specific antibody-IR700 | No | Yes |

In some examples, any one of Examples 7-13 and 19-38 the immunoactivator is IL-15. In some examples, any one of Examples 7-13 and 19-38 the immunoactivator is interferon gamma. In some examples, any one of Examples 14-24, 31-36 and 38-39 the reducing agent is L-sodium ascorbate.

In additional embodiments, the methods also include administering to the subject one or more additional therapeutic agents. As described in International Patent Application Publication No. WO 2013/009475 (incorporated by reference herein in its entirety), there is about an 8 hour window following irradiation (for example irradiation at a wavelength of 660 to 710 nm at a dose of at least 10 J/cm², at least 20 J/cm², at least 30 J/cm², at least 40 J/cm², at least 50 J/cm², at least 70 J/cm², at least 80 J/cm² or at least 100 J/cm², such as at least 10 to 100 J/cm²), during which uptake of additional agents (e.g., nano-sized agents, such as those about at least 1 nm in diameter, at least 10 nm in diameter, at least 100 nm in diameter, or at least 200 nm in diameter, such as 1 to 500 nm in diameter) by the PIT-treated cells is enhanced. Thus, one or more additional therapeutic agents can further be administered to the subject contemporaneously or sequentially with the PIT. In one example, the additional therapeutic agents are administered after the irradiation, for example, about 0 to 8 hours after irradiating the cell (such as at least 10 minutes, at least 30 minutes, at least 60 minutes, at least 2 hours, at least 3 hours, at least 4, hours, at least 5 hours, at least 6 hours, or at least 7 hours after the irradiation, for example no more than 10 hours, no more than 9 hours, or no more than 8 hours, such as 1 hour to 10 hours, 1 hour to 9 hours 1 hour to 8 hours, 2 hours to 8 hours, or 4 hours to 8 hours after irradiation). In another example, the additional therapeutic agents are administered just before the irradiation (such as about 10 minutes to 120 minutes before irradiation, such as 10 minutes to 60 minutes or 10 minutes to 30 minutes before irradiation). Additional therapeutic agents that can be used are discussed below.

In additional embodiments, methods are provided that permit detection or monitoring of cell killing in real-time. Such methods are useful for example, to ensure sufficient amounts of antibody-IR700 molecules (including tumor-specific antibody-IR700 molecules and CTLA4- and PD-L1-antibody-IR700 molecules), one or more reducing agents, and/or one or more immunoactivators, or sufficient amounts of irradiation, were delivered to the cell or tumor to promote cell killing. These methods permit detection of cell killing before morphological changes become evident. In one example, the methods include contacting a cell having a cell surface protein with a therapeutically effective amount of one or more antibody-IR700 molecules (including tumor-specific antibody-IR700 molecules and CTLA4- and PD-L1-antibody-IR700 molecules), one or more reducing agents, and/or one or more immunoactivators; irradiating the cell at a wavelength of 660 to 740 nm and at a dose of at least 10 J/cm²; and detecting the cell with fluorescence lifetime imaging about 0 to 48 hours after irradiating the cell (such as at least 1 hour, at least 2 hours, at least 4 hours, at least 6 hours, at least 12 hours, at least 18 hours, at least 24 hours, at least 36 hours, at least 48 hours, or at least 72 hours after irradiating the cell, for example 1 minute to 30 minutes, 10 minutes to 30 minutes, 10 minutes to 1 hour, 1 hour to 8 hours, 6 hours to 24 hours, or 6 hours to 48 hours after irradiating the cell), thereby detecting the cell killing in real-time. Shortening FLT serves as an indicator of acute membrane damage induced by PIT. Thus, the cell is irradiated under conditions sufficient to shorten IR700 FLT by at least 25%, such as at least 40%, at least 50%, at least 60% or at least 75%. In one example, the cell is irradiated at a wavelength of 660 nm to 740 nm (such as 680 nm to 700 nm, such as 680 or 690 nm) and at a dose of at least 10 J/cm², at least 20 J/cm², at least 30 J/cm², at least 40 J/cm², at least 50 J/cm² or at least 60 J/cm², such as 10 to 60 J/cm², 20 to 50 J/cm², 20 to 25 J/cm², or 30 to 50 J/cm².

In some examples, methods of detecting cell killing in real time includes contacting the cell with one or more additional therapeutic agents, for example about 0 to 8 hours after irradiating the cell. The real-time imaging can occur before or after contacting the cell with one or more additional therapeutic agents. For example, if insufficient cell killing occurs after administration of the one or more antibody-IR700 molecules (including tumor-specific antibody-IR700 molecules and CTLA4- and PD-L1-antibody-IR700 molecules), one or more reducing agents, and/or one or more immunoactivators, as determined by the real-timing imaging, then the cell can be contacted with one or more additional therapeutic agents. However, in some examples, the cell is contacted with the antibody-IR700 molecules (including tumor-specific antibody-IR700 molecules and CTLA4- and PD-L1-antibody-IR700 molecules), one or more reducing agents, and/or one or more immunoactivators, and additional therapeutic agents, prior to detecting the cell killing in real-time.

### Exemplary Cells

The target cell can be a cell that is not desired or whose growth is not desired, such as a cancer cell (e.g., a tumor cell) or an immune cell (e.g., T cell, such as a Treg). The cells can be present in a mammal to be treated, such as a subject (for example, a human or veterinary subject) with cancer. Any target cell can be treated with the claimed methods. In one example, the target cell expresses a cell surface protein that is not substantially found on the surface of other normal (desired) cells, an antibody can be selected that specifically binds to such protein, and an antibody-IR700 molecule generated for that protein. In one example, the cell surface protein is a tumor-specific protein (e.g., antigen), such as EGFR. In one non-limiting example, the cell surface protein is CTLA4 (for example to target CD4⁺Foxp3⁺ Tregs in the tumor bed). In one non-limiting example, the cell surface protein is PD-L1.

In one example, the tumor cell is a cancer cell, such as a cell in a patient with cancer. Exemplary cells that can be killed with the disclosed methods include cells of the following tumors: a hematological malignancy such as a leukemia, including acute leukemia (such as acute lymphocytic leukemia, acute myelocytic leukemia, and myeloblastic, promyelocytic, myelomonocytic, monocytic and erythroleukemia), chronic leukemias (such as chronic myelocytic (granulocytic) leukemia and chronic lymphocytic leukemia), polycythemia vera, lymphoma, Hodgkin's disease, non-Hodgkin's lymphoma, multiple myeloma, Waldenstrom's macroglobulinemia, heavy chain disease). In another example the cell is a solid tumor cell, such as cells from sarcomas and carcinomas, fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, pancreatic cancer, breast cancer, ovarian cancer, prostate cancer, hepatocellular carcinoma, lung cancer, colorectal cancer, squamous cell carcinoma, a head and neck cancer (such as head and neck squamous cell carcinoma), basal cell carcinoma, adenocarcinoma (for example adenocarcinoma of the pancreas, colon, ovary, lung, breast, stomach, prostate, cervix, or esophagus), sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS cancers (such as a glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma). In one example the tumor is an oropharyngeal cancer.

In a specific example, the cell is a lung cancer cell.

In a specific example, the cell is a breast cancer cell.

In a specific example, the cell is a colon cancer cell.

In a specific example, the cell is a head and neck cancer cell.

In a specific example, the cell is a prostate cancer cell.

In a specific example, the cell is an oropharyngeal cancer cell.

In a specific example, the cell is a squamous cell carcinoma cancer cell, such as one of a head and neck or skin cancer).

In some examples the cancer or cancer cell treated is one at a site distant from the tumor irradiated, such as a distant metastasis that does not receive irradiation at a wavelength of 660 to 740 nm and at a dose of at least 1 J/cm², such as one 1 mm away, 1 inch away, 2 inches away, 3 inches away, 4 inches away, 6 inches away, 12 inches away, or more, as the methods can have abscopal effects.

In some examples, the cancer or cancer cell treated is moderately or highly immunogenic, that is able to provoke an immune response. In some examples, a NIR-PIT method that includes administration of CTLA4 antibody-IR700 and/or PD-L antibody-IR700 is used to treat a moderately or highly immunogenic tumor. Examples of moderately or highly immunogenic cancers include melanoma, lung cancer (e.g., NSCLC), colon cancer, and renal cell cancer. In one example, a highly immunogenic cancer is a prostate cancer. In some examples, the cancer or cancer cell treated is not highly immunogenic, that is unable to provoke an immune response. In some examples, NIR-PIT with CTLA4 antibody-IR700 and/or PD-L antibody-IR700 is used in combination with anti-tumor-IL700 (such as anti-EGFR-IR700 or anti-PSA antigen-IR700) to treat a low immunogenic tumor. Examples of low immunogenic cancers include oral squamous cell cancer, breast cancer, pancreatic cancer, and multiple myeloma. In some examples, a tumor immunogenicity score is used to determine whether a cancer is low, moderately, or highly immunogenic (e.g., see Wang et al, eLife, 8:e49020, 2019).

### Exemplary Subjects

In some examples the disclosed methods are used to treat a subject who has cancer or a subject with a tumor, such as a tumor described herein. In some examples, the tumor has been previously treated, such as surgically or chemically removed, and the disclosed methods are used subsequently to kill any remaining undesired tumor cells that may remain in the patient and/or reduce recurrence or metastasis of the tumor.

The disclosed methods can be used to treat any mammalian subject (such as a human or veterinary subject, such as a dog or cat), such as a human, who has a tumor, such as a cancer, or has had such previously removed or treated. Subjects in need of the disclosed therapies can include human subjects having cancer, such as a cancer that express a tumor-specific protein on the cell surface that can specifically bind to the tumor-specific antibody-IR700 molecule, or a cancer infiltrated with immune cells that can bind to an immune cell specific antibody-IR700 molecule (e.g., CTLA4 antibody-IR700). For example, the disclosed methods can be used as initial treatment for cancer either alone, or in combination with radiation or other chemotherapy or surgery. The disclosed methods can also be used in patients who have failed previous radiation or chemotherapy. Thus, in some examples, the subject is one who has received other therapies, but those other therapies have not provided a desired therapeutic response. The disclosed methods can also be used in patients with localized and/or metastatic cancer and/or a recurrence of a primary tumor.

In some examples the method includes selecting a subject that will benefit from the disclosed therapies, such as selecting a subject having a tumor that expresses a cell surface protein (such as a tumor-specific protein) that can specifically bind to an antibody-IR700 molecule. For example, if the subject is determined to have a breast cancer that expresses HER2, the subject can be selected to be treated with an anti-HER2-IR700 molecule, such as Trastuzumab-IR700, for example in combination with CTLA4 antibody-IR700 molecules, PD-L1 antibody-IR700 molecules, one or more reducing agents, one or more immunoactivators, or combinations thereof, and the subject is subsequently irradiated as described herein.

### Exemplary Cell Surface Proteins

In one example, the protein on the cell surface of the target cell to be killed is not present in significant amounts on other cells. For example, the cell surface protein can be a receptor that is only found on the target cell type.

In a specific example, the cell surface protein is a cancer- or tumor-specific protein (also referred to as a tumor-specific antigen or tumor-associated antigen), such as members of the EGF receptor family (e.g., HER1, 2, 3, and 4) and cytokine receptors (e.g., CD20, CD25, IL-13R, CD5, CD52, etc.). Thus, in some examples, the cell surface protein is an antigen expressed on the cell membrane of tumor cells. Tumor-specific proteins are proteins that are unique to cancer cells or are much more abundant on them, as compared to other cells, such as normal cells. For example HER2 is primarily found in breast cancers, while HER1 is primarily found in adenocarcinomas, which can be found in many organs, such as the pancreas, breast, prostate and colon.

Exemplary tumor-specific proteins that can be found on a target cell (and to which an antibody specific for that protein can be used to formulate an antibody-IR700 molecule), include but are not limited to: any of the various MAGEs (Melanoma-Associated Antigen E), including MAGE 1 (e.g., GenBank Accession Nos. M77481 and AAA03229), MAGE 2 (e.g., GenBank Accession Nos. L18920 and AAA17729), MAGE 3 (e.g., GenBank Accession Nos. U03735 and AAA17446), MAGE 4 (e.g., GenBank Accession Nos. D32075 and A06841.1), etc.; any of the various tyrosinases (e.g., GenBank Accession Nos. U01873 and AAB60319); mutant ras; mutant p53 (e.g., GenBank Accession Nos. X54156, CAA38095 and AA494311); p97 melanoma antigen (e.g., GenBank Accession Nos. M12154 and AAA59992); human milk fat globule (HMFG) associated with breast tumors (e.g., GenBank Accession Nos. S56151 and AAB19771); any of the various BAGEs (Human B melanoma-Associated Antigen E), including BAGE1 (e.g., GenBank Accession No. Q13072) and BAGE2 (e.g., GenBank Accession Nos. NM_182482 and NP_872288), any of the various GAGEs (G antigen), including GAGE1 (e.g., GenBank Accession No. Q13065) or any of GAGE2-6; various gangliosides, CD25 (e.g., GenBank Accession Nos. NP_000408.1 and NM_000417.2).

Other tumor-specific antigens include the HPV 16/18 and E6/E7 antigens associated with cervical cancers (e.g., GenBank Accession Nos. NC_001526, FJ952142.1, ADB94605, ADB94606, and U89349), mucin (MUC 1)-KLH antigen associated with breast carcinoma (e.g., GenBank Accession Nos. J03651 and AAA35756), CEA (carcinoembryonic antigen) associated with colorectal cancer (e.g., GenBank Accession Nos. X98311 and CAA66955), gp100 (e.g., GenBank Accession Nos. S73003 and AAC60634) associated with for example melanoma, MART1 antigens associated with melanoma (e.g., GenBank Accession No. NP_005502), cancer antigen 125 (CA125, also known as mucin 16 or MUC16) associated with ovarian and other cancers (e.g., GenBank Accession Nos. NM_024690 and NP_078966); alpha-fetoprotein (AFP) associated with liver cancer (e.g., GenBank Accession Nos. NM_001134 and NP_001125); Lewis Y antigen associated with colorectal, biliary, breast, small-cell lung, and other cancers; tumor-associated glycoprotein 72 (TAG72) associated with adenocarcinomas; glypican 1 (GPC1 associated with pancreatic cancer, glioma, and breast cancer), glypican 2 (associated with neuroblastoma) and glypican 3 (associated with hepatocellular carcinoma), and the PSA antigen associated with prostate cancer (e.g., GenBank Accession Nos. X14810 and CAA32915).

Other exemplary tumor-specific proteins include, but are not limited to, PMSA (prostate membrane specific antigen; e.g., GenBank Accession Nos. AAA60209 and AAB81971.1) associated with solid tumor neovasculature, as well prostate cancer; HER-2 (human epidermal growth factor receptor 2, e.g., GenBank Accession Nos. M16789.1, M16790.1, M16791.1, M16792.1 and AAA58637) associated with breast cancer, ovarian cancer, stomach cancer and uterine cancer, HER-1 (e.g., GenBank Accession Nos. NM_005228 and NP_005219) associated with lung cancer, anal cancer, and gliobastoma as well as adenocarcinomas; NY-ESO-1 (e.g. GenBank Accession Nos. U87459 and AAB49693) associated with melanoma, sarcomas, testicular carcinomas, and other cancers, hTERT (aka telomerase) (e.g., GenBank Accession. Nos. NM_198253 and NP_937983 (variant 1), NM_198255 and NP_937986 (variant 2)); proteinase 3 (e.g., GenBank Accession Nos. M29142, M75154, M96839, X55668, NM 00277, M96628, X56606, CAA39943 and AAA36342), and Wilms tumor 1 (WT-1, e.g. GenBank Accession Nos. NM_000378 and NP_000369 (variant A), NM_024424 and NP_077742 (variant B), NM_024425 and NP_077743 (variant C), and NM_024426 and NP_077744 (variant D)).

In one example the tumor-specific protein is EGFR, and in some examples the EGFR antibody-IR700 is or includes panitumumab or cetuximab.

In one example the tumor-specific protein is PD-L1, and in some examples the PD-L1 antibody-IR700 is or includes atezolizumab, avelumab, durvalumab, or cosibelimab.

In one example the tumor-specific protein is CD52 (e.g., GenBank Accession. Nos. AAH27495.1 and CAI15846.1) associated with chronic lymphocytic leukemia; CD33 (e.g., GenBank Accession. Nos. NM_023068 and CAD36509.1) associated with acute myelogenous leukemia; and CD20 (e.g., GenBank Accession. Nos. NP_068769 NP_031667) associated with Non-Hodgkin lymphoma.

In a specific example, the tumor-specific protein is CD44 (e.g., OMIM 107269, GenBank Accession. Nos. ACI46596.1 and NP_000601.3). CD44 is a marker of cancer stem-like cells and various types of cancers and is implicated in intercellular adhesion, cell migration, cell spatial orientation, and promotion of matrix-derived survival signal. High expression of CD44 on the plasma membrane of tumors can be associated with tumor aggressiveness and poor outcome.

Thus, the disclosed methods can be used to treat any cancer that expresses a tumor-specific protein.

### Exemplary Antibody-IR700 Molecules

Because cell surface protein sequences are publically available (for example as described above), making or purchasing antibodies (or other small molecules that can be conjugated to IR700) specific for such proteins can be accomplished. For example, if the tumor-specific protein HER2 is selected as a target, antibodies specific for HER2 (such as Trastuzumab) can be purchased or generated and attached to the IR700 dye. Other specific examples are provided in Table 2 and elsewhere herein. In one example, the antibody conjugated to IR700 is a humanized monoclonal antibody.

In one example, the antibody-IR700 molecule is a PD-L1-antibody-IR700 molecule, such as atezolizumab-IR700, avelumab-IR700, durvalumab-IR700, cosibelimab-IR700, KN035-IR700, BMS935559-IR700, MEDI-4736-IR700, MEDfI-4737-IR700, BMS-936559-IR700, MPDL-3280A-IR700, or CK-301-IR700.

In one example the antibody-IR700 molecule is a EGFR-antibody-IR700 molecule, such as, panitumumab-IR700 or cetuximab-IR700.

Antibody-IR700 molecules can be generated using methods such as those described in WO 2013/009475 (incorporated by reference herein in its entirety).

TABLE 2
Exemplary tumor-specific antigens and antibodies

| Tumor-Specific | | |
|---|---|---|
| Antigen | Exemplary Tumors | Exemplary Antibody/Small Molecules |
| HER1 | Adenocarcinoma (e.g., | Cetuximab, panitumumab, zalutumumab, |
| | colorectal cancer, head and | nimotuzumab, matuzumab, necitumumab, |
| | neck cancer) | imgatuzumab, 806. Small molecule inhibitors |
| | | gefitinib, erlotinib, and lapatinib can also be |
| | | conjugated to IR700. |
| HER2 | breast cancer, ovarian | Trastuzumab (Herceptin ®), pertuzumab |
| | cancer, stomach cancer, | (Perjeta ®, Omnitarg ®) |
| | uterine cancer | |
| HER3 | Breast, colon, lung, | Patritumab, Duligotumab, MM-121 |
| | ovarian, prostate, and head | |
| | and neck squamous cell | |
| | cancer | |
| CA 242 | Colorectal, gastric, | Mouse clone M62862 and M62861 |
| | pancreatic, hepatocellular | |
| | cancer | |
| CD19 | B cell lymphoma, CLL, | GBR 401, MEDI-551, Blinatumomab |
| | ALL | (Blincyto ®) |
| CD20 | Non-Hodgkin lymphoma | Tositumomab (Bexxar ®); Rituximab |
| | | (Rituxan, Mabthera); Ibritumomab |
| | | tiuxetan (Zevalin, for example in |
| | | combination with yttrium-90 or |
| | | indium-111 therapy); Ofatumumab |
| | | (Arzerra ®), veltuzumab, obinutuzumab, |
| | | ublituximab, ocaratuzumab |
| CD22 | Non-Hodgkin's | Narnatumab, inotuzumab ozogamicin, |
| | lymphoma, CLL, hairy cell | moxetumomab pasudotox |
| | leukemia, ALL, solid | |
| | tumors | |
| CD25 | T-cell lymphoma | Daclizumab (Zenapax), Basiliximab |
| CD30 | Hodgkin's lymphoma | Brentuximab vedotin (ADCETRIS ®), |
| | | iratumumab |
| CD33 | Acute myelogenous | Gemtuzumab (Mylotarg, for example in |
| | leukemia | combination with calicheamicin therapy); |
| | | lintuzumab |
| CD37 | CLL, non-Hodgkin | Otlertuzumab |
| | lymphoma, mantle cell | |
| | lymphoma | |
| CD38 | Multiple myeloma | Daratumumab |
| CD40 | Multiple myeloma, non- | Lucatumumab, dacetuzumab |
| | Hodgkin's or Hodgkin's | |
| | lymphoma | |
| CD44 | Cancer stem cells, breast, | bivatuzumab |
| | prostate, renal, head and | RG3756 |
| | neck cancer, lymphoma, | |
| | leukemia | |
| CD52 | chronic lymphocytic | Alemtuzumab (Campath) |
| | leukemia | |
| CD56 | Small cell lung cancer, | Lorvotuzumab mertansine |
| | ovarian cancer | |
| CD70 | Renal cell carcinoma | Vorsetuzumab mafodotin |
| CD74 | Multiple myeloma | Milatuzumab |
| CD140 | Glioblastoma, non-small | Tovetumab |
| | cell lung cancer | |
| CAIX | Renal cell carcinoma | Girentuximab, cG250 |
| CEA | colorectal cancer, some | Arcitumomab (CEA-scan (Fab fragment, |
| | gastric cancers, biliary | approved by FDA), colo101; Labetuzumab |
| | cancer | (CEA-Cide ®) |
| Cancer antigen 125 | ovarian cancer, | OC125 monoclonal antibody |
| (CA125) | mesothelioma, breast | |
| | cancer | |
| Alpha-fetoprotein | hepatocellular carcinoma | ⁹⁰Y-tacatuzumab tetraxetan; ab75705 |
| (AFP) | | (available from Abcam) and other |
| | | commercially available AFP antibodies |
| Cytokeratin | Colorectal cancer | ^{99m}Tc- Votumumab (HUMASPECT ®) |
| EGFL7 | Non-small cell lung cancer, | Parsatuzumab |
| | colorectal cancer | |
| EpCAM | Epithelial tumors (breast, | IGN101, oportuzumab monatox, tucotuzumab |
| | colon and lung) | celmoleukin, adecatumumab |
| EPHA3 | Lung, kidney and colon | KB004, IIIA4 |
| | tumors, melanoma, glioma | |
| | and hematological | |
| | malignancies | |
| FAP | Colon, breast, lung, | Sibrotuzumab, F19 |
| | pancreas, and head and | |
| | neck tumors | |
| Fibronectin | Hodgkin's lymphoma | Radretumab |
| Folate-binding | Ovarian cancer | MOv18 and MORAb-003 (farletuzumab) |
| protein | | |
| Folate receptor | Ovarian cancer | Farletuzumab |
| alpha | | |
| Frizzled receptor | Breast, pancreatic, non- | Vantictumab |
| | small cell lung cancer | |
| Gangliosides | Neuroectodermal tumors | 3F8, ch14.18, KW-2871 |
| (e.g., GD2, GD3 | and some epithelial tumors | |
| and GM2) | | |
| gpA33 | Colorectal cancer | huA33 |
| glypican-1 | pancreatic cancer, glioma, | A-10, clone 01a033 |
| | and breast cancer) | |
| glypican-2 | neuroblastoma | CT3, hCT3 |
| glypican-3 | HCC | YP7, hYP7, GC33 |
| HGF | Solid tumors | Rilotumumab, ficlatuzumab |
| IGF1R | Glioma, lung, breast, head | Cixutumumab, dalotuzumab, figitumumab, |
| | and neck, prostate and | ganitumab, robatumumab, teprotumumab, |
| | thyroid cancer | AVE1642, IMC-A12, MK-0646, R1507, and |
| | | CP 751871 |
| IGLF2 | Breast cancer; | Dusigitumab |
| | Hepatocellular carcinoma; | |
| | Solid tumors | |
| IL-6 | renal cell cancer, prostate | Siltuximab |
| | cancer, Castleman's | |
| | disease | |
| Integrin αVβ3 | Tumor vasculature | Etaracizumab (ABEGRIN ®), intetumumab |
| Integrin α5β1 | Tumor vasculature | Volociximab |
| Lewis Y | colorectal cancer, biliary | B3 (Humanized), hu3S193, IgN311 |
| | cancer | |
| Mesothelin | Mesothelioma, pancreatic | Amatuximab |
| | cancer | |
| MET | Breast, ovarian, and lung | AMG 102, METMAB, SCH 900105 |
| | cancer | |
| Mucins | Breast, colon, lung and | Pemtumomab (THERAGYN ®), cantuzumab |
| | ovarian cancer | mertansine, ⁹⁰Y clivatuzumab tetraxetan, |
| | | oregovomab (OVAREX ®) |
| PDGFR-alpha | Soft tissue sarcoma | Olaratumab |
| Phosphatidylserine | Breast, pancreatic, | Bavituximab |
| | prostate, non-small cell | |
| | lung cancer, hepatocellular | |
| | carcinoma | |
| PSMA | Prostate cancer | J591 |
| RANKL | Prostate cancer, bone | Denosumab (XGEVA ®) |
| | metastases | |
| Scatter factor | Non-small cell lung, | Onartuzumab |
| receptor kinase | stomach, glioblastoma | |
| SLAMF7 (CD319) | Multiple myeloma | Elotuzumab |
| Syndecan 1 | Multiple myeloma, breast, | Indatuximab ravtansine |
| | bladder cancer | |
| TAG72 | adenocarcinomas including | B72.3 (FDA-approved monoclonal antibody), |
| | colorectal, pancreatic, | CC49 (minretumomab) |
| | gastric, ovarian, | |
| | endometrial, mammary, | |
| | and non-small cell lung | |
| | cancer | |
| Tenascin | Glioma, breast and prostate | 81C6 |
| | tumors | |
| TRAILR1 | Colon, lung and pancreas | Mapatumumab (HGS-ETR1) |
| | tumors and hematological | |
| | malignancies | |
| TRAILR2 | Non-small cell lung cancer, | Conatumumab, lexatumumab, |
| | non-Hodgkin's lymphoma, | mapatumumab, tigatuzumab, HGS-ETR2, |
| | multiple myeloma | CS-1008 |
| Vascular | Colorectal cancer | Bevacizumab (Avastin ®) |
| endothelial growth | | |
| factor | | |
| VEGFR | Epithelium-derived solid | IM-2C6, CDP791 |
| | tumors | |
| VEGFR2 | Gastric, non-small cell | Ramucirumab (Cyramza ™) |
| | lung, colorectal cancer | |
| Vimentin | Brain cancer | Pritumumab |

Additional antibodies that can be conjugated to IR700, and used in the disclosed methods, include 3F8, Abagovomab, Afutuzumab, Alacizumab, Altumomab pentetate, Anatumomab mafenatox, Apolizumab, Bectumomab, Belimumab, Besilesomab, Capromab pendetide, Catumaxomab, Citatuzumab bogatox, Detumomab, Ecromeximab, Eculizumab, Edrecolomab, Epratuzumab, Ertumaxomab, Galiximab, Glembatumumab vedotin, Igovomab, Imciromab, Lumiliximab, Mepolizumab, Metelimumab, Mitumomab, Morolimumab, Nacolomab tafenatox, Naptumomab estafenatox, Nofetumomab merpentan, Pintumomab, Satumomab pendetide, Sonepcizumab, Taplitumomab paptox, Tenatumomab, TGN1412, Ticilimumab (tremelimumab), TNX-650, or Tremelimumab.

In some examples, the cell surface protein recognized by the antibody conjugated to IR700, is one found on an immune cell, such as a Treg. In one example the protein is CTLA4. In one example, the antibody-IR700 molecule is a CTLA4-antibody-IR700 molecule, such as ipilimumab-IR700 or tremelimumab-IR700.

In one example, a patient is treated with at least two different antibody-IR700 molecules specific for cancer cell surface antigens. In one example, the two different antibody-IR700 molecules are specific for the same protein (such as HER-2), but are specific for different epitopes of the protein (such as epitope 1 and epitope 2 of HER-2). In another example, the two different antibody-IR700 molecules are specific for two different proteins or antigens. For example, anti-HER1-IR700 and anti-HER2-IR700 could be injected together as a cocktail to facilitate killing of cells bearing either HER1 or HER2.

### Immunomodulators/Immunoactivators

Immunoactivators of use in the disclosed methods include agents or compositions that activate the immune system and/or inhibit immuno-suppressor cells (also referred to herein as suppressor cells). Inhibition of immuno-suppressor cells and/or activation of immune responses may increase tumor cell killing and leads to production of memory T cells, which can provide a “vaccine” effect against recurrences and/or distant tumors or metastases. In some examples, one or more immunoactivators are used in combination with one or more tumor-specific antibody-IR700 molecules, one or more reducing agents, or both.

In some embodiments, the immunoactivator is an inhibitor of immuno-suppressor cells, for example, an agent that inhibits or reduces activity of immuno-suppressor cells. In some cases, the immunomodulator kills immuno-suppressor cells. In some examples, the immuno-suppressor cells are regulatory T (Treg) cells. In some examples, not all of the suppressor cells are killed in vivo, as such could lead to development of autoimmunity. Thus, in some examples, the method reduces the activity or number of immuno-suppressor cells in an area of subject, such as in the area of a tumor or an area that used to have a tumor, by at least 50%, at least 60%, at least 75%, at least 80%, at least 90%, or at least 95%. In some examples, the method reduces the total number of suppressor cells in a subject by at least 50%, at least 60%, at least 75%, at least 80%, at least 90%, or at least 95%.

Inhibitors of immuno-suppressor cells include tyrosine kinase inhibitors (such as sorafenib, sunitinib, and imatinib), chemotherapeutic agents (such as cyclophosphamide or interleukin-toxin fusions, for example denileukin difitox (IL2-diphtheria toxin fusion), or anti-CD25 antibodies (such as daclizumab or basiliximab) or other antibodies that bind to suppressor cell surface proteins (such as those described below). In other examples, inhibitors of immuno-suppressor cells include immune checkpoint inhibitors, for example, anti-PD-L1 antagonizing antibodies, thereby preventing PD-L1 from binding to PD-1 (referred to herein as PD-1/PD-L1 mAb-mediated immune checkpoint blockade (ICB)). Thus, in some examples, the immunomodulator is a PD-L1 antagonizing antibody, such as atezolizumab, avelumab, durvalumab, cosibelimab, KN035 (envafolimab), BMS-936559, BMS935559, MEDI-4736, MPDL-3280A, or MEDI-4737 (or other example provided herein). Checkpoint inhibitors also include CTLA-4 antibodies, including ipilimumab and tremelimumab. The inhibitor of immuno-suppressor cells can also be a LAG-3 or B7-H3 antagonist, such as BMS-986016, and MGA271. In some examples, two or more of the inhibitors of immuno-suppressor cells can be administered to a subject. In one non-limiting example, a subject is administered an anti-PD1 and an anti-LAG-3 antibody.

In some examples, the agent that inhibits or reduces activity of suppressor cells includes one or more antibody-IR700 molecules, wherein the antibody specifically binds to a suppressor cell surface protein (such as CD25, CD4, C-X-C chemokine receptor type 4 (CXCR4), C-C chemokine receptor type 4 (CCR4), cytotoxic T-lymphocyte-associated protein 4 (CTLA4), glucocorticoid induced TNF receptor (GITR), OX40, folate receptor 4 (FR4), CD16, CD56, CD8, CD122, CD23, CD163, CD206, CD11b, Gr-1, CD14, interleukin 4 receptor alpha chain (IL-4Ra), interleukin-1 receptor alpha (IL-1Ra), interleukin-1 decoy receptor, CD103, fibroblast activation protein (FAP), CXCR2, CD33, and CD66b)).

In a non-limiting example, the immunomodulator is a CD25 antibody-IR700 molecule, such as daclizumab-IR700 or basiliximab-IR700.

In other embodiments, the immunomodulator is an immune system activator (immunoactivator). In some examples, an immune system activator stimulates (activates) one or more T cells and/or natural killer (NK) cells. In one example, the immune system activator includes one or more interleukins (IL), such as IL-2, IL-15, IL-7, IL-12, and/or IL-21. In a non-limiting example, the immunomodulator includes or consists of IL-15. In a non-limiting example, the immunomodulator includes IL-2 and IL-15. In a non-limiting example, the immunomodulator includes or consists of interferon gamma. In another example, the immune system activator includes one or more agonists to co-stimulatory receptors, such as 4-1BB, OX40, or GITR. In a non-limiting example, the immunomodulator includes stimulatory anti-4-1BB, anti-OX40, and/or anti-GITR antibodies.

In some examples, one or more (such as 1, 2, 3, 4, 5, or more) doses of the immunomodulator is administered to the subject. Thus, administering the immunomodulator can be completed in a single day, or may be done repeatedly on multiple days with the same or a different dosage (such as administering at least 2 different times, 3 different times, 4 different times 5 different times or 10 different times). In some examples, the repeated administrations are the same dose. In other examples, the repeated administrations are different does (such as a subsequent dose that is higher than the preceding dose or a subsequent dose that is lower than the preceding dose). Repeated administration of the immunomodulator may be done on the same day, on successive days, every other day, every 1-3 days, every 3-7 days, every 1-2 weeks, every 2-4 weeks, every 1-2 months, or at even longer intervals. In some examples, at least one dose of the immunomodulator is administered prior to irradiation and at least one dose of the immunomodulator is administered after irradiation.

### Irradiation

After the subject is administered one or more antibody-IR700 molecules, the subject (or a tumor in the subject) is irradiated. As only cells expressing the target protein will be recognized by the antibody, only those cells will have sufficient amounts of the antibody-IR700 molecules associated with it to it to kill the cells. This decreases the likelihood of undesired side effects, such as killing of non-tumor cells, as the irradiation will only kill the cells to which the antibody-IR700 molecules are bound, not the other cells.

The subject (for example, a tumor in the subject) is irradiated with a therapeutic dose of radiation at a wavelength of 660-740 nm, such as 660-710 nm, 660-700 nm, 680-700 nm, 670-690 nm, for example, 680 nm or 690 nm. In particular examples, the cells, the tumor, or the subject is irradiated at a dose of at least 1 J/cm², such as at least 10 J/cm², at least 20 J/cm², at least 25 J/cm², at least 30 J/cm², at least 50 J/cm², at least 100 J/cm², or at least 500 J/cm², for example, 1-1000 J/cm², 1-500 J/cm², 1-100 J/cm², 4-50 J/cm², 30-50 J/cm², 10-100 J/cm², 20-30 J/cm², 20-50 J/cm² or 10-50 J/cm².

The subject can be irradiated one or more times. Thus, irradiation can be completed in a single day, or may be done repeatedly on multiple days with the same or a different dosage (such as irradiation at least 2 different times, 3 different times, 4 different times 5 different times or 10 different times). In some examples, the repeated irradiations are the same dose. In other examples, the repeated irradiations are different does (such as a subsequent dose that is higher than the preceding dose or a subsequent dose that is lower than the preceding dose). Repeated irradiations may be done on the same day, on successive days, every other day, every 1-3 days, every 3-7 days, every 1-2 weeks, every 2-4 weeks, every 1-2 months, or at even longer intervals. In one example, a first irradiation is 50 J/cm² and a second irradiation is at 100 J/cm², where the irradiations are on consecutive days (for example, about 24 hours apart). In one example, a first irradiation is 10-50 J/cm² and a second irradiation is at 10-50 J/cm², where the irradiations are on consecutive days (for example, about 24 hours apart).

In some examples, the irradiation is provided with a wearable device incorporating an NIR LED. In other examples, another type of device that can be used with the disclosed methods is a flashlight-like device with NIR LEDs. Such a device can be used for focal therapy of lesions during surgery, or incorporated into endoscopes to apply NIR light to body surfaces after the administration of one or more PIT agents. Such devices can be used by physicians or qualified health personnel to direct treatment to particular targets on the body.

### Treatment Using Wearable NIR LEDs

As described herein, the disclosed methods are highly specific for cancer cells (or Tregs in the tumor bed, for example when CTLA4-antibody-IR700 is used). However, to kill the cells circulating in the body or present on the skin, the patient can wear a device that incorporates an NIR LED. In some examples, the patient uses at least two devices, for example an article of clothing or jewelry during the day, and a blanket at night. In some example the patient uses at least two devices at the same time, for example two articles of clothing. These devices make it possible to expose the patient to NIR light using portable everyday articles of clothing and jewelry so that treatment remains private and does not interfere with everyday activities. In some examples, the device can be worn discreetly during the day for PIT therapy. Exemplary devices incorporating an NIR LED are disclosed in International Patent Application Publication No. WO 2013/009475 (incorporated by reference herein).

In one example, the patient is administered one or more antibody-IR700 molecules, for example in combination with one or more reducing agents and/or one or more immunoactivators using the methods described herein. The patient then wears a device that incorporates an NIR LED, permitting long-term therapy and treatment of tumor cells that are present in the blood or lymph or on the skin. In some examples, the dose is at least at least 1 J/cm², at least at least 4 J/cm², at least 10 J/cm², at least 20 J/cm², at least 30 J/cm², at least 40 J/cm², or at least 50 J/cm², such as 10 to 100 J/cm², 10 to 50 J/cm², such as 20 J/cm² or 30 J/cm². In some examples, administration of the one or more antibody-IR700 molecules, for example in combination with one or more reducing agents and/or one or more immunoactivators, is repeated over a period of time (such as bi-weekly or monthly), to ensure therapeutic levels are present in the body.

In some examples, the patient wears or uses the device, or combination of devices, for at least 1 week, such as at least 2 weeks, at least 4 weeks, at least 8 weeks, at least 12 weeks, at least 4 months, at least 6 months, or even at least 1 year. In some examples, the patient wears or uses the device, or combination of devices, for at least 4 hours a day, such as at least 12 hours a day, at least 16 hours a day, at least 18 hours a day, or 24 hours a day. It is quite possible that multiple devices of a similar “everyday” nature (blankets, bracelets, necklaces, underwear, socks, shoe inserts) could be worn by the same patient during the treatment period. At night the patient can use the NIR LED blanket or other covering.

### Administration of Additional Therapies

As discussed above, prior to, during, or following administration of one or more antibody-IR700 molecules, for example in combination with one or more reducing agents and/or one or more immunoactivators, and irradiation, the subject can receive one or more other therapies. In one example, the subject receives one or more treatments to remove or reduce the tumor prior to administration of the one or more antibody-IR700 molecules, for example in combination with one or more reducing agents and/or one or more immunoactivators, and irradiation. In other examples, additional treatments or therapeutic agents (such as anti-neoplastic agents) can be administered to the subject to be treated, for example, after the irradiation, for example, about 0 to 8 hours after irradiating the cell (such as at least 10 minutes, at least 30 minutes, at least 60 minutes, at least 2 hours, at least 3 hours, at least 4, hours, at least 5 hours, at least 6 hours, or at least 7 hours after the irradiation, for example no more than 10 hours, no more than 9 hours, or no more than 8 hours, such as 1 hour to 10 hours, 1 hour to 9 hours 1 hour to 8 hours, 2 hours to 8 hours, or 4 hours to 8 hours after irradiation). In another example, the additional therapeutic agents are administered just before the irradiation (such as about 10 minutes to 120 minutes before irradiation, such as 10 minutes to 60 minutes or 10 minutes to 30 minutes before irradiation).

Examples of such therapies that can be used in combination with the disclosed methods, which in some examples, enhance accessibility of the tumor to additional therapeutic agents for about 8 hours after the PIT, include but are not limited to, surgical treatment for removal or reduction of the tumor (such as surgical resection, cryotherapy, or chemoembolization), as well as anti-tumor pharmaceutical treatments which can include radiotherapeutic agents, anti-neoplastic chemotherapeutic agents, antibiotics, alkylating agents and antioxidants, kinase inhibitors, and other agents. In some examples, the additional therapeutic agent is conjugated to a nanoparticle. Particular examples of additional therapeutic agents that can be used include microtubule binding agents, DNA intercalators or cross-linkers, DNA synthesis inhibitors, DNA and/or RNA transcription inhibitors, antibodies, enzymes, enzyme inhibitors, and gene regulators. These agents (which are administered at a therapeutically effective amount) and treatments can be used alone or in combination.

“Microtubule binding agent” refers to an agent that interacts with tubulin to stabilize or destabilize microtubule formation thereby inhibiting cell division. Examples of microtubule binding agents that can be used in conjunction with the disclosed methods include, without limitation, paclitaxel, docetaxel, vinblastine, vindesine, vinorelbine (navelbine), the epothilones, colchicine, dolastatin 15, nocodazole, podophyllotoxin and rhizoxin. Analogs and derivatives of such compounds also can be used. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

The following classes of compounds can be used with the methods disclosed herein: suitable DNA and/or RNA transcription regulators, including, without limitation, actinomycin D, daunorubicin, doxorubicin and derivatives and analogs thereof also are suitable for use in combination with the disclosed therapies. DNA intercalators and cross-linking agents that can be administered to a subject include, without limitation, cisplatin, carboplatin, oxaliplatin, mitomycins, such as mitomycin C, bleomycin, chlorambucil, cyclophosphamide and derivatives and analogs thereof. DNA synthesis inhibitors suitable for use as therapeutic agents include, without limitation, methotrexate, 5-fluoro-5′-deoxyuridine, 5-fluorouracil and analogs thereof. Examples of suitable enzyme inhibitors include, without limitation, camptothecin, etoposide, formestane, trichostatin and derivatives and analogs thereof. Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone and derivatives and analogs thereof. Kinase inhibitors include Gleevec® (imatinib), Iressa® (gefitinib), and Tarceva® (erlotinib) that prevent phosphorylation and activation of growth factors.

Non-limiting examples of anti-angiogenic agents include molecules, such as proteins, enzymes, polysaccharides, oligonucleotides, DNA, RNA, and recombinant vectors, and small molecules that function to reduce or even inhibit blood vessel growth. Examples of suitable angiogenesis inhibitors include, without limitation, angiostatin KI-3, staurosporine, genistein, fumagillin, medroxyprogesterone, suramin, interferon-alpha, metalloproteinase inhibitors, platelet factor 4, somatostatin, thromobospondin, endostatin, thalidomide, and derivatives and analogs thereof. For example, in some embodiments the anti-angiogenesis agent is an antibody that specifically binds to VEGF (e.g., Avastin, Roche) or a VEGF receptor (e.g., a VEGFR2 antibody). In one example the anti-angiogenic agent includes a VEGFR2 antibody, or DMXAA (also known as Vadimezan or ASA404; available commercially, e.g., from Sigma Corp., St. Louis, MO) or both. The anti-angiogenic agent can be bevacizumab, sunitinib, an anti-angiogenic tyrosine kinase inhibitors (TKI), such as sunitinib, xitinib and dasatinib. These can be used individually or in any combination.

Other therapeutic agents, for example anti-tumor agents, that may or may not fall under one or more of the classifications above, also are suitable for administration in combination with the disclosed methods. By way of example, such agents include adriamycin, apigenin, rapamycin, zebularine, cimetidine, and derivatives and analogs thereof.

In some examples, the subject receiving the therapeutic antibody-IR700 molecule is also administered interleukin-2 (IL-2), for example via intravenous administration. In particular examples, IL-2 (Chiron Corp., Emeryville, CA) is administered at a dose of at least 500,000 IU/kg as an intravenous bolus over a 15 minute period every eight hours beginning on the day after administration of the antibody-IR700 molecules and continuing for up to 5 days. Doses can be skipped depending on subject tolerance.

Exemplary additional therapeutic agents include anti-neoplastic agents, such as chemotherapeutic and anti-angiogenic agents or therapies, such as radiation therapy. In one example the agent is a chemotherapy immunosuppressant (such as Rituximab, steroids) or a cytokine (such as GM-CSF). Exemplary chemotherapeutic agents are provided in, for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Combination chemotherapy is the administration of more than one agent to treat cancer.

Exemplary chemotherapeutic agents that can be used with the methods provided herein include but are not limited to, carboplatin, cisplatin, paclitaxel, docetaxel, doxorubicin, epirubicin, topotecan, irinotecan, gemcitabine, iazofurine, gemcitabine, etoposide, vinorelbine, tamoxifen, valspodar, cyclophosphamide, methotrexate, fluorouracil, mitoxantrone, Doxil (liposome encapsulated doxiorubicine) and vinorelbine. Additional examples of chemotherapeutic agents that can be used include alkylating agents, antimetabolites, natural products, or hormones and their antagonists. Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). Specific non-limiting examples of alkylating agents are temozolomide and dacarbazine. Examples of antimetabolites include folic acid analogs (such as methotrexate), pyrimidine analogs (such as 5-FU or cytarabine), and purine analogs, such as mercaptopurine or thioguanine. Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide). Examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone).

Exemplary chemotherapy drugs include Adriamycin, Alkeran, Ara-C, BiCNU, Busulfan, CCNU, Carboplatinum, Cisplatinum, Cytoxan, Daunorubicin, DTIC, 5-fluoruracil (5-FU), Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, Gemcitabine (Gemzar), Herceptin, Irinotecan (Camptosar, CPT-11), Leustatin, Navelbine, Rituxan STI-571, Taxotere, Topotecan (Hycamtin), Xeloda (Capecitabine), Zevelin and calcitriol. Non-limiting examples of immunoactivators that can be used include AS-101 (Wyeth-Ayerst Labs.), bropirimine (Upjohn), gamma interferon (Genentech), GM-CSF (granulocyte macrophage colony stimulating factor; Genetics Institute), IL-2 (Cetus or Hoffman-LaRoche), human immune globulin (Cutter Biological), IMREG (from Imreg of New Orleans, La.), SK&F 106528, and TNF (tumor necrosis factor; Genentech).

In some examples, the additional therapeutic agent is conjugated to (or otherwise associated with) a nanoparticle, such as one at least 1 nm in diameter (for example at least 10 nm in diameter, at least 30 nm in diameter, at least 100 nm in diameter, at least 200 nm in diameter, at least 300 nm in diameter, at least 500 nm in diameter, or at least 750 nm in diameter, such as 1 nm to 500 nm, 1 nm to 300 nm, 1 nm to 100 nm, 10 nm to 500 nm, or 10 nm to 300 nm in diameter).

In one example, at least a portion of the tumor (such as a metastatic tumor) is surgically removed (for example via surgical resection and/or cryotherapy), irradiated (for example administration of radioactive material or energy (such as external beam therapy) to the tumor site to help eradicate the tumor or shrink it), chemically treated (for example via chemoembolization) or combinations thereof, prior to administration of the disclosed therapies (such as administration of one or more tumor-specific antibody-IR700 molecules, for example in combination with one or more reducing agents and/or one or more immunoactivators, and irradiation). For example, a subject having a metastatic tumor can have all or part of the tumor surgically excised prior to administration of the disclosed therapies. In an example, one or more chemotherapeutic agents are administered following treatment with one or more antibody-IR700 molecules, for example in combination with one or more reducing agents and/or one or more immunoactivators, and irradiation, and irradiation. In another particular example, the subject has a metastatic tumor and is administered radiation therapy, chemoembolization therapy, or both concurrently with the administration of the disclosed therapies.

In some examples, the additional therapeutic agent administered is a monoclonal antibody, for example, 3F8, Abagovomab, Adecatumumab, Afutuzumab, Alacizumab, Alemtuzumab, Altumomab pentetate, Anatumomab mafenatox, Apolizumab, Arcitumomab, Bavituximab, Bectumomab, Belimumab, Besilesomab, Bevacizumab, Bivatuzumab mertansine, Blinatumomab, Brentuximab vedotin, Cantuzumab mertansine, Capromab pendetide, Catumaxomab, CC49, Cetuximab, Citatuzumab bogatox, Cixutumumab, Clivatuzumab tetraxetan, Conatumumab, Dacetuzumab, Detumomab, Ecromeximab, Eculizumab, Edrecolomab, Epratuzumab, Ertumaxomab, Etaracizumab, Farletuzumab, Figitumumab, Galiximab, Gemtuzumab ozogamicin, Girentuximab, Glembatumumab vedotin, Ibritumomab tiuxetan, Igovomab, Imciromab, Intetumumab, Inotuzumab ozogamicin, Ipilimumab, Iratumumab, Labetuzumab, Lexatumumab, Lintuzumab, Lorvotuzumab mertansine, Lucatumumab, Lumiliximab, Mapatumumab, Matuzumab, Mepolizumab, Metelimumab, Milatuzumab, Mitumomab, Morolimumab, Nacolomab tafenatox, Naptumomab estafenatox, Necitumumab, Nimotuzumab, Nofetumomab merpentan, Ofatumumab, Olaratumab, Oportuzumab monatox, Oregovomab, Panitumumab, Pemtumomab, Pertuzumab, Pintumomab, Pritumumab, Ramucirumab, Rilotumumab, Rituximab, Robatumumab, Satumomab pendetide, Sibrotuzumab, Sonepcizumab, Tacatuzumab tetraxetan, Taplitumomab paptox, Tenatumomab, TGN1412, Ticilimumab (tremelimumab), Tigatuzumab, TNX-650, Trastuzumab, Tremelimumab, Tucotuzumab celmoleukin, Veltuzumab, Volociximab, Votumumab, Zalutumumab, or combinations thereof.

### Exemplary Reducing Agents

Exemplary reducing agents that can be used in the methods provided herein include agents that loose (or “donate”) an electron to an electron recipient (oxidizing agent) in a redox chemical reaction. Exemplary reducing agents that can be used in the methods provided herein include L-cysteine, L-sodium ascorbate (L-NaAA), ascorbic acid (such as L- or R-ascorbic acid) and glutathione. In some examples, the reducing agent used in the disclosed methods is not sodium azide. In some examples, the reducing agent used in the disclosed methods is not L-cysteine. In one example, the reducing agent is L-NaAA (such as 5 to 50 g i.p.).

### Example 1

### Materials and Methods

This example describes materials and methods used to obtain the results in Example 2.

### Cell Culture

Parental mEERL cells were established by transduction of HPV 16 E6/E7 and hRAS to C57BL/6-derived oropharyngeal epithelial cells (Hoover et al., Arch OtolaryngolHead Neck Surg 2007; 133: 495-502; Spanos et al., J Virol 2008; 82:2493-500; Williams et al., Head Neck 2009; 31:911-8). mEERL-hEGFR were from Dr. William C. Spanos (Sanford Research; Okada et al., EBioMedicine 2021; 67:103345). In addition, MC38 cells (colon cancer, RRID: CVCL_B288; from Claudia Palena, N C I, 2015) and MOC2 cells (oral cancer, RRID: CVCL_ZD33; from Kerafast) stably expressing luciferase (MC38-luc and MOC2-luc, generated via stable transduction with RediFect Red-Fluc lentivirus from PerkinElmer per the manufacturer's recommendations), and LL/2-luc cells (Lewis lung carcinoma, RRID: CVCL_A4CM; purchased from Imanis Life Sciences) were used. High luciferase expression on the MC38-luc, MOC2-luc, and LL/2-luc cells were confirmed through 10 passages. mEERL-hEGFR cells were cultured in DMEM/F-12 (Thermo Fisher Scientific) supplemented with 10% FBS, 1% penicillin-streptomycin (Thermo Fisher Scientific), and 1× human keratinocyte growth supplement (Thermo Fisher Scientific), which was modified from a previous report (Mermod et al., Int J Cancer 2018; 142:2518-28). MC38-luc and LL/2-luc cells were cultured in RPMI 1640 supplemented with 10% FBS and 1% penicillin-streptomycin. MOC2-luc cells were cultured in the mixture of IMDM medium and Ham's Nutrient Mixture F12 Media (at a ratio of 2:1, GE Health Healthcare Life Sciences) supplemented with 5% fetal bovine serum, 1% penicillin-streptomycin, 5 ng/mL insulin (MilliporeSigma), 40 ng/mL hydrocortisone (MilliporeSigma), and 3.5 ng/mL human recombinant EGF (MilliporeSigma). All cells were cultured in a humidified incubator at 37° C. in an atmosphere of 95% air and 5% CO₂ cultured for no more than 30 passages. Cell line identity was tested via short tandem repeat (STR) profiling. For MC38-luc, MOC2-luc, and LL2-luc, the matching score was above 80% indicating the cell line identity was authentic. For mEERL-hEGFR, STR profile did not match any known cell lines. Mycoplasma testing was performed by PCR for MC38-luc, MOC2-luc and LL2-luc, by MycoAlert PLUS Mycoplasma Detection Kit for mEERL-hEGFR and all the cell lines were tested negative.

### Reagents

Water-soluble, silicon phthalocyanine derivative, IRDye700DX NHS ester (IR700), was from LI-COR Bioscience (Lincoln, NE). Panitumumab, a fully humanized IgG2 mAb directed against hEGFR, was from Amgen (Thousand Oaks, CA). Anti-mouse/human CD44 (clone IM7; RRID: AB_110649) anti-mouse CTLA-4 (cytotoxic T lymphocyte antigen-4) (clone 9D9; RRID: AB_10949609) were from Bio X Cell (Lebanon, NH). All other chemicals were of reagent grade.

### Synthesis of IR700-Conjugated Panitumumab, Anti-CD44, and Anti-CTLA-4

Conjugation of IR700 with monoclonal antibodies (mAbs) was performed according to previous reports. Briefly, 1 mg of either mAb was incubated with 5-fold molar excess of IR700 NIH ester (10 mmol/L in DMSO) 0.1 mol/L Na₂HPO₄ (pH 8.5) at room temperature for 1 hour. The mixture was purified with a filtration column (Sephadex G 25 column, PD-10; GE Healthcare). The quality of APC was evaluated with sodium dodecyl sulfate polyacrylamide gel electrophoresis (SDS-PAGE) with a 4% to 20% gradient polyacrylamide gel (Life Technologies). Nonconjugated antibody was used for the control. After electrophoresis at 80 V for 2.5 hours, the gel was observed with a Pearl Imager (LI-COR Biosciences) using the 700 nm fluorescence channel. The gel was then stained with colloidal blue to compare the molecular weight of the conjugate to that of nonconjugated antibody. IR700-conjugated panitumumab, anti-CD44 antibody, and anti-CTLA4 antibody are abbreviated herein as pan-IR700, CD44-IR700, and CTLA4 IR700, respectively.

### Animal and Tumor Model

All procedures were performed in compliance with the Guide for the Care and Use of Laboratory Animals and approved by the local Animal Care and Use Committee. 6- to 8-week-old female C57BL/6 mice (IMSR_JAX: 000664)) were from The Jackson Laboratory. During invasive procedures, mice were anesthetized with inhaled 3% isoflurane and/or via intraperitoneal injection of 750 mg sodium pentobarbital (Nembutal Sodium Solution, Ovation Pharmaceuticals Inc.).

The lower part of the body of the mice was shaved before NIR light irradiation and image analysis. Tumors were established via subcutaneous injection of 1×10⁶ cells in the right or both side of dorsal flank for each model. Mice with tumors reaching approximately 50-100 mm³ in volume were used for the experiments. Mice were monitored each day, and tumor volume (tumor volume=length×width²×0.5) was measured twice a week until the tumor volume reached 2,000 mm³, whereupon the mice were euthanized with inhalation of carbon dioxide gas. In a bilateral model, the mice were euthanized when either tumor reached its humane endpoint. Tumor disappearance for 4 weeks or longer after treatment was defined as complete remission.

### In Vitro NIR-PIT

mEERL-hEGFR, MC38-luc, and LL/2-luc cells (2×10⁵) were seeded into 12-well plates, incubated for 24 hours, and then exposed to media containing pan-IR700 or CD44-IR700 (10 μg/mL) for 1 hours at 37° C. After washing with PBS, phenol-red-free RPMI 1640 medium was added. NIR light (690 nm) was irradiated to cancer cells with an ML7710 laser system (Modulight, Tampere, Finland) at a power density of 150 mW/cm². One hour after NIR-PIT, the cells were collected with trypsin, and stained with propidium iodide (PI, 1 μg/mL) at room temperature for 5 minutes, and then assessed for PI positivity on a BD FACSCalibur (BD Biosciences) using CellQuest software (BD Biosciences) using FlowJo software (FlowJo LLC). To assess cell viability, cell proliferation was evaluated by 3-(4,5-Dimethyl-2-thiazolyl)-2,5-diphenyl-2H-tetrazolium bromide (MTT) assay. Cells were incubated and treated, as described above. One hour after NIR-PIT, the medium was removed and 500 μl of MTT reagent (SIGMA Aldrich; 0.5 mg/ml) was added to each well. After 1 hour incubation, the supernatant was removed and 500 μL of 2-propanol was added to each well to dissolve the crystal formazan dye. After transferring 100 μL of the supernatant to 96 well plate each, absorbance was measured at 570 nm on a microplate reader (Synergy™ H1). For relative quantification, the value of absorbance in each group was normalized to that in the control group.

### In Vivo NIR-PIT

For the dual-targeted NIR-PIT experiment, tumor-bearing mice were randomized into four groups and intravenous injection of mixed antibody/APC followed by NIR light exposure was performed as follows: (i) mixed 25 mg panitumumab and 25 mg anti-CTLA4-antibody (I.V. group); (ii) mixed 25 mg pan-IR700 and 25 mg anti-CTLA4-antibody (panitumumab NIR-PIT group); (iii) mixed 25 mg panitumumab and 25 mg CTLA4-IR700 (CTLA4 NIR-PIT group); and (iv) mixed 25 mg pan-IR700 and 25 mg CTLA4-IR700 (dual NIR-PIT group). For the monotherapy of CTLA4-targeted NIR-PIT experiment, tumor-bearing mice were also randomized into three groups as follows: (i) no treatment (control group); (ii) intravenous administration of CTLA4-IR700 (25 mg) without NIR light exposure (APC I.V. group); and (iii) intravenous administration of CTLA4-IR700 (25 mg) followed by NIR light exposure (NIR-PIT group). The mixed antibody/APC was injected 6 days after inoculation of cancer cells into C57BL/6 mice. Twenty-four hours after administration, NIR light (690 nm, 150 mW/cm²) was irradiated to tumors at 50 J/cm² in all groups. The surface of the mouse other than the tumor was covered with aluminum foil. The mice cleared tumors in dual NIR-PIT group were re-challenged via subcutaneous injection of hEGFRmEERL (1×10⁶) cells in the contralateral flank. In the bilateral model, NIR light (690 nm, 150 mW/cm²) was given only to right-sided tumors at 50 J/cm² and the remainder of the mouse (including the left-sided tumor) was covered with aluminum foil during irradiation.

### Histologic Analysis

Tumors from mEERL-hEGFR, MC38-luc, and LLC tumors were harvested, formalin-fixed and paraffin-embedded, and sectioned at 4 mm. Following standard hematoxylin and eosin (HE) staining, bright-light photomicrographs were obtained using Mantra Quantitative Pathology Workstation (PerkinElmer).

### Multiplex Immunohistochemistry

Multiplex immunohistochemistry (IHC) was performed using Opal 7-Color AutomationIHC Kit (AKOYA Bioscience) and BOND RXm auto stainer (Leica Biosystems). The following antibodies were used: anti-CD8 (clone EPR20305; Abcam, 1:500 dilution), anti-CD4 (clone EPR19514; Abcam, 1:1,000 dilution), anti-FoxP3 (clone 1054C; Novus Biologicals, 1:1,000 dilution), anti-pan cytokeratin (rabbit poly; Bioss, 1:500 dilution). The staining was performed according to the Opal 7 color protocol provided by manufacturer with following modification: (i) antigen retrieval was performed using BOND ER2 solution (Leica Biosystems) for 20 minutes and (ii) the ImmPRESS HRP anti-Rabbit IgG (Peroxidase) Polymer Detection Kit (Vector Laboratories) was used instead of anti-mouse/human secondary antibody provided in the kit. Stained slides were mounted with VECTASHIELD Hardset Antifade Mounting Medium (Vector Laboratories) and then imaged using Mantra Quantitative Pathology Workstation (PerkinElmer). Images were analyzed with inForm software (AKOYA Biosystems). InForm software was trained to automatically detect tissues and cell phenotype according to following criteria: areas with pan-cytokeratin expression=tumor, other areas=stroma, CD4+FoxP3+ cells=Tregs, CD4+FoxP3−=CD4 T cells, or CD8+=CD8+ T cells, respectively. Cell count of each phenotype was exported and shown as count per megapixel area. Three tumor samples were tested for each group. Five pictures were taken for each specimen and cell count and tissue area were combined for all five pictures.

### Flow Cytometric Analysis

To evaluate expression of CTLA4, the mEERL-hEGFR tumor bearing-mice were euthanized when established tumor volume reached approximately 150 mm³. To confirm the loss of CTLA4 expressing cells after NIR-PIT, the tumors or spleen of mEERL-hEGFR tumor bearing-mice were harvested 3 hours after NIR light exposure. Tumor draining lymph nodes and spleens were also analyzed to evaluate for systemic effects. To assess the immune reaction in the regional lymph nodes, an ipsilateral inguinal lymph node, was harvested 2 days after NIR-PIT. Single-cell suspensions from tumor samples were prepared using the following protocol. Whole tumors were incubated in the RPMI 1640 medium (Thermo Fisher Scientific) containing collagenase type IV (1 mg/mL; Thermo Fisher Scientific) and DNase I (20 mg/mL; Millipore Sigma) at 37° C. for 30 minutes, then gently cut with scissors and mashed with the back of the plunger of 3 mL syringe. The tissues were passed through 70 μm cell strainer (Corning, Corning, NY, USA). Splenocytes were also analyzed to determine the systemic effect. A total of 3.0×10⁶ cells was stained and data for 5.0×10⁵ cells were collected for each tumor. The cells were stained with antibodies from either BioLegend [anti-CD3e (145-2C11, RRID:AB_312660), anti-CD8α(53-6.7, RRID: AB_2888883), anti-CD11b (M1/70, RRID: AB_312791), anti-CD11c (N418, RRID: AB_314173), anti-CD25 (PC61.5, RRID: AB_312847), anti-F4/80 (BM8, RRID: AB_893481), anti-CD45 (30-F11, RRID: AB_2563598), and anti-IA/I-E (M5/114.15.2, RRID: AB_313328)] or from Thermo Fisher Scientific [anti-CD4 (RM4-5, RRID: AB_464902), anti-CD69 (H1.2F3, RRID: AB_1210795), and anti-NK1.1 (136, RRID: AB_2534431)]. To distinguish live from dead cells, cells were also stained with LIVE/DEAD Fixable Dead Cell Stain (Thermo Fisher Scientific). To assess CTLA4 expression, anti-CTLA4 (9D9) or an isotype control [murine IgG2b (MPC-11, RRID: AB_1107791); Bio X Cell] was conjugated with Alexa Flour 647 NHS ester (Thermo Fisher Scientific). The conjugation was performed with the same method as IR700 conjugation. For staining FoxP3, the cells were fixed and permeabilized with FoxP3 Transcription Factor Staining Buffer Set (Thermo Fisher Scientific) followed by incubation with anti-FoxP3 (FJK-16s; Thermo Fisher Scientific). The fluorescence of the cells was then analyzed with the flow cytometer (FACSCalibur or FACSLyric, RRID: SCR_000401, BD Biosciences) and FlowJo software (FlowJo LLC, RRID: SCR_008520). Dead cells were removed from analysis based on FSC, SSC, and staining with LIVE/DEAD Fixable Dead Cell Stain. The Treg population was defined by gating for CD4bFoxP3b T cells among CD3b T cells.

### Statistical Analysis

Quantitative data were expressed as means±SEM. For in vitro experiments, a one-way analysis of variance (ANOVA) followed by the Tukey test was used. For in vivo experiments, a two-way ANOVA followed by the Tukey test was used for multiple comparisons (more than 3 groups). The cumulative probability of survival was analyzed by the Kaplan-Meier survival curve analysis, and the results were compared with the log-rank test. Statistical analysis was performed with GraphPad Prism version 8 (GraphPad software). A P value of less than 0.05 was considered statistically significant.

### Example 2

### Simultaneously Combined Cancer Cell- and CTLA4-Targeted NIR-PIT Causes a Synergistic Treatment Effect in Syngeneic Mouse Models

This example describes results using methods that reduce Treg-mediated immunosuppression by Treg-cell killing using CTLA4-targeted NIR-PIT in combination with tumor-targeted NIR-PIT (e.g., using a tumor-specific antibody-IR700 conjugate, such as anti-EGFR-IR700 conjugate). Combined NIR-PIT targeting cytotoxic T-lymphocyte antigen 4 (CTLA4) expressing cells and cancer cells was investigated in four tumor models including a newly established hEGFR-expressing murine oropharyngeal cancer cell (mEERL-hEGFR). While single molecule-targeted therapy (NIR-PIT targeting hEGFR or CTLA4) did not inhibit tumor progression in poorly immunogenic mEERLhEGFR tumor, dual (CTLA4/hEGFR)-targeted NIR-PIT significantly suppressed tumor growth and prolonged survival resulting in a 38% complete response rate. After the dual-targeted NIR-PIT, depletion of CTLA4 expressing cells, which were mainly regulatory T cells (Tregs), and an increase in the CD8b/Treg ratio in the tumor bed were observed, indicating enhanced host antitumor immunity. Furthermore, dual-targeted NIR-PIT showed antitumor immunity in distant untreated tumors of the same type. Thus, simultaneous cancer cell-targeted NIR-PIT and CTLA4-targeted NIR-PIT is a new cancer therapy strategy, especially in poorly immunogenic tumors where NIR-PIT monotherapy is suboptimal.

Efficacy of Panitumumab and CTLA-4 NIR-PIT for mEERL-hEGFR Cells In Vitro

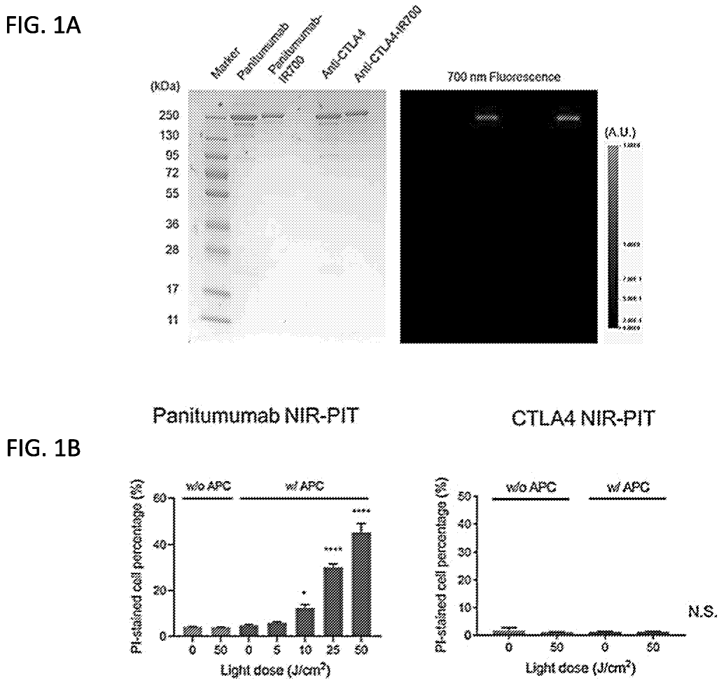

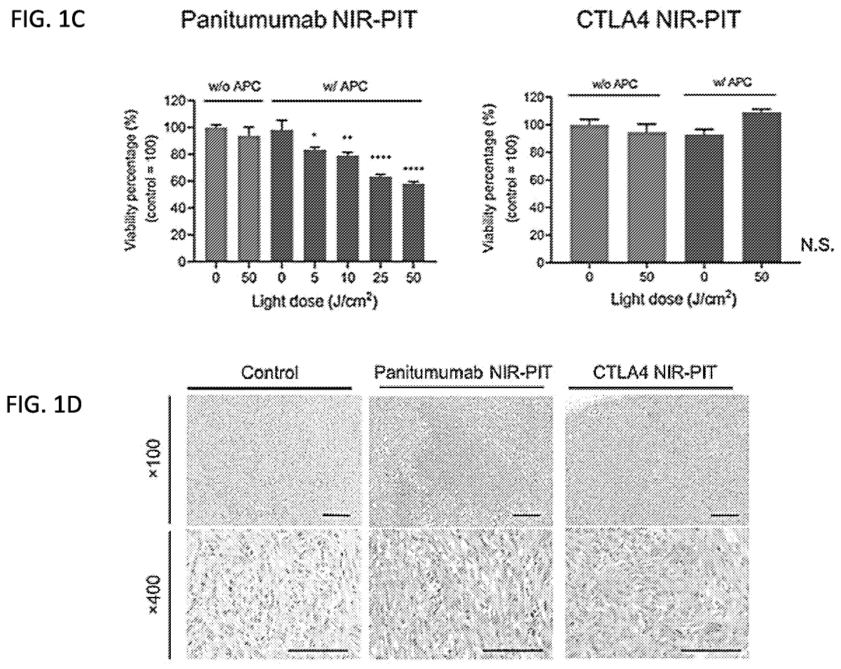

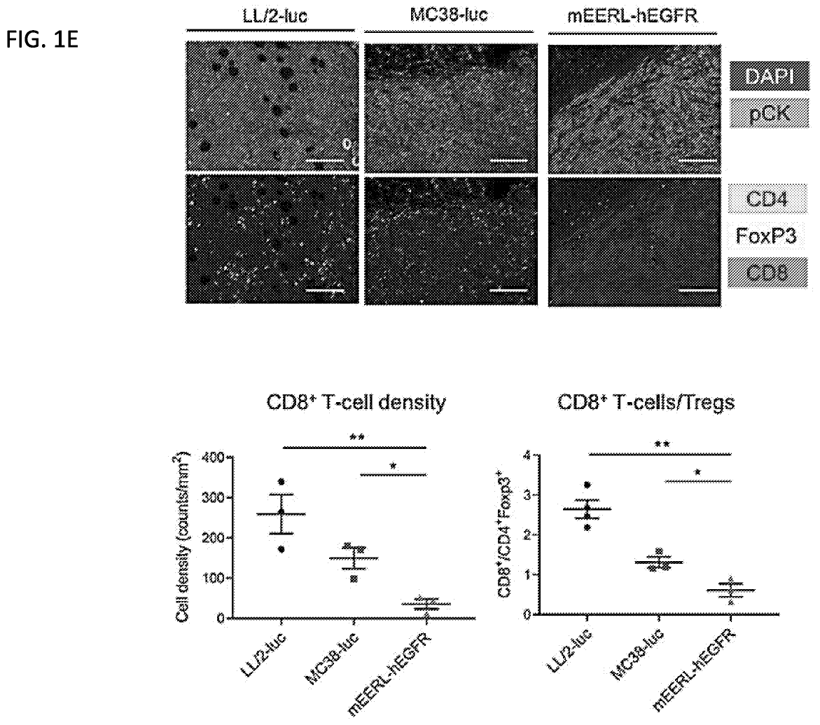

To confirm conjugates of IR700 dye and panitumumab or anti-CTLA4 antibody, SDS-page gel electrophoresis was used. Pan-IR-700 and nonconjugated control panitumumab showed a nearly identical molecular weight (FIG. 1A). Conjugates demonstrated strong fluorescent intensity, but no appreciable aggregation was detected. In anti-CTLA4 IR-700, almost the same results were demonstrated.

Based on incorporation of propidium iodine (PI) and MTT assay, cancer cell death was induced by NIR-PIT in an NIR light-dose dependent manner in mEERL-hEGFR mice exposed to pan-IR700 (FIGS. 1B, 1C). Neither NIR light alone nor panitumumab IR700 alone induced significant alterations in cell viability. Also, no significant cell damage was detected in hEGFRmEERL cells when performing CTLA4-targeted NIR-PIT. These data validated that hEGFR-targeted NIR-PIT induced target cell-specific cell death in mEERL-hEGFR cells, whereas CTLA4-targeted NIR-PIT did not affect mEERL-hEGFR cells in vitro.

### Panitumumab IR700 NIR-PIT Immediately Destroys Tumor Cells In Vivo

Cell damage in mEERL-hEGFR tumors after each NIR-PIT treatment was evaluated by histologic analysis. Tumors were harvested 1 hour after light exposure. H-E staining demonstrated swelling and vacuolation of the tumor cell in panitumumab NIR-PIT treated tumors, but no obvious change was found in CTLA4-targeted NIR-PIT-treated tumors (FIG. 1D). Thus, pathologic data demonstrated that panitumumab NIR-PIT caused the immediate death of the tumor cell but CTLA4 NIR-PIT did not.

Fewer Tumor Infiltrating T Cells in mEERL-hEGFR Tumor

To compare the immunogenicity of various tumor models, CD8⁺ tumor infiltrating lymphocytes (TIL) in three murine syngeneic tumor models, LL/2-luc, MC38-luc, and mEERL-hEGFR tumors were examined by IHC. The number of CD8⁺ TILs in mEERL-hEGFR tumors was lower than that of other two tumor models (FIG. 1E). The CD8⁺/CD4⁺FoxP3⁺ b (Treg) ratio of TILs was determined because this ratio is an indicator of a robust antitumor immune response. The intratumoral CD8⁺/Treg ratio was also significantly lower in mEERL-hEGFR tumor than in the other two tumors. These results indicated that mEERL-hEGFR tumor was poorly immunogenic compared to other syngeneic tumor models.

Tregs Express CTLA4 within the Tumor Bed

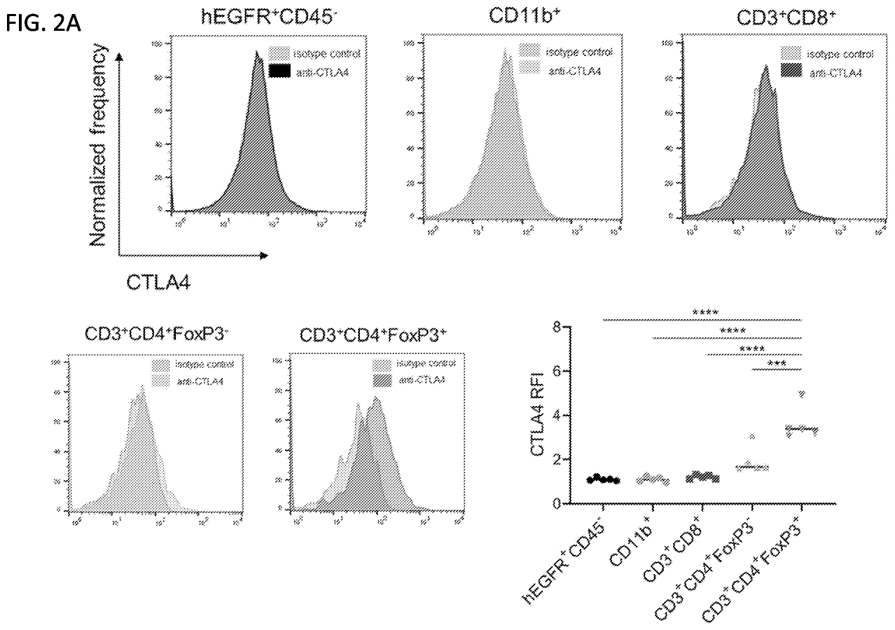

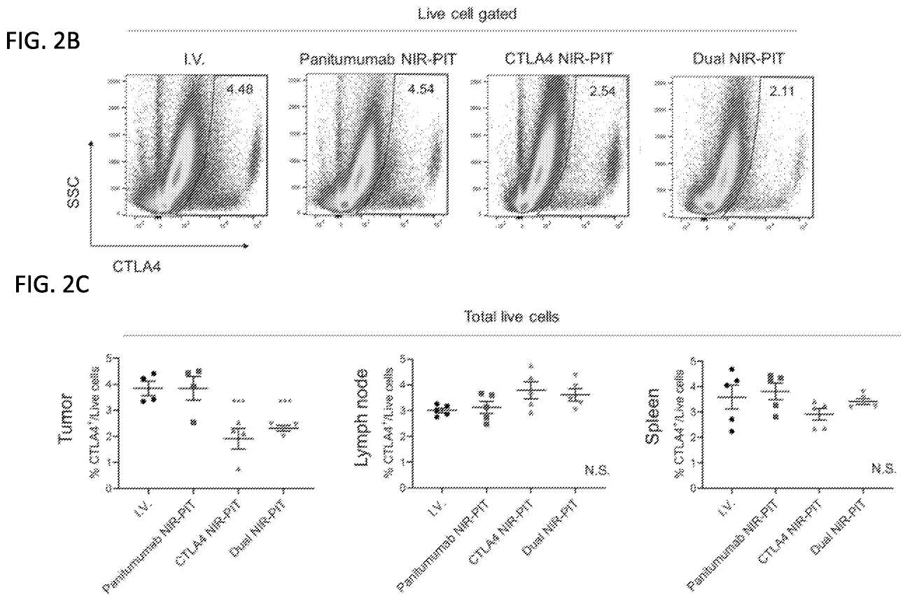

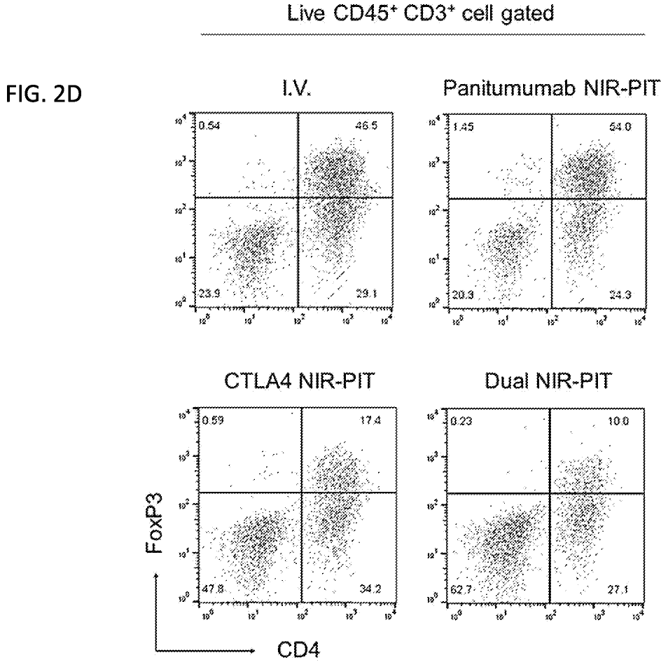

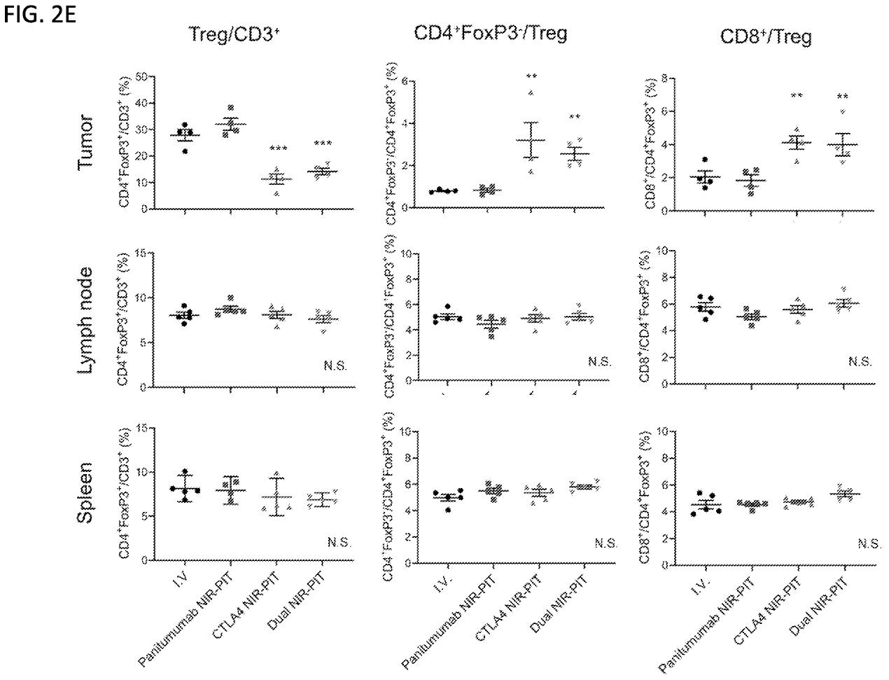

To verify the CTLA4 expressed cells in vivo, CTLA4 expression of various cell types from mEERL-hEGFR tumors was analyzed by flow cytometry. CTLA4 expression was negligible or minimal on mEERL-hEGFR cells (hEGFR⁺CD45−), myeloid cells (CD3−CD11b⁺), cytotoxic T cells (CD3⁺CD8⁺), and helper T cells (CD3⁺CD4⁺FoxP3−; FIG. 2A). In Tregs (CD3⁺CD4⁺FoxP3⁺), CTLA4 was highly expressed, and the relative fluorescence intensity (RFI) of Tregs was significantly higher than that of other cell types.

Depletion of Tregs within Tumor after CTLA4 Targeted NIR-PIT

Next, selective depletion of CTLA4 expressing cells was evaluated in tumors, tumor draining lymph nodes, and spleens after each NIR-PIT by flow cytometry (FIG. 2B). The treatment efficacy was compared among four groups; panitumumab and anti-CTLA4 antibody injection (I.V. group), pan-IR700 and anti-CTLA4 antibody injection (pan NIRPIT group), panitumumab and CTLA4-IR700 injection (CTLA4 NIRPIT group), and the combination of pan-IR700 and CTLA4-IR700 injection (dual NIR-PIT group). NIR light was applied to all groups one day after injection. CTLA4 expressing cells within the tumor were decreased in the CTLA4 NIR-PIT and dual NIR-PIT groups compared with I.V. group, but no significant changes were detected in regional lymph node or spleen (FIG. 2C). Next, the type of T cells that were selectively depleted by NIR-PIT was determined. Tregs were significantly reduced in the CTLA4 NIR-PIT and dual NIR-PIT groups (FIGS. 2D and 2E). The populations of CD4⁺FoxP3− and CD8⁺ T cells remained intact. For this result, the ratios of CD4⁺FoxP3−/Tregs and CD8⁺/Tregs were increased (FIG. 2E). In lymph node and spleen, no significant changes were observed among the four groups. These results showed that CTLA4-targeted NIR-PIT, both as monotherapy and as combination with panitumumab NIR-PIT, mainly depleted Tregs and this effect was limited to the treatment site.

In Vivo 700 nm Fluorescence Imaging of mEERL-hEGFR Cells

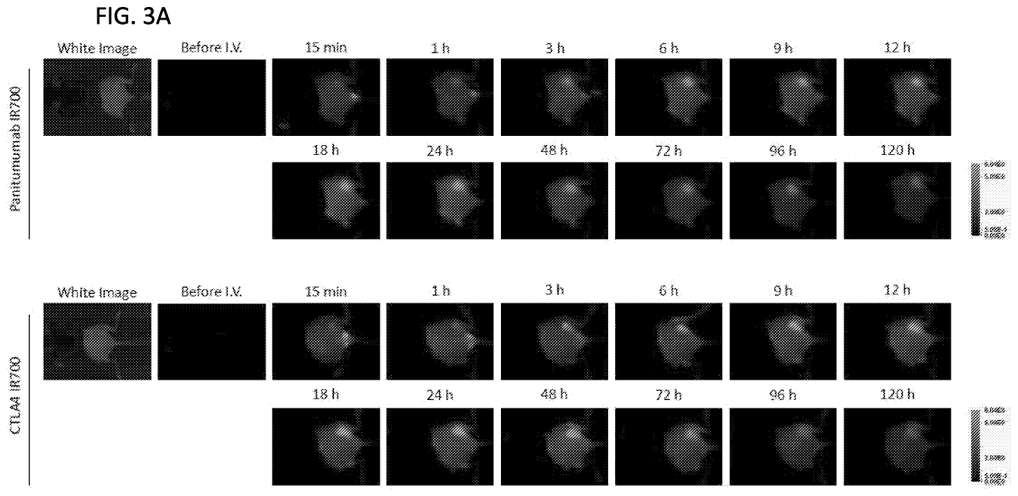

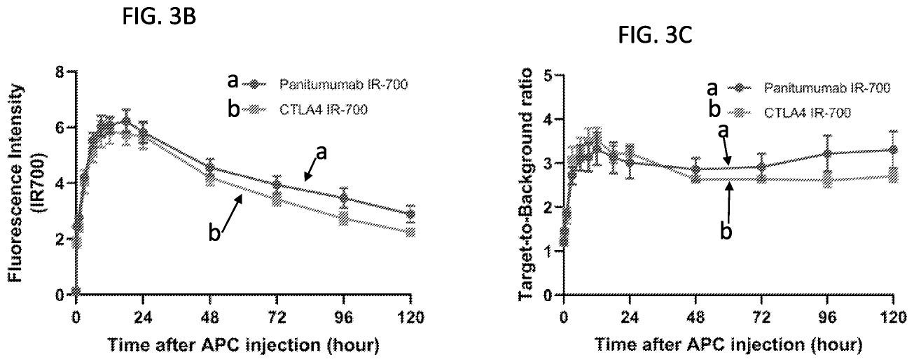

To evaluate the accumulation of each APC to the tumor, serial fluorescence images of 700 nm were obtained. The fluorescence intensity of both pan-IR700 and anti-CTLA4-IR700 in mEERL-hEGFR tumor showed peak intensities within 1 day after APC injection, and this decreased gradually over the following days (FIGS. 3A-3B). Target-to-background ratio showed a similar trend (FIG. 3C). Thus, 1 day of incubation with APC was used to obtain the maximal difference between tumor and background normal tissue.

Monotherapy Effect of CTLA4-Targeted NIR-PIT in mEERL-hEGFR Tumors

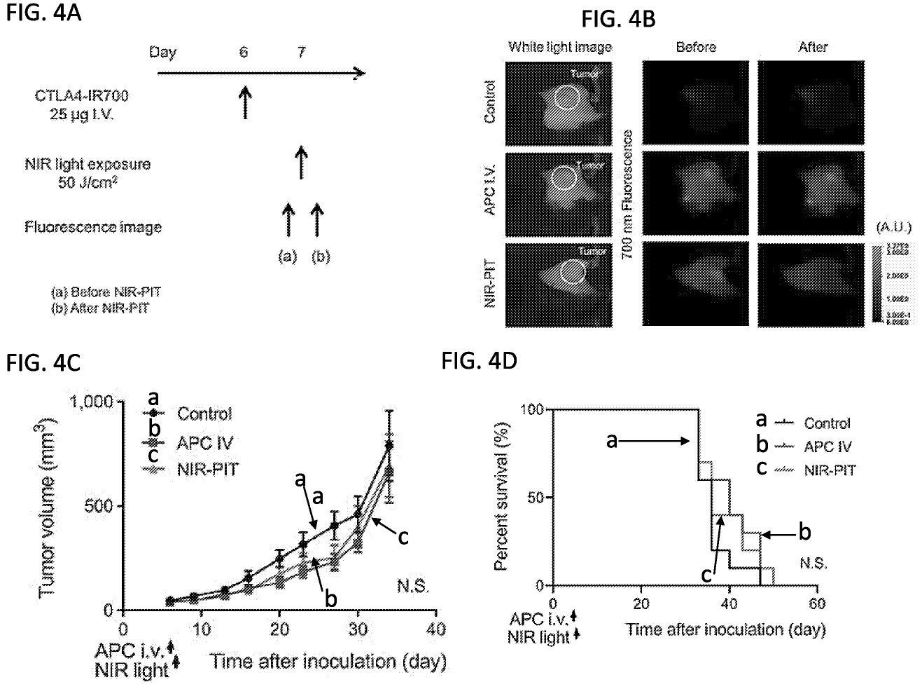

The anti-tumor effect of the monotherapy of CTLA4-targeted NIR-PIT for mEERL-hEGFR tumor was evaluated as shown in FIG. 4A. EERL tumors are not highly immunogenic. The treatment efficacy was compared among three groups; non-treatment group (control group), APC injection without NIR light irradiation (APC I.V. group), and APC injection followed by NIR light irradiation (NIR-PIT group). One day after iv injection of CTLA4 IR700, the tumors showed higher 700 nm fluorescence intensity than did the tumors with no CTLA4-IR700 (FIG. 4B). After exposure to 50 J/cm² of NIR light, IR700 fluorescence signal of tumor decreased immediately, while it was not changed in the APC I.V. or NIR-PIT groups compared with the control group, but no significant difference was detected (FIG. 4C). In the survival curve, the NIR-PIT group did not show improved survival compared with other groups (FIG. 4D). These results demonstrated that there was no significant therapeutic effect of CTLA4-targeted NIT-PIT alone in the mEERL-hEGFR allograft model.

Simultaneous Dual NIR-PIT Targeting hEGFR and CTLA4 Inhibits Tumor Growth Better than Either Single-Molecule-Targeted Therapy Alone

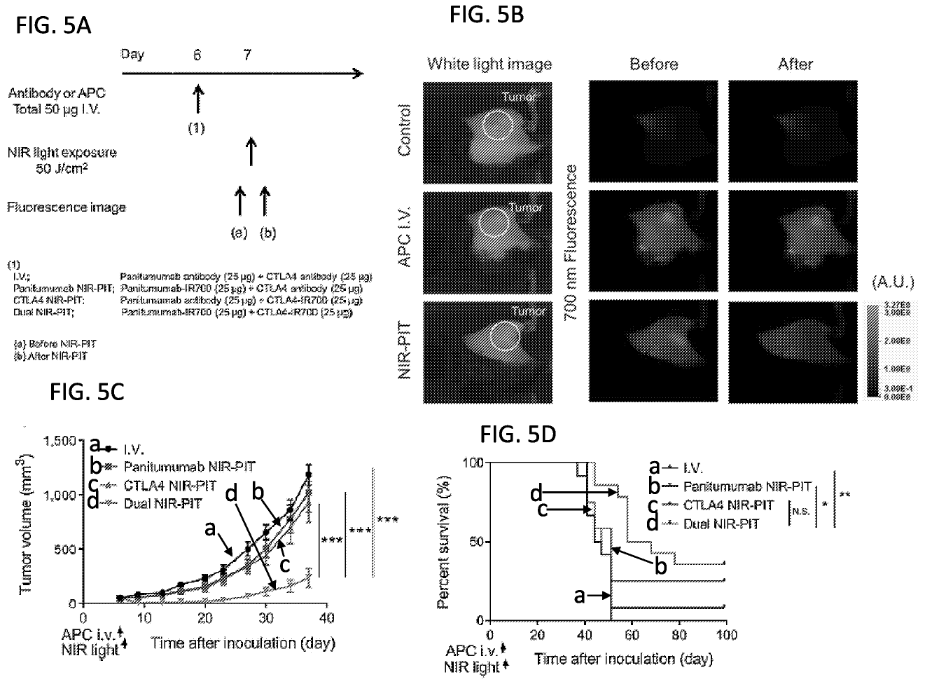

To evaluate efficacy of simultaneous dual NIR-PIT against mEERL-hEGFR tumor, in vivo experiments using pan-IR700 and anti-CTLA4 IR-700 were performed. Four tumor groups were compared; panitumumab and CTLA4 antibody (IV only), pani-IR700 and CTLA4 antibody (Panitumumab NIR-PIT), panitumumab and CTLA4 IR700 (CTLA4 NIR-PIT), and pan-IR700 and CTLA4 IR700 (dual NIR-PIT) (FIG. 5A). All groups were exposed to NIR light exposure (50 J/cm²) just once on 1 day after drug administration. Fluorescence of 700 nm at the tumor was detected in the groups injected with APCs (panitumumab NIR-PIT, CTLA4 NIR-PIT, and dual NIR-PIT groups) and this signal immediately decreased after NIR light exposure (FIG. 5B). Tumor volume in the dual NIR-PIT group was significantly decreased as compared to any other groups (p<0.001, each group vs dual PIT group) (FIG. 5C). Tumor progression in both panitumumab and CTLA4 NIR-PIT groups was not significantly decreased compared with the I.V. group. Furthermore, the dual NIR-PIT group achieved significantly prolonged survival compared with the IV-only group (p<0.01) and the panitumumab NIR-PIT group (P<0.05) (FIG. 5D). Although the survival of the dual NIR-PIT group was not significantly prolonged as compared to the CTLA4 NIR-PIT group, a larger number of the tumors was cleared in dual NIR-PIT group than in CTLA4-NIR-PIT monotherapy (38% vs. 25%).

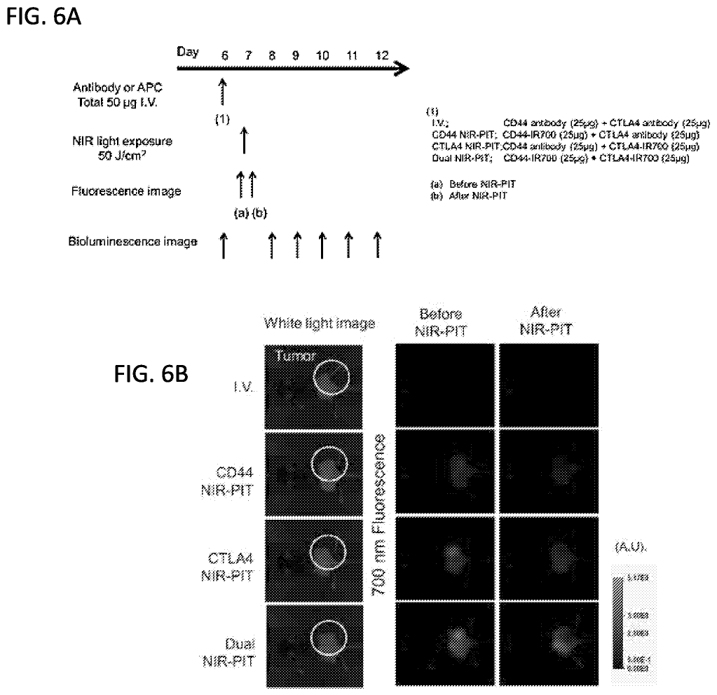

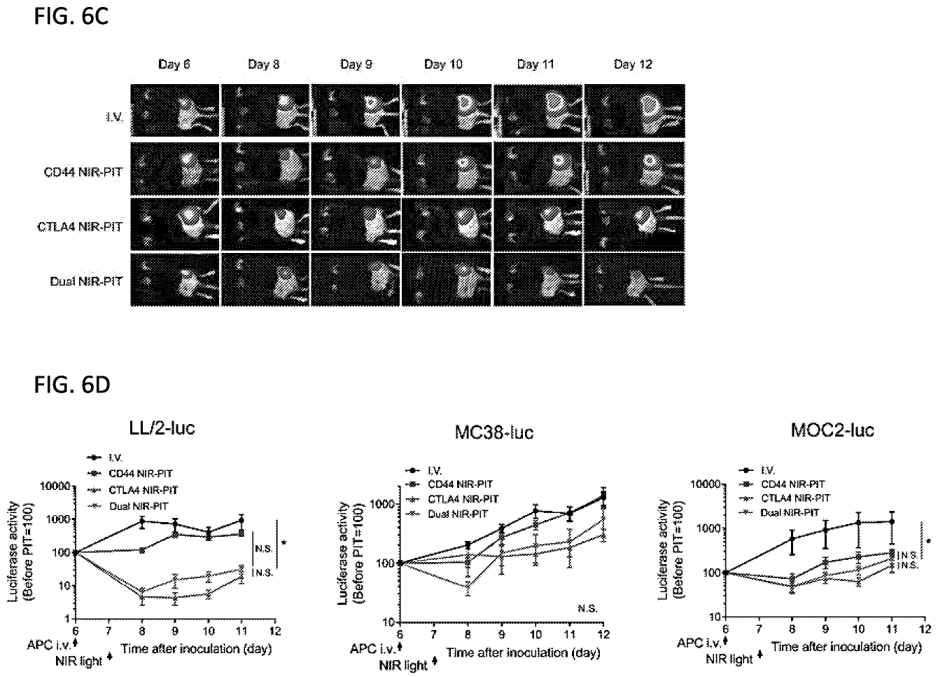

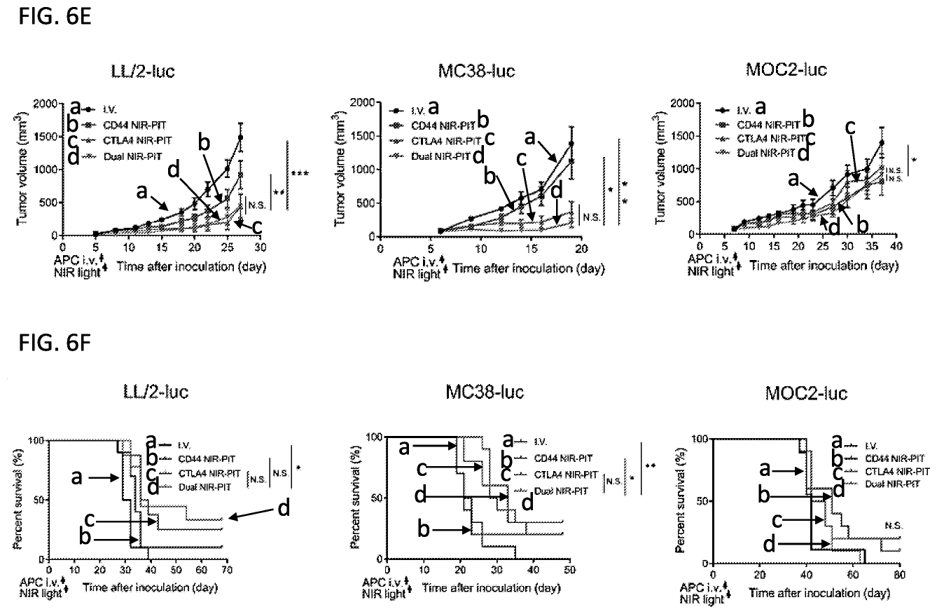

To evaluate the combined effect of CTLA4-targeted NIR-PIT and other cancer cell-targeted NIR-PIT, the efficacy of dual NIR-PIT was investigated utilizing CD44 and CTLA4 antibodies in LL/2-luc, MC38-luc, and MOC2-luc tumors (FIGS. 6A-6F). For the CTLA4/CD44 dual-targeted NIR-PIT experiment, tumor-bearing mice were randomized into four groups and intravenous injection of the mixed antibody/APC followed by NIR light exposure was performed as follows: i) mixed 25 μg anti-CD44-antibody and 25 μg anti-CTLA4-antibody (I.V. group), ii) mixed 25 μg CD44-IR700 and 25 μg anti-CTLA4-antibody (CD44 NIR-PIT group), iii) mixed 25 μg anti-CD44-antibody and 25 μg CTLA4-IR700 (CTLA4 NIR-PIT group), and iv) mixed 25 μg CD44-IR700 and 25 μg CTLA4-IR700 (Dual NIR-PIT group). The mixed antibody/APC was injected 6 days after inoculation of cancer cells in C57BL/6 mice. 24 hours after administration, NIR light (690 nm, 150 mW/cm²) was applied to tumors at 50 J/cm² in all groups. All areas outside of the tumor was covered with aluminum foil. To obtain bioluminescence images in MC38-luc, MOC2-luc, and LL/2-luc tumor-bearing mice, D-luciferin (15 mg/mL, 200 mL for MC38-luc and MOC2-luc; 3 mg/mL, 200 mL for LL/2-luc; Gold Biotechnology, St. Louis, MO, USA) was intraperitoneally injected to mice. Luciferase activity was analyzed with a BLI system (Photon Imager; Biospace Lab, Nesles la Vallée, France) using relative light units. ROIs were placed over the entire tumor. The counts per minute of relative light units were calculated using M3 Vision Software (Biospace Lab) and converted to the percentage based on those before NIR-PIT using the following formula: [(relative light units after treatment)/(relative light units before treatment)×100(%)]. CTLA4/CD44 dual NIR-PIT group significantly inhibited tumor progression compared to the I.V. group in all tumor models and prolonged the survival in LL/2-luc and MC38-luc tumors. CTLA4/CD44 dual-targeted NIR-PIT eradicated 44% and 40% of established LL/2-luc and MC38-luc tumors, respectively; however, no additional effect was observed between CTLA4-targeted NIR-PIT monotherapy and CTLA4/CD44 dual-targeted NIR-PIT in the tested tumor models.

### Dual-Targeted NIR-PIT Activates Antitumor Host Immunity

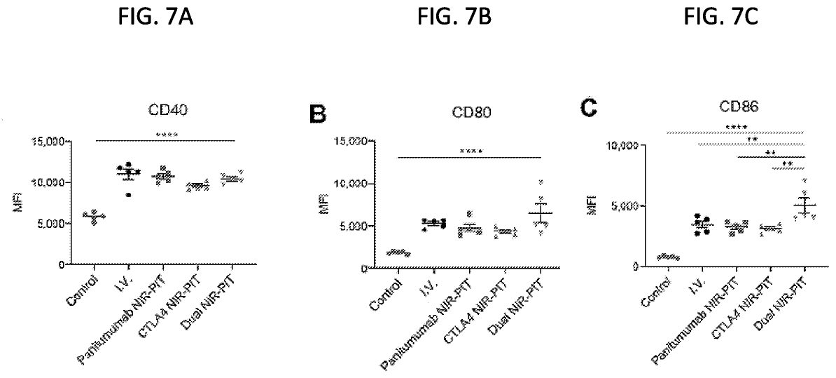

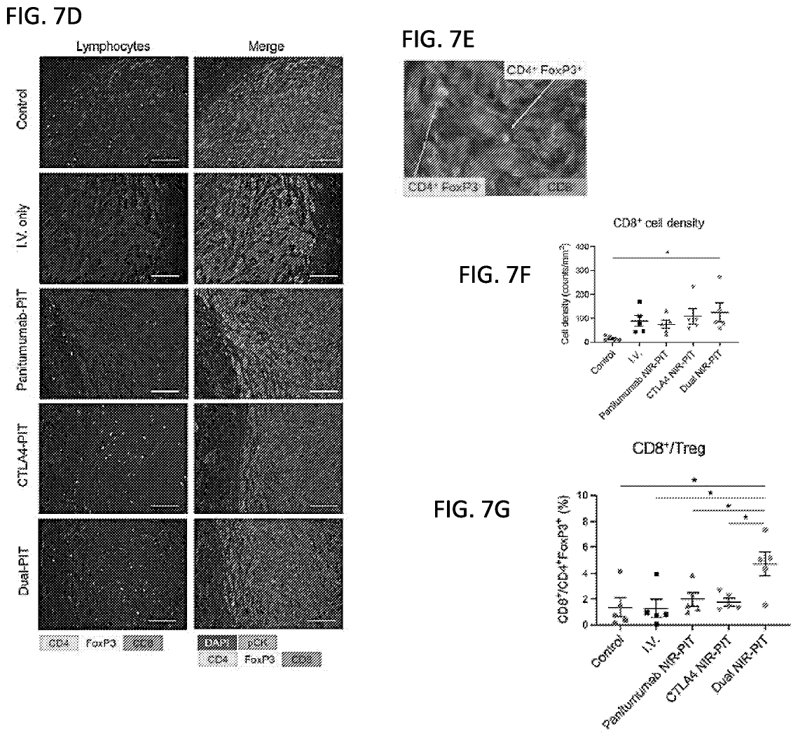

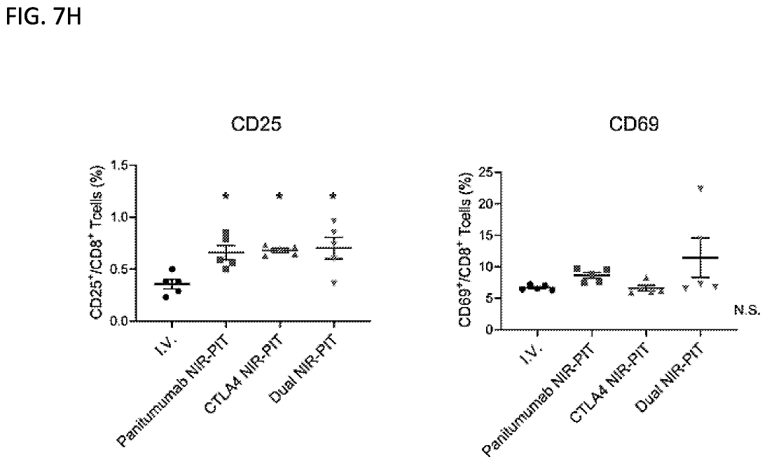

To assess the activation of dendritic cells (DCs) and CD8b T cells after each NIR-PIT, the ipsilateral inguinal lymph nodes were harvested 2 days after NIR-PIT, and analyzed by flow cytometry. The mean fluorescence intensity (MFI) of CD40 and CD80 in DCs was significantly higher in the dual NIR-PIT group compared with no treatment group (control group; FIGS. 7A and 7B). In CD86, the MFI in the dual group was higher than that of any other group (FIG. 7C). Furthermore, the percentage of CD25⁺ cells among CD8⁺ T cells within lymph nodes was significantly higher in all groups treated by NIR-PIT compared with the I.V. group (FIG. 7H). These results indicate that DC maturation and activation of cytotoxic T-cell response against the cancer cells was enhanced by activated DCs after dual-targeted NIR-PIT.

### Dual-Targeted NIR-PIT Leads to Accumulation of CD8⁺ T Cells in Tumor Tissue

To evaluate the accumulation of lymphocytes in the TME after each therapy, TILs were analyzed multiplex IHC (FIGS. 7D and 7E). CD8⁺, CD4⁺FoxP3⁻, and CD4⁺FoxP3⁺ T cells were counted for each specimen. CD8⁺ T cells had a significantly higher density in the dual NIR-PIT group compared with the control group (FIG. 7F), whereas CD8⁺/Treg ratio in the dual NIR-PIT group was significantly higher than all other groups (FIG. 7G). These results demonstrated that dual-targeted NIR-PIT reversed the immunosuppressive TME, resulting in T-cell activation, which strongly suppressed tumor progression compared with either single molecule-targeted therapy.

### Dual-Targeted NIR-PIT Results in Immunologic Memory

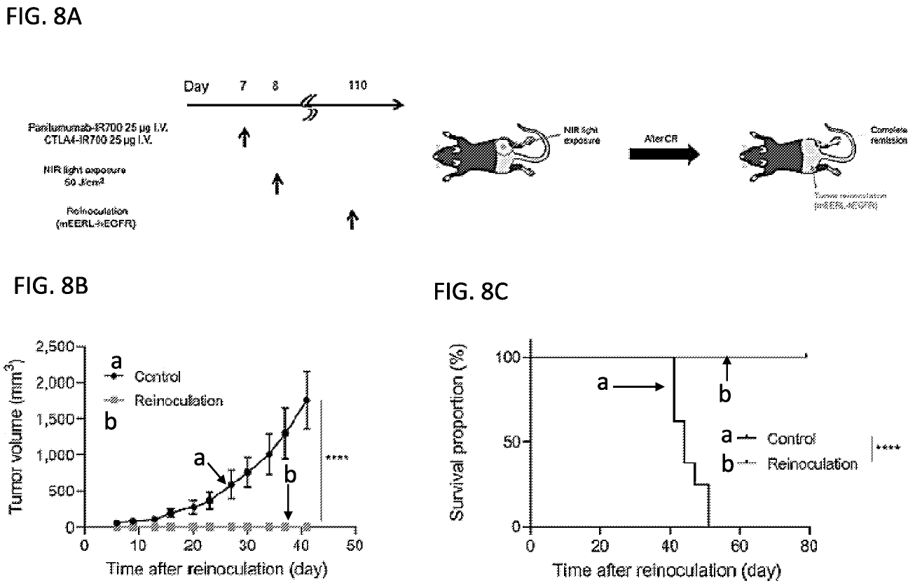

To examine immunological memory, mice whose treated mEERL-hEGFR tumors disappeared after dual NIR-PIT were re-inoculated with mEERL-hEGFR cells approximately 15 weeks after the initial NIR PIT on the contralateral dorsum (FIG. 8A). All mice that had cleared the previously inoculated tumor by dual NIR-PIT rejected the newly implanted mEERL-hEGFR cells completely, whereas no rejection was observed in control mice (FIG. 8B). In addition, all mice that had cleared the previous inoculated tumor by dual NIR-PIT survived, while all control mice died within about 50 days (FIG. 8C). This demonstrates the development of immunologic memory after dual-targeted NIR-PIT.

### Dual-Targeted NIR-PIT Demonstrates Abscopal Effect in a Bilateral Tumor Model

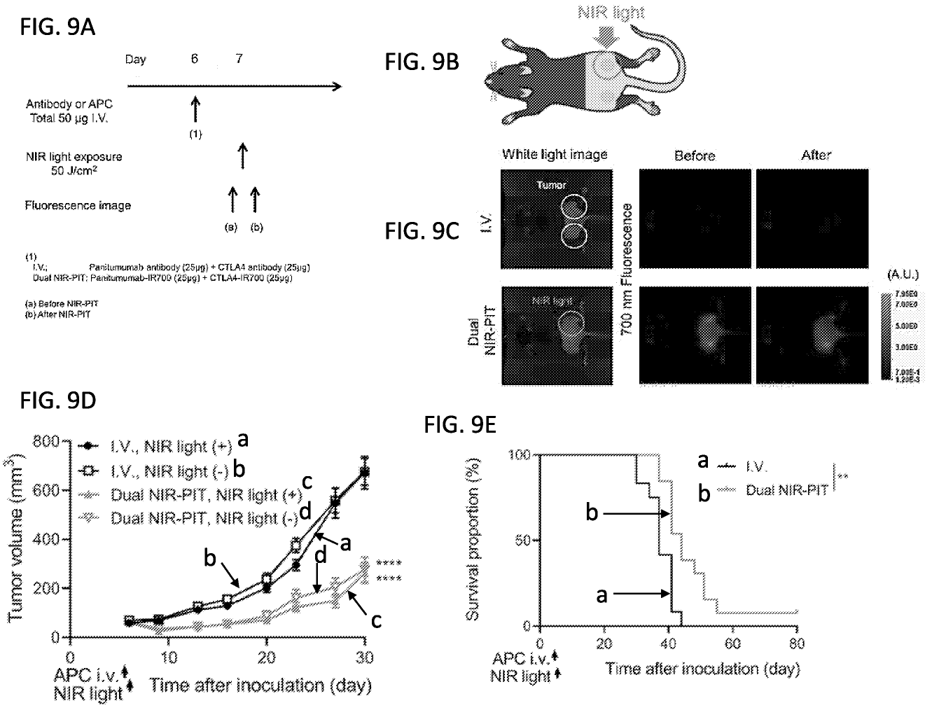

To assess the development of systemic antitumor immunity, a bilateral mEERL-hEGFR tumor model was established, and the treatment efficacy was compared between I.V. group and dual NIR-PIT group. The treatment and imaging regimen are shown in FIG. 9A. The contralateral tumors were shielded from NIR light exposure FIG. 9B). After exposure of the ipsilateral tumor to 50 J/cm² of NIR light, 700 nm fluorescence signal of the tumor with irradiation of NIR light [NIR light (+)] was decreased in the dual NIR-PIT groups, while 700 nm fluorescence in the contralateral tumor [NIR light (−)] was unchanged (FIG. 9C). Tumor growth was significantly suppressed in the dual NIR-PIT group compared with the I.V. group for not only NIR light+) tumors but also NIR light (−) tumors [P<0.0001 (both sides), vs. I.V. group; FIG. 9D]. No significant difference was observed between the NIR light (+) and NIR light (−) tumor in each group. The survival of the dual NIR-PIT group was also significantly prolonged compared to I.V. group (FIG. 9E). Furthermore, complete remission of both side tumors using a one-sided dual targeted NIR-PIT was achieved in 1 of 10 mice.

### Discussion

Simultaneous CTLA4/hEGFR dual-targeted NIR-PIT showed a stronger tumor response than either treatment alone even at the same antibody doses. The CD8+/Treg ratio at 3 hours and 7 days after dual-targeted NIR-PIT was significantly increased compared with other therapies. Furthermore, abscopal effects were observed in a model of bilateral tumors after dual-targeted NIR-PIT. These results indicate that dual-targeted NIR-PIT can alter the immunosuppressive TME, resulted in strong synergistic effects on tumor growth suppression.

Administration of unconjugated CTLA4 and hEGFR antibody monotherapy did not demonstrate any therapeutic effects (FIGS. 4-6). Indeed, because there was no change in the number of Tregs in the spleen (FIGS. 2C and D), it is unlikely that anti-CTLA4 antibody reduced the number of Tregs because less than half of the therapeutic antibody dose was used (Nagaya et al., Cancer Immunol Res 2019; 7:401-13; Maruoka et al., Cancer Immunol Res 2020; 8:345-55; Okada et al., Bioconjug Chem 2019; 30:2624-33). However, a sizeable therapeutic effect was observed with dual-targeted NIR-PIT using the lower dose of anti-CTLA4 antibody. Serious autoimmune adverse effects associated with cancer immunotherapies including ICIs were frequently reported with anti-CTLA4 antibody (ipilimumab) in humans (Hodi et al., N Engl J Med 2010; 363:711-23; Weber et al., Lancet Oncol 2015; 16:375-84; Weber et al., N Engl J Med 2017; 377:1824-35; Robert et al., N Engl J Med 2011; 364:2517-26; Quirk et al., Transl Res 2015; 166:412-24). Ipilimumab induces more frequent immune-related adverse effects (irAE) than other ICIs particularly causing gastrointestinal symptoms in about 40% of patients when given at a dose of 3 mg/kg for melanoma therapy (Dougan et al., Support Care Cancer 2020; 28: 6129-43; Dougan M. Front Immunol 2017; 8:1547; Eggermont et al., N Engl J Med 2016; 375:1845-55; Puzanov et al., J Immunother Cancer 2017; 5:95.). The irAEs of ipilimumab or pembrolizumab, which blocks PD-L1, are reportedly dose dependent (Dougan et al., Support Care Cancer 2020; 28: 6129-43; Puzanov et al., J Immunother Cancer 2017; 5:95; Maughan et al., Front Oncol 2017; 7:56). Therefore, low-dose administration of ICIs might minimize irAE. Thus, the dual-targeted NIR-PIT has a great advantage not only because of its superior therapeutic effects compared with ICIs but also because the antibody doses may reduce adverse effects.

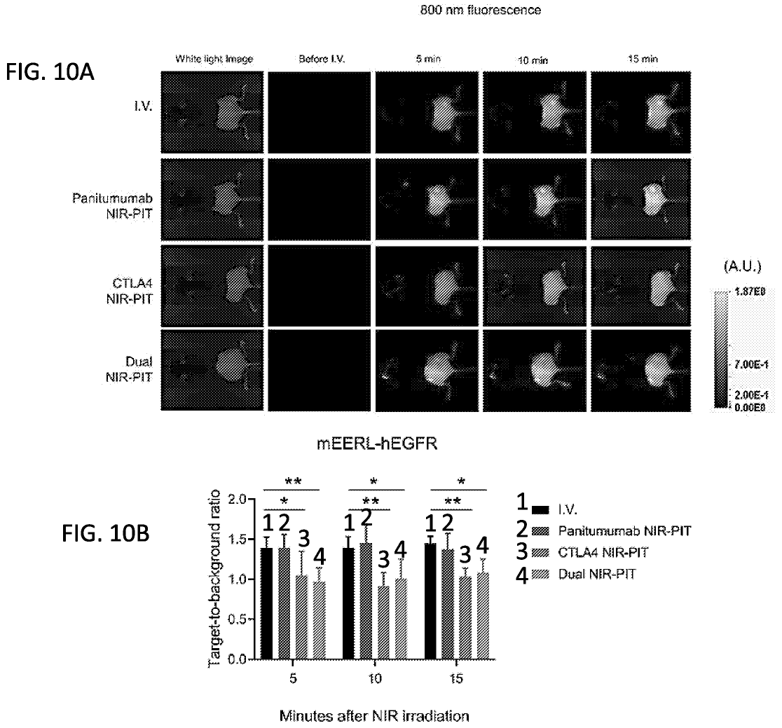

CTLA4 is expressed in FoxP3b Tregs and various other cells. Because the endocytosis of CTLA4 is extremely rapid, it is difficult to capture the expression of CTLA4 on the cell surface. Nevertheless, CTLA4-targeted NIR-PIT could still deplete Tregs within tumors. Furthermore, myeloid-derived suppressor cells and several kinds of cancer cells also expressed CTLA4 on the cell membrane (Yu et al., Oncoimmunology 2016; 5:e1151594; Pico de Coana et al., Cancer Immunol Immunother 2014; 63:977-83; Chaudhary et al., Vaccines 2016; 4:28). These cells could also be depleted by CTLA4-targeted NIR-PIT, which could further enhance antitumor host immunity. In addition, CTLA4-targeted NIR-PIT was reported to suppress intratumoral blood perfusion (Okada et al., Adv Ther 2021; n/a:2000269; Kurebayashi et al., Cancer Res 2021; 81:3092-104). Intratumoral blood flow reduction was observed after dual-targeted NIR-PIT similar to the monotherapy of CTLA4-targeted NIR-PIT (FIGS. 10A, 10B). This early vascular effect might also contribute to the overall therapeutic effects.

CD25-targeted NIR-PIT also induced selective Treg depletion; however, because CD25 is the IL2 receptor, the presence of residual APC after the light exposure might block IL2/IL2R binding on activated effector cells resulting in their inhibition. CTLA4-targeted NIR-PIT might be better than CD25-targeted NIR-PIT because CTLA4 antibody does not block IL2 binding and CTLA4-IR700 would block the CTLA4 immune checkpoint pathway resulting in enhancement of host antitumor immunity (Okada et al., Adv Ther 2021; n/a:2000269).

Dual-targeted NIR-PIT utilizing CTLA4 combined with CD44 as the cancer targeting agent was also evaluated, but no synergistic effects were observed in CD44 expressing LL/2-luc, MC38-luc, and MOC2-luc tumor models (FIGS. 6A-6F). There may be several reasons why the CTLA4/CD44 combination in these models was less effective than CTLA4/hEGFR in mEERL-hEGFR model. First, MC38-luc and LL/2-luc tumors are highly immunogenic tumors. Host tumor immunity was enhanced by CTLA4-targeted NIR-PIT alone possibly because the tumors were already highly infiltrated with lymphocytes before any treatment (FIG. 1E). In contrast, mEERL-hEGFR tumor was thought to be a poorly immunogenic tumor as indicated by the low infiltration of T cells (FIG. 1E) and the lack of therapeutic effect of CTLA4-targeted NIR-PIT by itself. CTLA4/hEGFR dual targeted NIR-PIT inhibited tumor progression in the mEERL-hEGFR allograft model and enhanced DC maturation and activation of T-cell response (FIGS. 5 and 7). Another reason would be off-target cell killing. MOC2-luc tumor was also poorly immunogenic tumor, however, the synergistic effect of CTLA4/CD44 dual-targeted NIR-PIT was not seen in this model. CD44 is expressed not only on cancer cells but also on some immune cells, such as, effector T cells and memory T cells (Govindaraju et al., Matrix Biol 2019; 75-76:314-30). CD44-positive subset of CD8+ T cells was depleted by CD44-targeted NIR-PIT (Maruoka et al. Vaccines 2020; 8:528). Therefore, CD44-targeted NIR-PIT could also damage effector T cells, resulting in a weakened antitumor effect. On the other hand, NIR-PIT targeting hEGFR kills only hEGFR-expressing cancer cells without damaging the host immune cells which do not express hEGFR. Thus, CTLA4/hEGFR dual-targeted NIR-PIT in the mEERL-hEGFR tumor model successfully induced antitumor immune activation by targeting only cancer cells and amplified the effect by eliminating CTLA4 expressing cells, which are generally immunosuppressive.

In conclusion, the data demonstrate that CTLA4/hEGFR dual-targeted NIR-PIT successfully depleted cancer cells and CTLA4-expressing cells in the intratumoral tissues and had a significant impact on cell growth, surpassing that of either agent alone. This combined NIR-PIT approach can enhance therapeutic effects, especially when the efficacy of NIR-PIT monotherapy is unsatisfactory.

### Example 3

### PD-L1-IR700+IL-15 Preconditioning Provides Synergistic Anti-Tumor Effect in Mice

The materials and methods described in Example 1 were used to demonstrate the effect of PD-L1-antibody-IR700 conjugates in PD-L1-targeted NIR-PIT, except that an anti-mouse CD274 antibody from Bio X Cell (West Lebanon, NH, USA) was used (10F.9G2 monoclonal antibody) instead of anti-mouse CTLA4 antibody (9D9). The methods further included pre-conditioning with IL15 prior to administration of the PD-L1-antibody-IR700 conjugate.

IR700 Conjugated with Anti-PD-L1 (CD274)

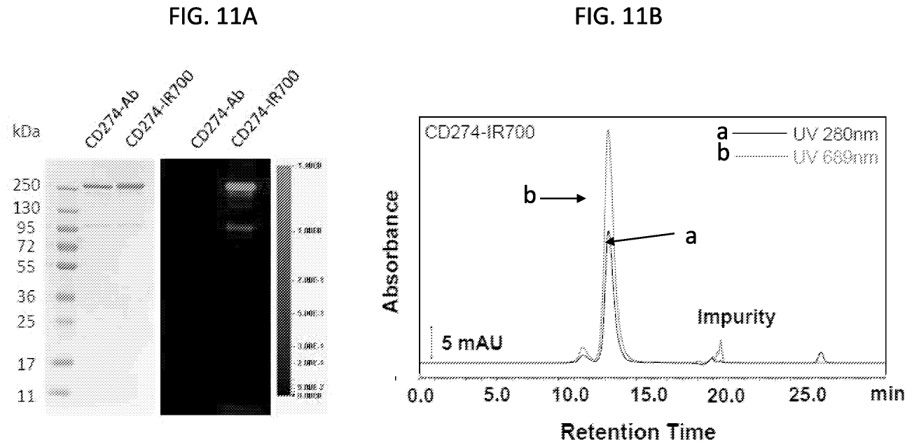

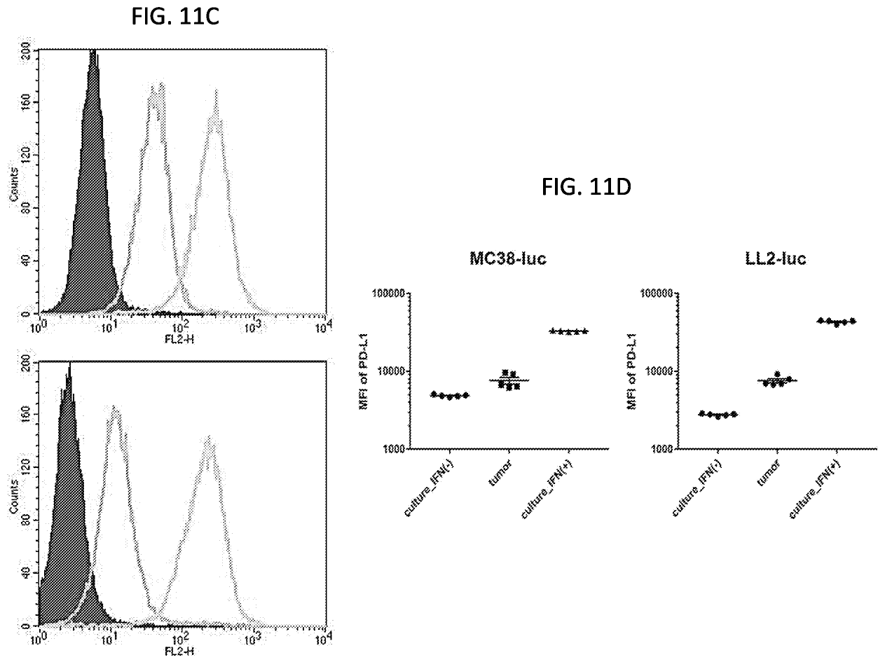

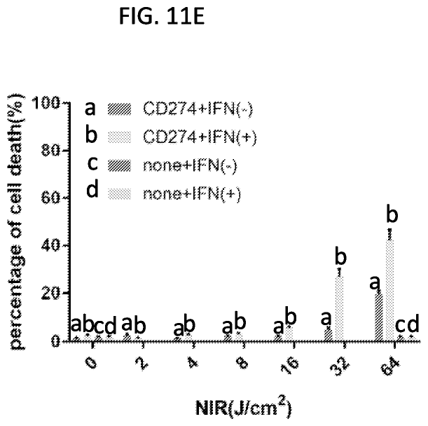

To synthesize the APC, IR700 was conjugated to anti-PD-L1 antibody (clone 10F.9G2) and the conjugate (anti-PD-L1-IR700 or CD274Ab-IR700) was analyzed by SDS-PAGE. Anti-PD-L1 and anti-PD-L1-IR700 had approximately the same molecular weight but only anti-PD-L1-IR700 had a fluorescence of 700 nm (FIG. 11A). The APC was also evaluated with SEC. The majority of the protein, which was detected with the absorption of 280 nm as the main peak (eluting at 11.5 min), had 689-nm absorbance and fluorescence (excitation, 689 nm; emission, 700 nm) (FIG. 11B). These results verified the successful conjugation of the APC.

### Expression of PD-L1 in Cancer Cells and Immune Cells

Flow-cytometric analysis of PD-L1 expression on cancer cell lines was performed. Representative histograms of MC38-luc (top) and LL2-luc (bottom) are shown in FIG. 11C. Flow-cytometric analysis of PD-L1 expression on cancer cell lines MC38-luc and LL2-luc was obtained from cell culture with or without interferon gamma and in vivo tumor. As shown in FIG. 11A, interferon gamma conditionally enhanced the expression of PD-L1. Microscopic images before and after PD-L1 targeting NIR-PIT to cultured cancer cells in vitro demonstrated that the cells, which changed in shape, are crushed. In vitro PD-L1 targeting NIR-PIT showed better cell killing against interferon gamma treated MC38-luc cells (FIG. 11E).

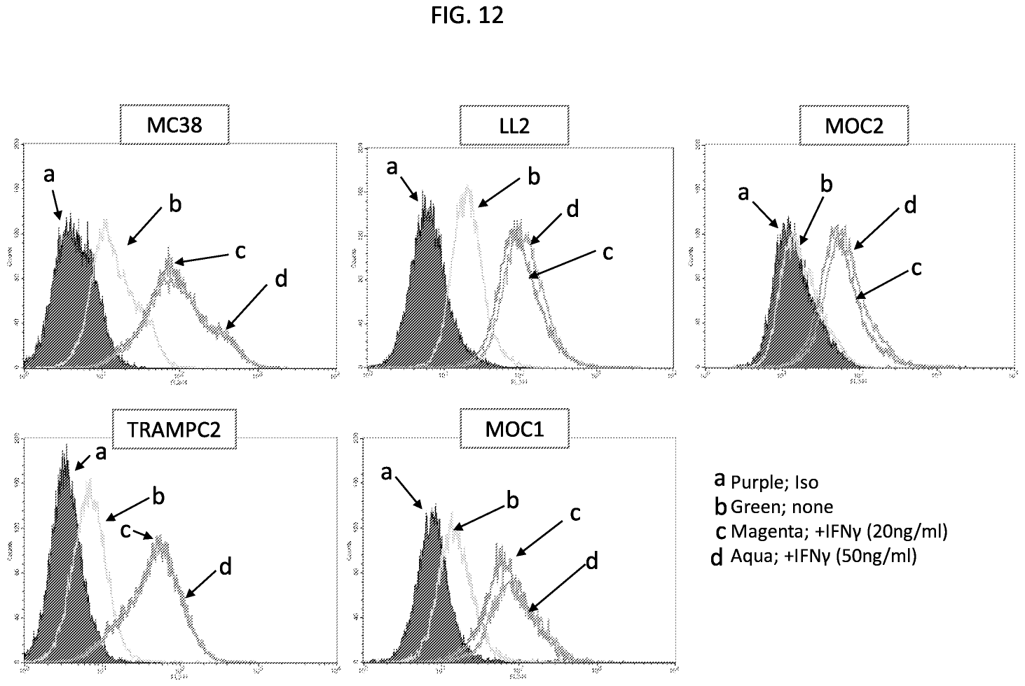

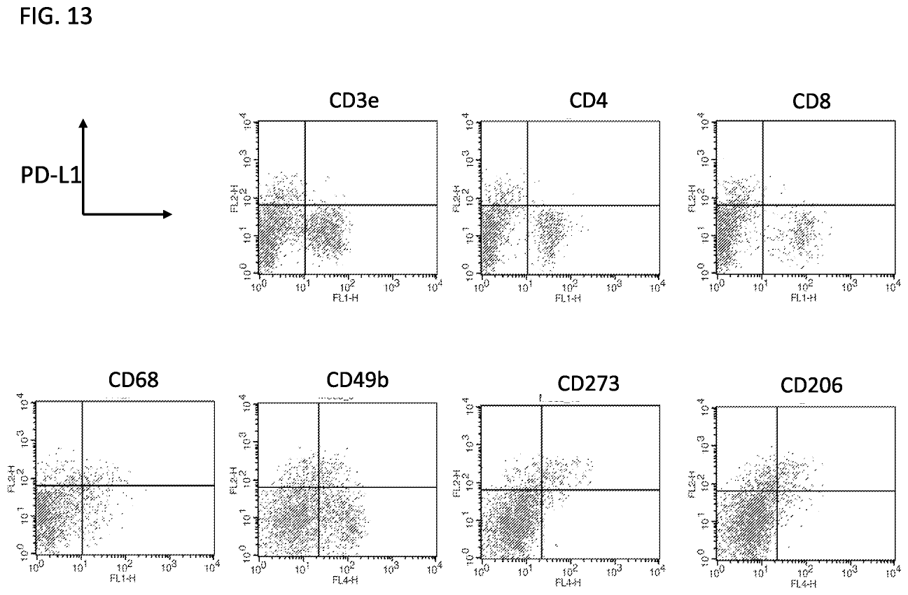

As shown in FIG. 12, PD-L1 expression increases in cancer cells flowing exposure to IFN-gamma. As shown in FIG. 13, PD-L1 is expressed in tumor infiltrating immune cells.

Treatment of Cancer with Anti-PD-L1-IR700 and IL-15

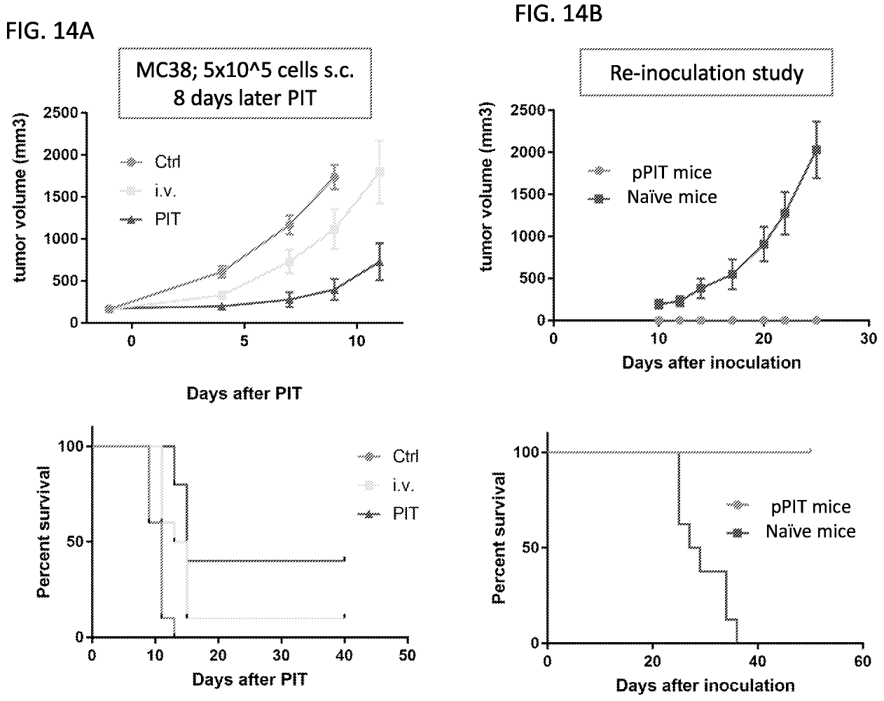

Anti-PD-L1-IR700 was administered to mice bearing MC38-luc tumors. Tumors were established by administration of 5×10⁵ MC38 cells s.c. Eight days later, anti-PD-L1-IR700 (or control IgG-IR700) was administered i.v. and the tumor subjected to NIR light (50 J/cm2) (PIT) or not. As shown in FIG. 14A (left panels), anti-PD-L1-IR700 with NIR light significantly reduced tumor volume and increased survival, to a greater extent than anti-PD-L1-IR700 alone with NIR light. Mice were subsequently administered an additional dose of MC38 cells, and the effect on tumor volume and survival, measured. As shown in FIG. 14B (right panels), mice previously treated with anti-PD-L1-IR700 had no tumor recurrence and survived, in contrast to naïve mice.

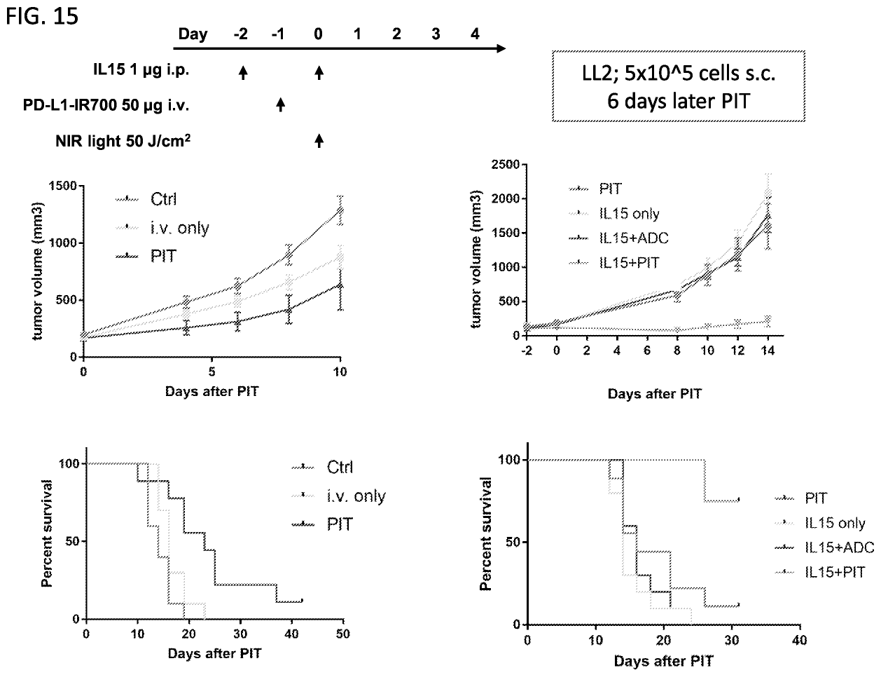

The effect of IL15 preconditioning in combination with anti-PD-L1-IR700 PIT was examined as shown in FIG. 15. Tumors were established in mice by administration of 5×10⁵ LL2 cells s.c. Four days later, IL15 (1 ug i.p.) was administered, followed one day later with anti-PD-L1-IR700 (or vehicle control), followed with or without NIR light (50 J/cm2) another day later. As shown in FIG. 15 (left panels), anti-PD-L1-IR700 in combination with NIR light significantly reduced tumor volume and increased survival, to a greater extent than anti-PD-L1-IR700 without NIR light. As shown in FIG. 15 (right panels), addition of IL-15 precondition substantially reduced tumor volume and increased survival, to a greater extent than anti-PD-L1-IR700 with NIR.

### Example 4

### Materials and Methods

This example describes materials and methods used to obtain the results in Examples 5-11.

### Reagents

A water soluble, silica-phthalocyanine derivative IRDye700DX NHS ester was from LI-COR Biosciences (Lincoln, NE). Panitumumab, a fully humanized IgG2 monoclonal anti-body (mAb) directed against EGFR, was from Amgen (Thousand Oaks, CA). Anti-mouse CTLA4 antibody (Clone; 9D9) was from Bio X Cell (West Lebanon, NH). L-sodium ascorbate (L-NaAA) and L-cysteine were from MilliporeSigma (Burlington, MA). Sodium azide (NaN3) was from MP biomedicals, LLC (Solon, OH).

### Synthesis

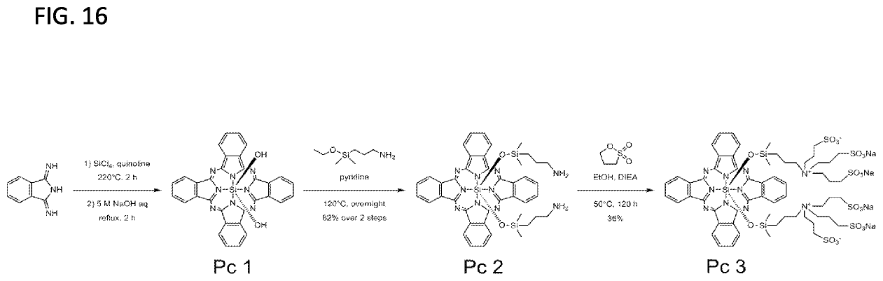

The compounds were prepared by a slight modification of a previously reported method (FIG. 16) (Sato et al., ACS central science 2018, 4:1559-1569). General chemicals were of the best grade available, supplied by FUJIFILM Wako Pure Chemical Corporation, Tokyo Chemical Industries Co, Ltd., KANTO CHEMICAL Co., INC. and Sigma-Aldrich Japan K. K., and were used without further purification. 1H NMR spectra were recorded on a JNM-ECX400P or JMN-ECS400 (JEOL Ltd, Tokyo, Japan) instrument at 400 MHz and are reported relative to deuterated solvent signals.

### Silicon Phthalocyanine Dihydroxide (Pc 1)

Silicon tetrachloride (1.93 g, 11.4 mmol) and 1,3-diiminoisoindoline (1.10 g, 7.59 mmol) were dissolved in quino-line (13 mL), and the mixture was refluxed for 2 h under an argon atmosphere. After the mixture was cooled to room temperature (RT), 5 M NaOH aq (10 mL) was added, and the mixture was refluxed for 2 h. The product was recovered by filtration, washed with MeOH and dried in vacuo (970 mg, 1.69 mmol). The product was used for next reaction without further purification.

### Bis(3-Aminopropyldimethylsilyl Oxide) Silicon Phthalocyanine (Pc 2)

Pc 1 (500 mg, 0.87 mmol) and 3-aminopropyldimethylethoxysilane (1.12 g, 6.96 mmol) were dissolved in pyridine (250 mL), and the mixture was refluxed overnight under an argon atmosphere, concentrated by rotary evaporation. The residue was diluted, filtered, washed with a H₂O-ethanol solution (2:1), and dried in vacuo (650 mg, 0.807 mmol, yield 82% (2 steps)). 1H NMR (400 MHz, CD30D): δ −2.86 (s, 12H), −2.33 to −2.27 (m, 4H), −1.28 to −1.19 (m, 4H), 1.18 (t, J=7.2 Hz, 4H), 8.35 (dd, J=5.7, 2.8 Hz, 8H), 9.66 (dd, J=5.7, 2.8 Hz, 8H).

### Bis{3-[Tris(3-Sulfopropyl)]Ammoniopropyldimethylsilvloxide} Silicon Phthalocyanine (Pc 3)

Pc 2 (300 mg, 0.373 mmol), 1,3-propansultone (2.27 g, 18.6 mmol), and N,N-diisopropylethylamine (DIEA, 4.82 g, 37.4 mmol) were dissolved in EtOH (15 mL), and the mixture was stirred at 50° C. for 120 h under an argon atmosphere. The product was purified by an high performance liquid chromatography (HPLC) system (Shimadzu Co., Kyoto, Japan) with a re-verse-phase column Inertsil ODS-3 (10 mm×250 mm) (GL Sciences Inc., Tokyo, Japan), using eluent A (H₂O, 0.1 M tri-ethylammonium acetate (TEAA)) and eluent B (99% MeCN, 1% H₂O) (A/B=80/20 to 50/50 in 15 min, 50/50 to 0/100 in 5 min). The product was desalted with a Sep-Pak C18 cartridge (Waters Corporation, Milford, MA) and cation-exchange resin, affording Pc 3 (216 mg, 0.132 mmol, yield 36% as a sodium salt). 1H NMR (400 MHz, CD30D): 6-2.79 (s, 12H), −2.12 to −2.18 (m, 4H), −0.88 to −0.98 (m, 4H), 1.75-1.66 (m, 12H), 2.05-1.98 (m, 4H), 2.80-2.73 (m, 24H) 8.52 (dd, J=5.7, 3.0 Hz, 8H), 9.79 (dd, J=5.7, 3.0 Hz, 8H).

### Synthesis of IR700-Conjugated Bovine Albumin, Panitumumab, Anti-CD44 Antibody, and Anti-CTLA4 Antibody

Bovine albumin (1 mg, 15.0 nmol), panitumumab (1 mg, 6.8 nmol), anti-CD44 and anti-CTLA4 antibodies (1 mg, 6.7 nmol) were incubated with fivefold molar excess of IR700 NHS ester (Albumin; 146.9 μg, 75.2 nmol, panitumumab; 66.9 μg, 34.2 nmol, anti-CD44 and anti-CTLA4 antibodies; 65.1 μg, 33.3 nmol 10 mmol L⁻¹ in DMSO) in 0.1 mol L⁻¹ Na2HPO4 (pH 8.5) at RT for 1 h. The mixture was purified with a Sephadex G25 column (PD-10; GE Healthcare, Piscataway, NJ, USA). The concentration of IR700 was determined with absorption at 689 nm using UV-Vis (8453 Value System; Agilent Technologies, Santa Clara, CA, USA). The protein concentration was confirmed with Coomassie Plus protein assay kit (Thermo Fisher Scientific, Rockford, IL, USA) by measuring the absorption at 595 nm. SDS-PAGE was performed as a quality control for each conjugate. Panitumumab-IR700 is abbreviated as pan-IR700, albumin-IR700 as alb-IR700, anti-CD44-IR700 as CD44-IR700, and anti-CTLA4-IR700 as CTLA4-IR700.

700 nm Fluorescence Evaluation of Pc 3 or mAb-IR700 after NIR Light Exposure In Vitro

25 μM Pc 3 or 3 μM pan-IR700 solution with 0, 0.1, 0.5, 1.0, and 10 mM L-NaAA or L-Cysteine in PBS buffer (pH 7.5) were pre-pared. For the analysis mixed with NaN3, 3 μM mAb-IR700 solution with 1 mM L-cysteine and/or 1 mM L-NaAA adding different concentration of NaN3 (0, 0.01, 0.1, 1, 10, or 100 mM) diluted by PBS was prepared. Pan-IR700 was exposed to NIR light (690 nm, 150 mW cm-2, 50 J cm-2) in each condition (0, 5, 10, 25, 50, 100, 150, and 200 J) with an ML7710 laser system (Modulight, Tampere, Finland). Before and after each irradiation, the 700 nm fluorescence intensity was acquired by a fluorescence imager (Pearl Imager, LI-COR Bioscience, Lincoln, NE, USA). Pearl Cam Software (LI-COR Biosciences) was used for analyzing fluorescence. The same regions of interest (ROIs) were put on the solution in each tube, and then mean 700 nm fluorescence intensity was measured. The appearance of the tube before and after NIR light irradiation was imaged. All the experiments were carried out at RT.

### Electron Spin Resonance (ESR) Spectroscopy

Thirty microliters of 0.5 mM IR700 in 100 mM phosphate buffer (pH 7.0) without or with 10 mM L-cysteine or 10 mM L-NaAA were filled into a gas-permeable polymethylpentene (TPX™) tube (0.76 mm I.D.×1.0 mm O.D.×60 mm long, Toho Kasei Sangyo Co., Ltd., Tokyo, Japan), which were then sealed at both ends and this TPX tube was placed in natural quartz ESR tube (5 mm O.D.×250 mm long, S-5-EPR-250S, Norell Inc., NC). The ESR sample tube was set in a cylindrical TE011 mode cavity (JEOL), and argon gas was allowed to flow into the ESR sample tube through a long capillary glass tube to remove oxygen from the sample. After 5 min of ventilation with argon gas, the ESR sample tube was sealed and X-band CW-ESR measurements were performed at ambient temperature using a JEOL-RE1X spectrometer (JEOL, Tokyo, Japan). ESR scan (scan time: 8 min/10 mT) was started from the low magnetic field side at the same time as irradiation of NIR light started. The ESR parameters were as follows: incident microwave; 10 mW power, microwave frequency; 9.150 GHz, modulation frequency; 100 kHz, field modulation amplitude; 0.1 mT, time constant; 0.3 sec, scan range; 325.17±5 mT, and receiver gain; 5.0×102. The line width, intensity, and g-value of ESR signal were estimated using the signal of a co-mounted Mn2+ marker (ES-DM1, JEOL) in cavity and a Win-Rad software (Radical Research, Tokyo, Japan).

HPLC Analysis of Cystine Formation from Cysteine

The solution of 0.25 mM IR700 in 10 mM sodium phosphate buffer (pH 7.0) containing 1 mM L-cysteine was prepared in a sealed cuvette and argon was bubbled through the rubber septum cap of the cuvette for 20 min. The deoxygenated solution was then irradiated with a laser (MLL-III-690, Changchun New Industries Optoelectronics Technology Co. Ltd, Changchun, China) (690 nm, 1 W cm-2) for 1 h. The solution without argon bubbling was also irradiated as above. The irradiated solutions were filtered with Millex-LH filter (pore size: 0.45 μm, hydrophilic, PTFE, Merck Millipore, US). The solutions were analyzed by an HPLC system (Shimadzu Co., Kyoto, Japan) with a reverse-phase column Inertsil ODS-3 (4.6 mm×250 mm) (GL Sciences Inc., Tokyo, Japan) using eluent A (H₂O, 0.1% trifluoro-acetic acid) and eluent B (99% MeCN, 1% H₂O) (A/B=99/1) at a flow rate of 1 mL min⁻¹. The detection wavelength was 202 nm. The solutions were also co-injected with authentic compounds to identify L-cysteine and L-cystine peak.

### Cell Culture

Luciferase expressing murine cancer cell lines, A431 GFP-luc (EGFR expressing epidermoid cancer), MDAMB468 GFP-luc (EGFR expressing breast cancer), MC38-luc (colon cancer), LL/2-luc (lung cancer), and MOC2-luc (oral cancer) were used. A431 GFP-luc, MDAMB468 GFP-luc, MC38-luc, and LL/2-luc cells were cultured in RPMI1640 medium (Thermo Fisher Scientific) supplemented with 10% fetal bovine se-rum, 100 I.U. mL⁻¹ penicillin/streptomycin (Thermo Fisher Scientific). MOC2-luc cells were cultured in the mixture of IMDM medium and Ham's Nutrient Mixture F12 Media (at a ratio of 2:1, GE Health Healthcare Life Sciences) supplemented with 5% fetal bovine serum, 100 I.U. mL⁻¹ penicillin/streptomycin, 5 ng mL⁻¹ insulin (MilliporeSigma), 40 ng mL⁻¹ hydrocortisone (MilliporeSigma), and 3.5 ng mL⁻¹ human recombinant EGF (MilliporeSigma). All cells were cultured in a humidified incubator at 37° C. in an atmosphere of 95% air and 5% CO₂.

In Vitro NIR-PIT with Reducing Agents

A431 GFP-luc or MDAMB468 GFP-luc cells (4×10⁵) were seeded into 12-well plates, and incubated for 24 hours, and then exposed to media containing panitumumab-IR700 (10 μg mL⁻¹) for 1 hour at 37° C. After aspirating media with APC, phenol-red-free media containing various concentration of L-NaAA, L-cysteine, and/or NaN₃ were added in each well. NIR light (690 nm, 150 mW cm⁻², 1 or 2 J cm⁻² for A431 GFP-luc, 20 J cm⁻² for MDAMB468 GFP-luc) was irradiated to cancer cells. For flow cytometric analyses, the cells were collected with trypsin one hour after NIR light exposure. Cells were then stained with propidium iodide (PI, 1 μg mL⁻¹) at RT for 5 minutes. The fluorescence of the cells was then analyzed with a flow cytometer (FACSCalibur, BD Biosciences, San Jose, CA, USA) and FlowJo software (BD Biosciences).

### Animals and Tumor Models

All in vivo procedures were conducted in compliance with the Guide for the Care and Use of Laboratory Animal Resources (1996), US National Research Council. Six- to eight-week-old female C57BL/6 mice and homozygote athymic nude mice were from The Jackson Laboratory (Bar Harbor, ME, USA) and Charles River Laboratories (Wilmington, MA, USA), respectively. A431 GFP-luc (1×10⁶), MC38-luc (1×10⁶), LL/2-luc (0.5×10⁶), or MOC2-luc (1×10⁶) cells were inoculated into the right side of the dorsum. The hair overlying the tumor site was removed before NIR-PIT and imaging for C57BL/6 mice. Body weight was measured from the day before to 4-6 days after NIR light exposure. The mice were euthanized with CO₂ when all experiments involving BLI were finished.

### In Vivo NIR-PIT

C57BL/6 tumor-bearing mice were randomized into three groups as follows: i) no treatment (control), ii) intravenous administration of APCs followed by NIR light exposure (NIR-PIT) without L-NaAA, iii) NIR-PIT with L-NaAA. The APC was injected 6 days after inoculation of cancer cells into C57BL/6 mice. Similarly, tumor-bearing mice of athymic mice were randomized into two groups as follows: i) NIR-PIT without L-NaAA, ii) NIR-PIT with L-NaAA. The dose of APC was 100 μg for CD44-IR700, and 50 μg for CTLA4-IR700, respectively. NIR light (690 nm, 150 mW cm-2, 50 J cm-2) was exposed on the next day. L-NaAA (80 mg mouse-1) or PBS was injected intraperitoneally 15 minutes before NIR light exposure. Upon NIR light exposure, the normal tissue adjacent to the tumor was covered with aluminum foil to ensure that the NIR light exposure is limited in tumor site. Dorsal fluorescence images of IR700 were obtained with the 700 nm fluorescence channel of the Pearl Imager (LI-COR Biosciences). The images were taken pre- and post-NIR-PIT. ROIs were placed on the tumor. The dorsal edema formation of treated mouse 1 day after NIR light irradiation was imaged by a camera. Acute treatment efficacy was evaluated with BLI analysis, in which, D-luciferin (3 mg mouse-1; Gold Biotechnology) was intraperitoneally injected into mice. Luciferase activity was analyzed with a BLI system (Biospace Lab) using relative light units. ROIs were placed over the entire tumor. The counts per minute of relative light units were calculated using M3 Vision Software (Biospace Lab) and converted to the percentage based on those before NIR-PIT using the following formula: [(relative light units after treatment)/(relative light units before treatment)×100(%)]. Bioluminescence im-ages were continually recorded until the onset of depilation-induced skin pigmentation precluded accurate measurement.

### Magnetic Resonance Imaging (MRI)

Under pentobarbital anesthesia, MRI was performed on a 3-T scanner using an in-house 10-inch circle shaped mouse receiver coil array (Elition 3T; Philips Medical Systems, Best, Nether-lands) 1 day after NIR light exposure. Scout images were obtained to accurately locate the tumor. All mice underwent T2-weighted fat-saturation imaging (T2WI fat-sat) and short-TI inversion recovery imaging (STIR). All images were obtained in the coronal plane and T2WI fat-sat images were also obtained in the axial plane. All images were analyzed using Image J software, and 3D imaging were recon-structed. The high signal intensity area derived from each image in T2WI fat-sat and STIR was calculated by Image J.

### Flow Cytometric Analysis

To confirm the depression of CTLA4 expressing cells, the tumors of MC38-luc were harvested 3 hours after NIR light expo-sure. Single-cell suspensions from tumor samples were pre-pared using following protocol. Whole tumors were incubated in the RPMI 1640 medium (Thermo Fisher Scientific) containing collagenase type IV (1 mg mL⁻¹; Thermo Fisher Scientific) and DNase I (20 μg mL⁻¹; Millipore Sigma, Burlington, MA) in 37° C. for 30 minutes, then gently cut with scissors and mashed with the back of the plunger of 3 mL syringe. The tissues were passed through a 70 μm cell strainer (Corning, Corning, NY). A total of 3.0×10⁶ cells was stained and data for 5.0×10⁵ cells were collected for each tumor. The cells were stained with anti-CTLA4 antibody (Clone; UC10-4B9) from Thermo Fisher Scientific. To distinguish live cells from dead cells, cells were also stained with LIVE/DEAD Fixable Dead Cell Stain (Thermo Fisher Scientific). The fluorescence of the cells was then analyzed with the flow cytometer (FACSLyric, BD Biosciences) and FlowJo software (FlowJo LLC). Dead cells were removed from analysis based on FSC, SSC, and staining with LIVE/DEAD Fixable Dead Cell Stain.

### Statistical Analysis

Data are expressed as means±SEM. Statistical analysis was per-formed with GraphPad Prism (GraphPad Software, LaJolla, CA, USA). Sample size (n) for each experiment is described in each figure legend. For one-time measurement, a two-tailed unpaired t test (two groups) or a one-way analysis of variance (ANOVA) followed by Tukey's test or Dunnett's test (three or more groups) was used. For comparison of luciferase activity, biodistribution, and body weights, a repeated measures two-way ANOVA followed by Tukey's test was used. p-value of less than 0.05 was considered significant.

### Example 5

### L-NaAA Accelerates Release of Axial Ligands with NIR Irradiation Regardless of the Presence of Sodium Azide (NaN₃)

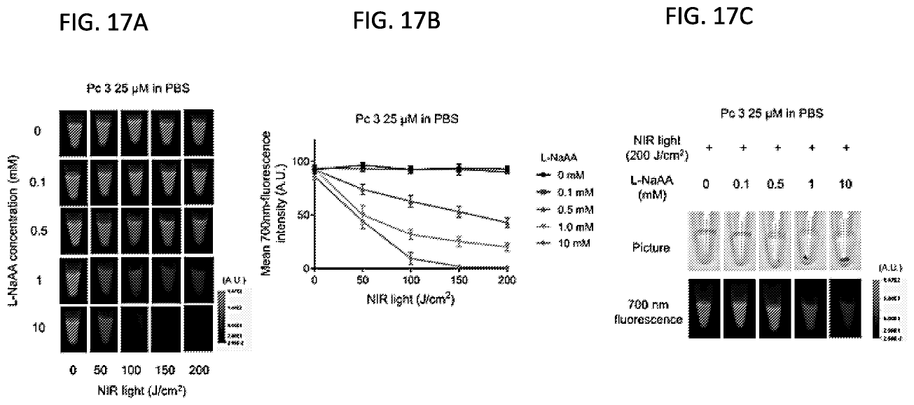

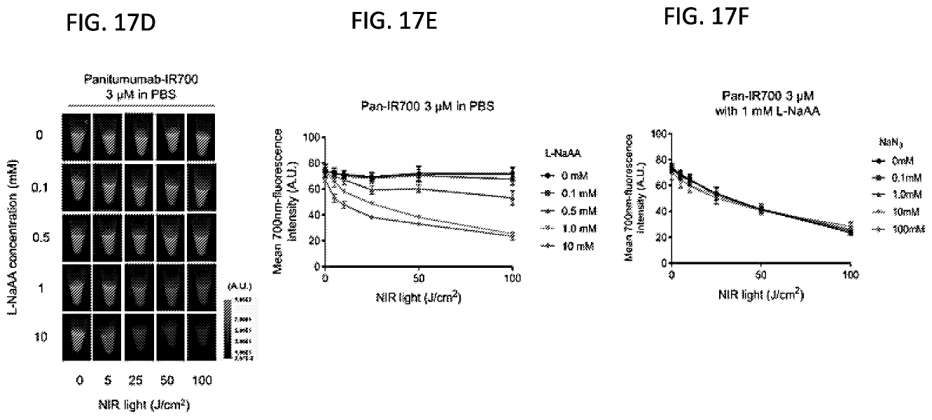

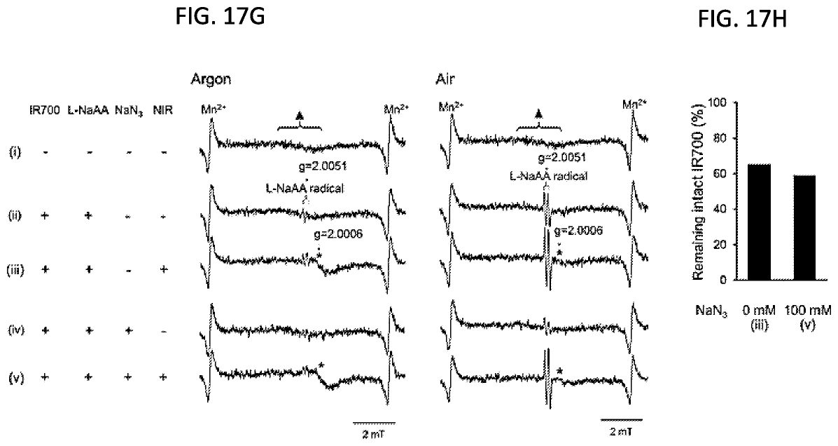

The compound Pc 3, the phthalocyanine moiety of IR700, was synthesized according to FIG. 16. The photo-induced ligand release reaction of Pc 3 after NIR light exposure was accelerated in the presence of L-NaAA, an electron donor, in a dose-dependent manner. The 700 nm fluorescence of Pc 3 was decreased in a dose-dependent manner of NIR light and L-NaAA (FIGS. 17A, 17B). The loss of fluorescence was associated with precipitation of Pc 3 in a L-NaAA dose-dependent manner because Pc 3 dramatically changed its solubility after light expo-sure (FIG. 17C). Fluorescence emission of IR700 conjugated with panitumumab (panitumumab-IR700) was decreased in a dose-dependent manner of NIR light and L-NaAA (FIGS. 17D, 17E). The presence of NaN3, L-NaAA did not inhibit the decrease of panitumumab-IR700 fluorescence after NIR light irradiation (FIG. 17F).

To identify intermediate products of IR700 and L-NaAA radicals, electron spin resonance (ESR) spectroscopy was performed. A broad signal was observed in the central field (FIG. 17G (i)), however, this signal was also observed in empty quartz ESR tubes indicating this signal was derived from the quartz ESR tube itself. Thus, this signal could be subtracted and did not interfere with the ESR measurements. Under argon-saturated conditions (FIG. 17G, left), a small but clear doublet signal with g-value of 2.0051 and splitting width of 0.18 mT was observed just by mixing IR700 with L-NaAA for several minutes (FIG. 17G (ii)). The ESR parameters of this signal are almost the same as those reported previously for the L-NaAA radical, which is produced during oxidation of L-NaAA. This observation indicates that L-NaAA radicals are produced by redox interaction between IR700 and L-NaAA without NIR light irradiation. When the sample containing 10 mM L-NaAA and 0.5 mM IR700 was irradiated with NIR light, a small signal with broad line width of 0.87 mT and g-value of 2.0006 appeared as indicated by an as-terisk in the left spectrum of FIG. 17G (iii). Since this signal was not observed in the absence of an electron donor such as L-NaAA and the intensity of the signal in air, where electrophilic molecule oxygen is present, was much smaller than that in the argon saturated condition, this ESR signal was assigned to the IR700 anion radical produced by the electron transfer from L-NaAA to the NIR light-induced triplet state IR700. In this procedure, the scanning of the magnetic field and sample irradiation started at the same time and continued for 8 min/10 mT. Therefore, the central region where the signal due to IR700 anion radical was detected occurred about 4 min into the scan. This signal derived from IR700 anion radical was observed for at least 16 minutes after NIR light irradiation. This signal was derived from the IR700 anion radical, indicating that NIR light irradiation produces IR700 anion radicals in the presence of L-NaAA. In the left spectra of FIG. 17G (iv) and (v), the effect of NaN3 on the formation of L-NaAA radicals and NIR light-induced IR700 anion radicals was examined. The results showed that addition of 100 mM NaN₃ attenuated the formation of L-NaAA radicals, but did not affect the formation of IR700 anion radicals produced by NIR light irradiation.

A similar experiment was carried out in the presence of oxygen. As shown in the right spectra of FIG. 17G (ii) and (iii), the reaction of IR700 with L-NaAA in air showed a stronger radical signal de-rived from L-NaAA radicals than under argon saturated conditions, and the L-NaAA radicals were further enhanced by NIR light irradiation. Moreover, it was observed that NIR light irradiation produced IR700 anion radicals in air, but it was greatly reduced compared to the argon-saturated condition. The effect of NaN3 on L-NaAA radicals and IR700 anion radicals is shown in the right spectra of FIG. 17G (iv) and (v). The addition of 100 mM NaN3 to the sample decreased the ESR signal intensity derived from L-NaAA radical but did appear to affect the formation of IR700 anion radical produced by NIR irradiation. The axial ligand cleavage was observed in L-NaAA added condition, and it was not enhanced by NaN₃ (FIG. 17H).

### Example 6

### NaN3 Inhibited Ligand Release of IR700 when L-Cysteine was Added as an Electron Donor

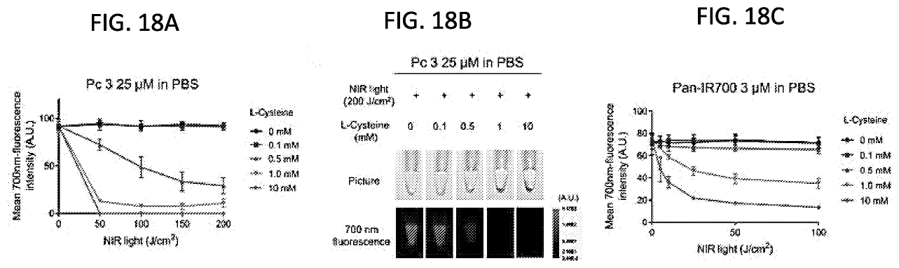

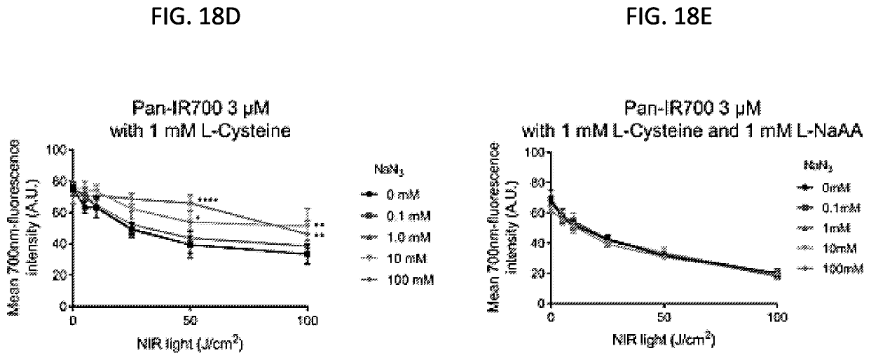

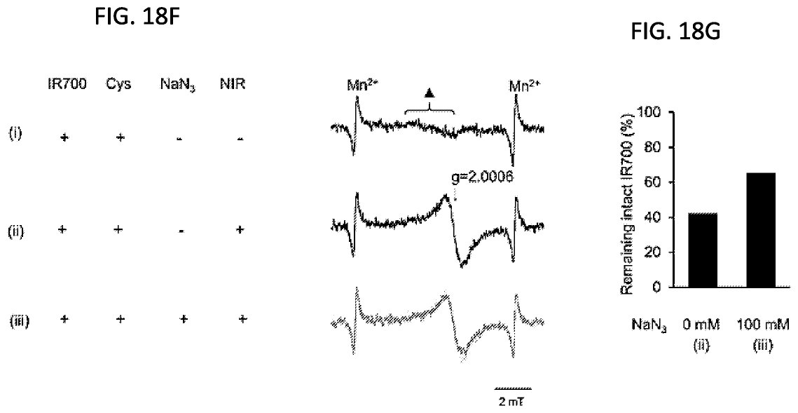

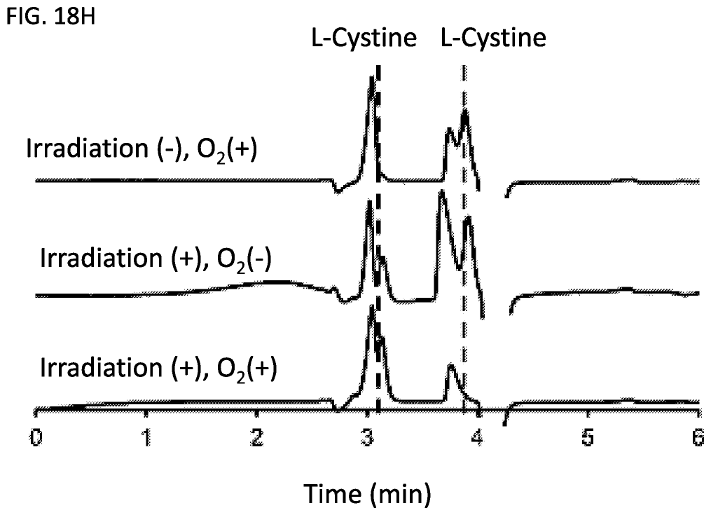

The photo-induced ligand release reaction of Pc 3 and panitumumab-IR700 was evaluated under conditions of added L-cysteine, which is an alternative electron donor to L-NaAA. A similar phenomenon was observed, but it occurred at lower NIR light irradiation levels than when L-NaAA was used (FIG. 18A-18C). With concentrations of NaN₃ of 10 mM or higher combined with 1 mM L-cysteine, loss of panitumumab-IR700 fluorescence seen with L-cysteine alone was compromised (FIG. 18D). However, the addition of 1 mM L-NaAA to 1 mM L-cysteine counteracted the effects of NaN₃ (FIG. 18E).

The control ESR spectrum of 0.5 mM IR700 and 10 mM L-cysteine without irradiation is shown in FIG. 18F (i). When the sample of 0.5 mM IR700 and 10 mM L-cysteine was dissolved in 100 mM phosphate buffer solution (pH 7.0) and irradiated with NIR light under argon saturated conditions, a symmetrical ESR spectrum with a line width of 0.87 mT and a g-value of 2.0006, which was assigned to IR700 anion radical, was observed as shown in FIG. 18F (ii). Furthermore, to evaluate the effect of NaN₃ on NIR light-induced IR700 anion radicals, similar experiments were carried out in the presence of 100 mM NaN₃ in the sample. The ESR spectrum in the presence of NaN₃ (red color) was superimposed on the spectrum in the absence of NaN₃ (gray color; same spectrum as FIG. 18F (ii)) as shown in FIG. 18F (iii). These results showed that NaN₃ decreased the signal of IR700 anion radical. When NaN₃ was added, ligand cleavage decreased (FIG. 18G). This result is consistent with the results of ESR, in which the formation of radical anions was inhibited.

To determine whether L-cystine is produced from L-cysteine, the solution of IR700 was irradiated in the presence of L-cysteine, and analyzed by high performance liquid chromatography (HPLC). When IR700 was irradiated under hypoxic conditions, the peaks of L-cysteine and L-cystine were observed, although there was no peak of L-cystine without NIR light irradiation (FIG. 18H). On the other hand, upon NIR light irradiation in the presence of oxygen, the peak of L-cysteine was greatly reduced while the peak of L-cystine was larger than under hypoxic conditions. These results indicated that L-cysteine acted as a reductant to IR700 in the excited state and was converted to L-cystine. In addition, L-cysteine was consumed more efficiently in the presence of oxygen than in the absence of oxygen. This may be because oxygen quenches IR700 in the triplet excited state or the anion radical in the ground state, resulting in a more efficient cycle of excitation and reduction.

### Example 7

### L-NaAA Counteracts the Suppression of NIR-PIT Cytotoxicity Caused by NaN₃

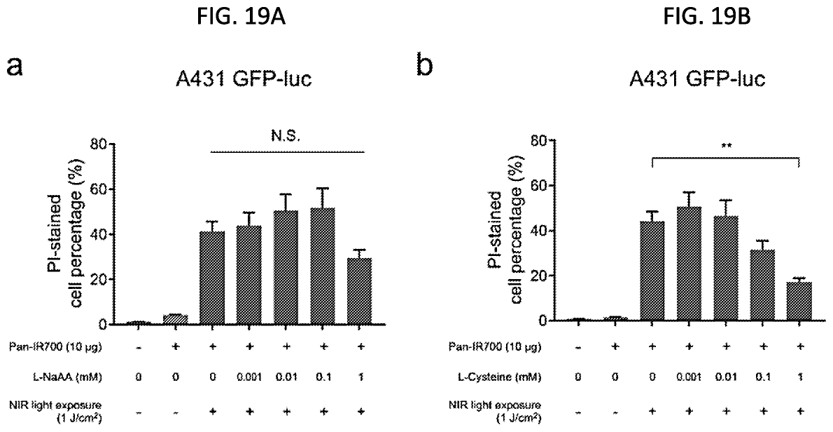

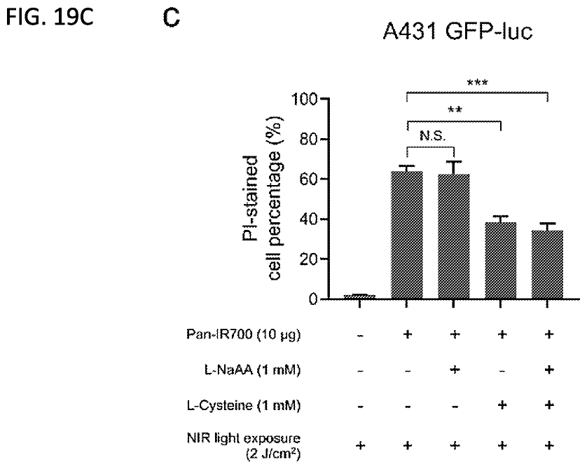

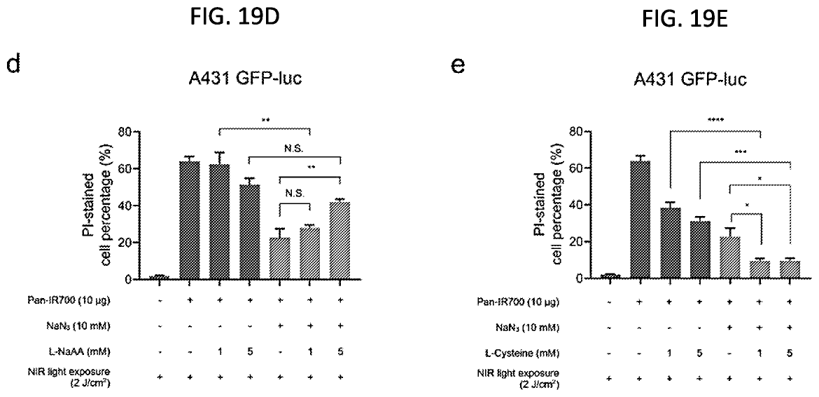

To investigate the effect of reducing agents on the effectiveness of NIR-PIT, the cytotoxic effects of NIR-PIT targeting cancer cells were quantitatively assessed by cell viability assays. Under low or pharmacological concentrations, L-NaAA enhanced the cytotoxic damage of panitumumab-IR700 NIR-PIT (Pan-PIT). Under supraphysiologic concentrations of L-NaAA (e.g., over 1 mM), cytotoxic damage by Pan-PIT was slightly inhibited. In contrast, L-cysteine slightly enhanced the cytotoxic damage of Pan-PIT but only at very low concentrations like 0.001 mM, while it compromised the cytotoxicity at concentrations of 0.1 mM or higher (FIGS. 19A-19B). When both L-cysteine and L-NaAA were present in equal concentration, L-cysteine tended to counteract the effects of L-NaAA because of the higher redox potential of L-cysteine compared to L-NaAA (FIG. 19A).

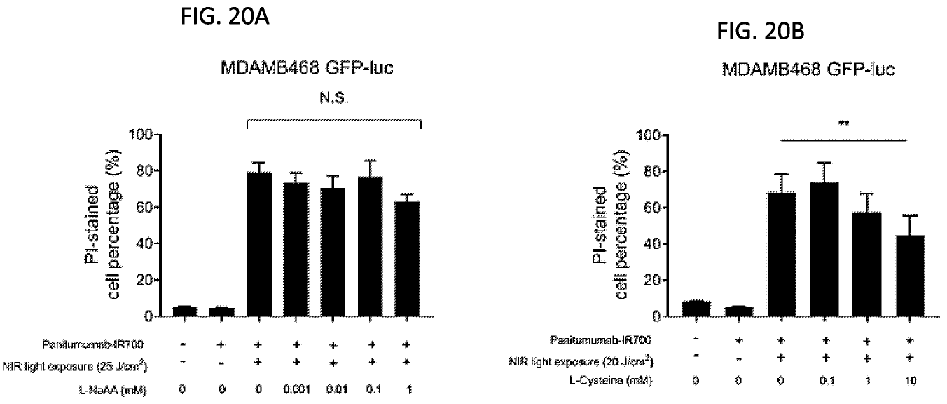

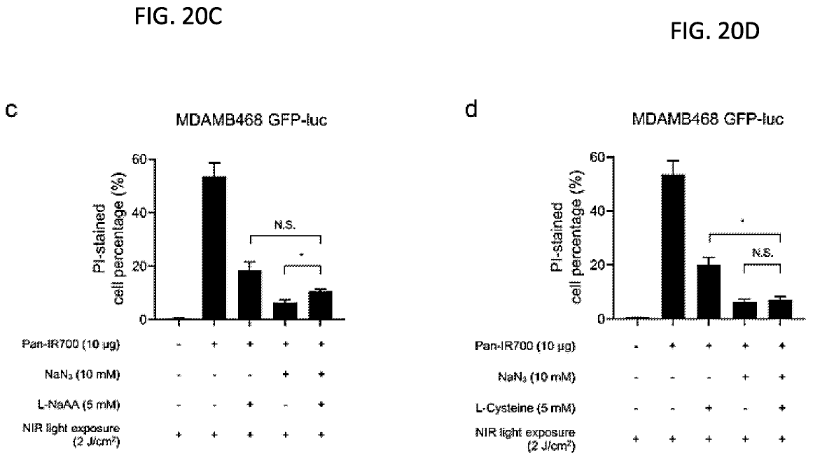

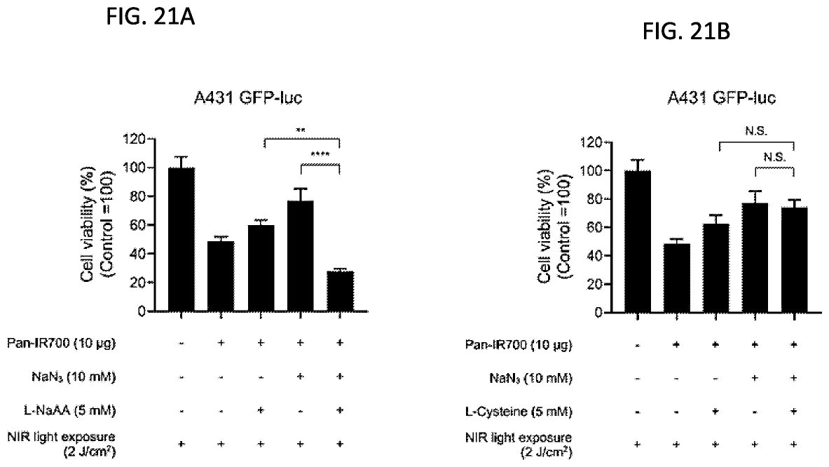

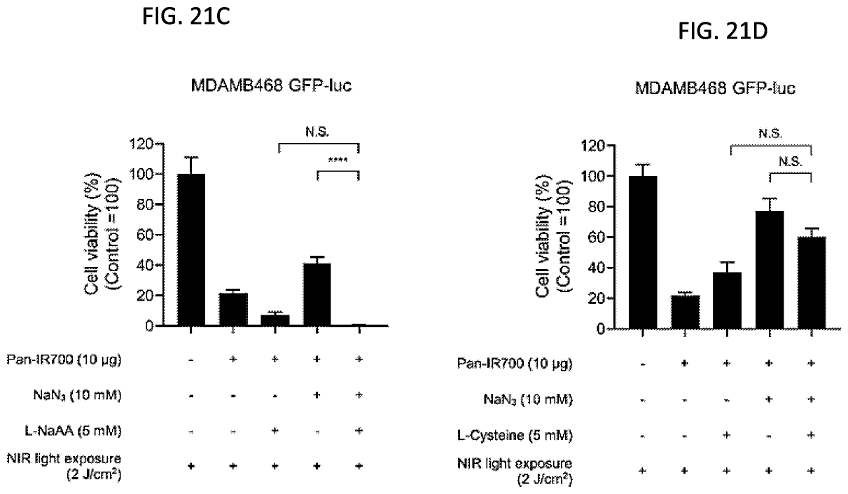

Next, the impact of each reducing agent on cytotoxicity of Pan-PIT in the presence of NaN₃ was evaluated. L-NaAA overcame the reduced cytotoxicity caused by NaN3, but L-cysteine could not reverse the effects of NaN3 (FIGS. 19D, 19E). In another cell line, MDAMB468, L-NaAA also restored cytotoxicity of Pan-PIT suppressed by NaN3 (FIGS. 20A-20D). A similar tendency was observed by bioluminescence imaging (BLI) (FIGS. 21A-21D). These results demonstrate that the suppression of NIR-PIT cytotoxicity by NaN₃ was overcome by L-NaAA, but not by L-cysteine, despite of its higher redox potential, probably because added L-cysteine formed disulfide bonds with the sulfhydryl groups of L-cysteine which compromised its ability to accelerate the photo-induced ligand release reaction.

### Example 8

### L-NaAA Accelerates Ligand Release of IR700 after NIR Light Exposure

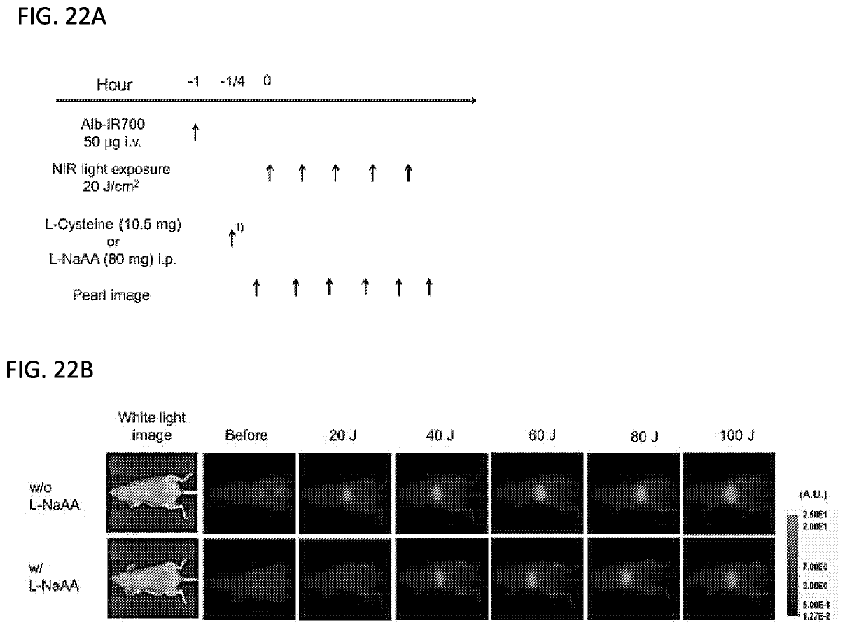

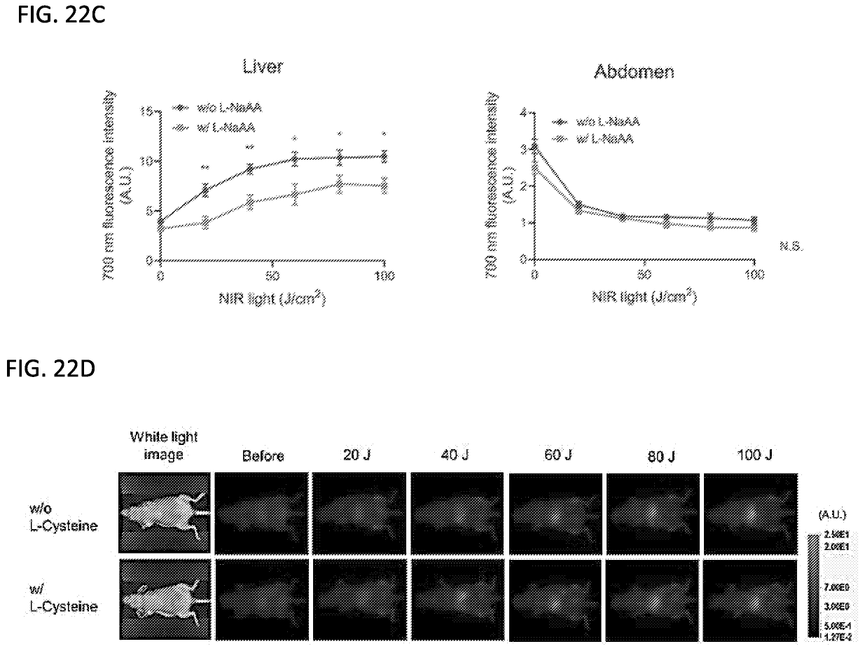

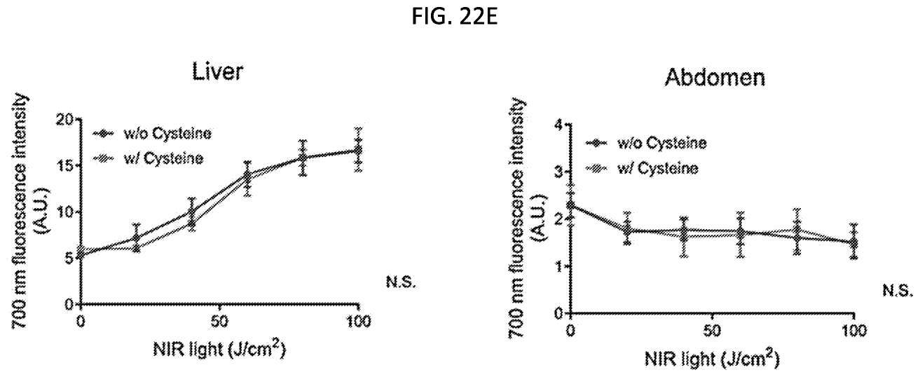

To evaluate whether L-NaAA or L-cysteine better facilitates ligand release from IR700 in vivo, albumin-IR700 (alb-IR700) was used and loss of fluorescence measured (FIG. 22A). Conjugated alb-IR700 (50 μg) was injected via lateral tail vein in non-tumor-bearing athymic nude mice 2 hours before NIR light exposure. L-NaAA (80 mg), L-cysteine (10.5 mg), or PBS was injected via tail vein 2 minutes before irradiating the mouse with NIR light. The abdomen was exposed to NIR light (690 nm, 150 mW cm⁻², 20 J cm⁻²). Ventral fluorescence was measured before and after NIR light exposure using the Pearl Imager (LI-COR Biosciences). The cycle of NIR light irradiation and fluorescence imaging was repeated 5 times. ROIs were placed on the liver and the irradiated site. Average fluorescence intensity of each ROI was calculated.

After exposing alb-IR700 to NIR light, the signal of 700 nm fluorescence decreased at the irradiated site, whereas, the signal within liver increased in a NIR light dose-dependent manner (FIG. 22B, 22D). Under L-NaAA administration, fluorescence was significantly decreased compared to the condition without L-NaAA, however, there were no obvious changes in fluorescence with L-cysteine (FIG. 22C, 22E). Decreased 700 nm fluorescence in the liver means that superior ligand release occurred with alb-IR700 after NIR light exposure, because ligand release results in loss of fluorescence. Thus, L-NaAA accelerated ligand release of IR700 conjugated to albumin in vivo, but L-cysteine did not.

### Example 9

### L-NaAA Did not Affect the Efficacy of Cancer Cell-Targeted NIR-PIT

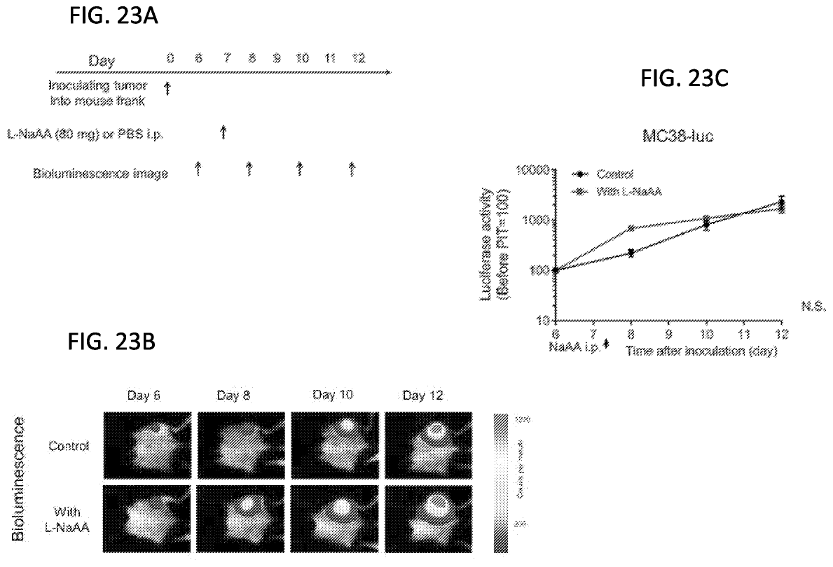

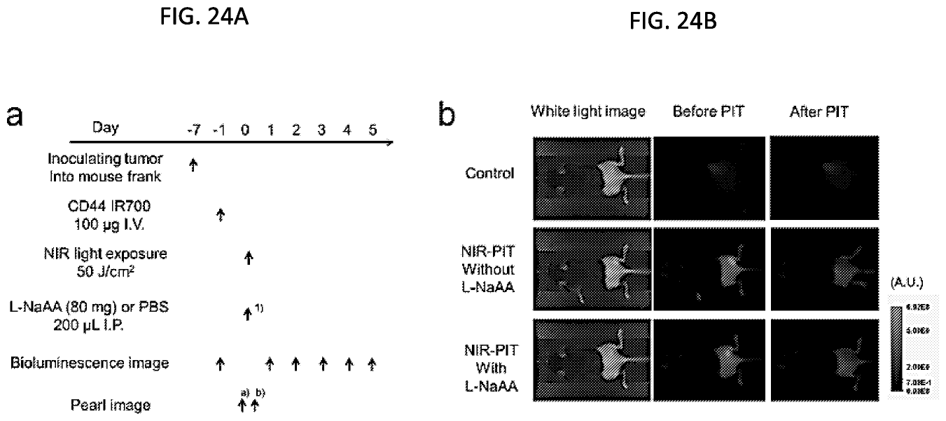

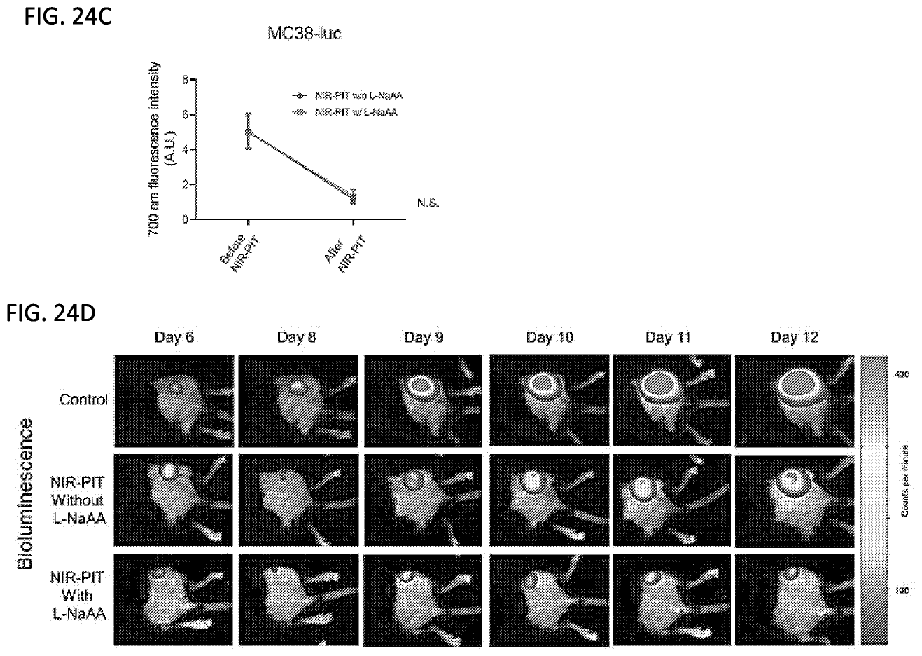

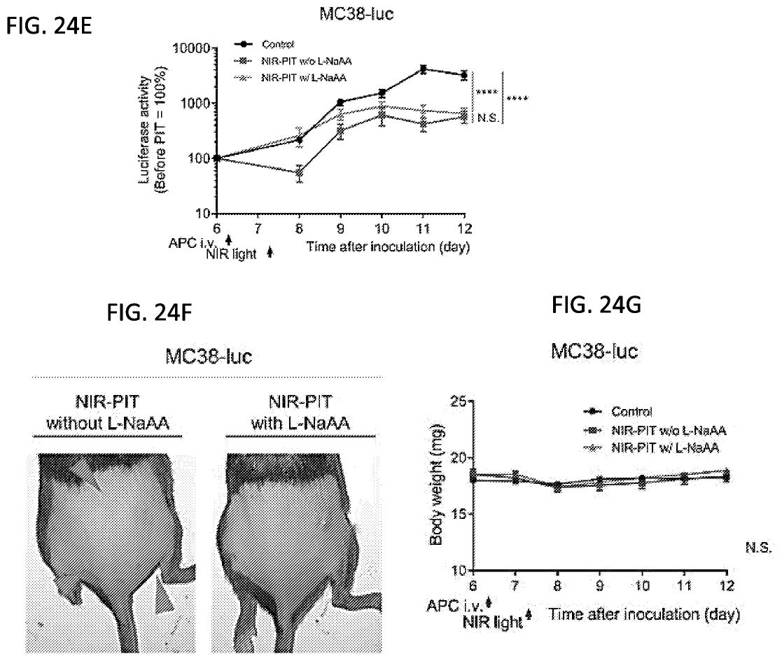

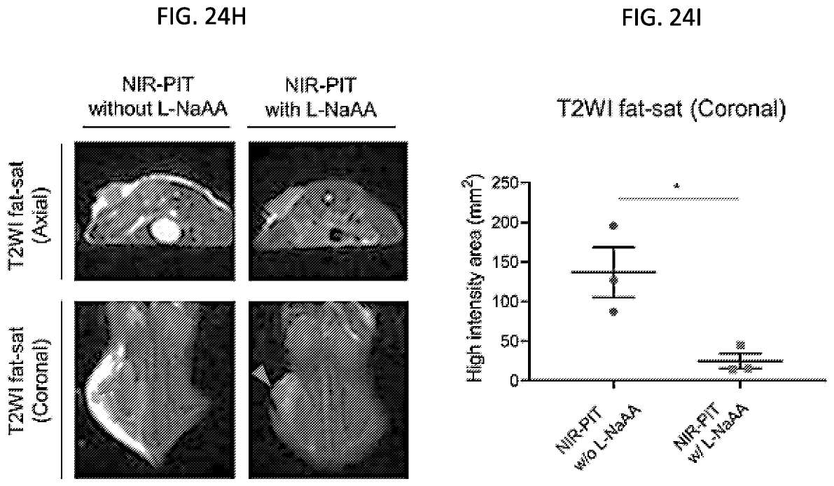

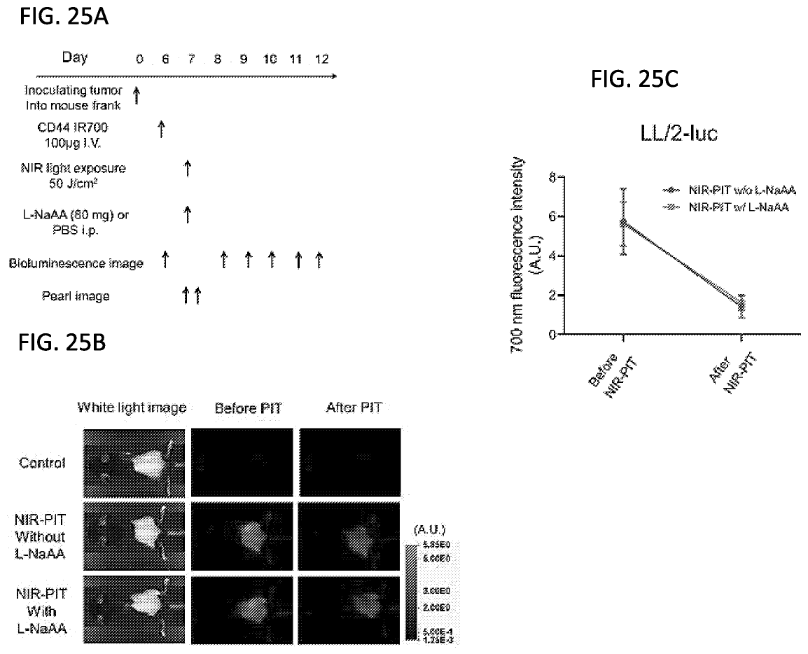

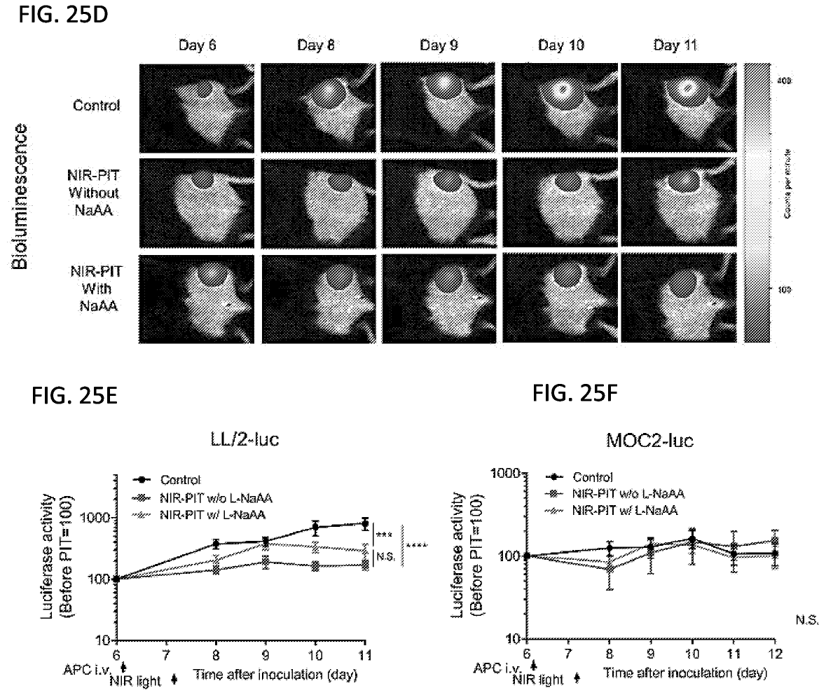

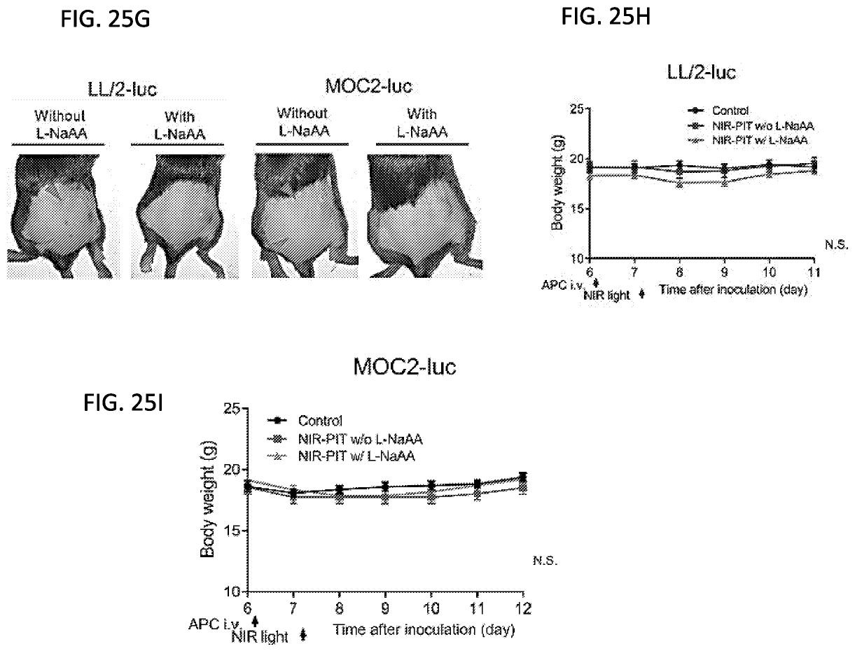

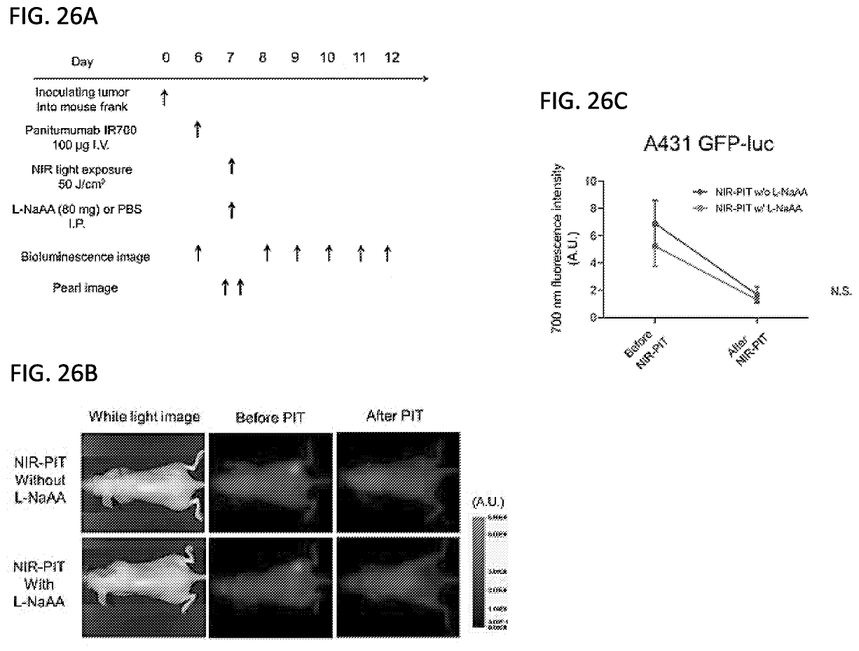

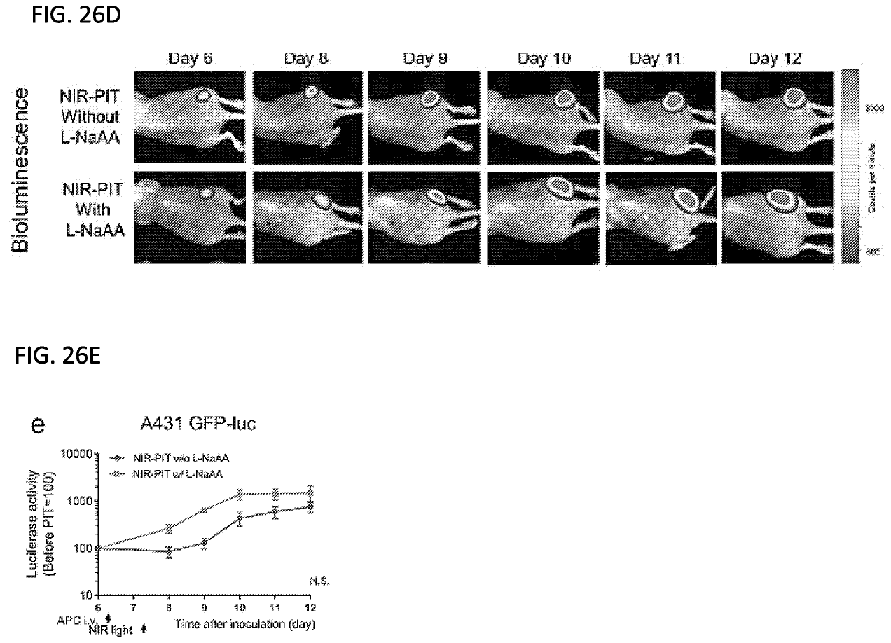

It was determined if L-NaAA affected the antitumor effects of cancer cell-targeted NIR-PIT utilizing a CD44-IR700 in vivo allograft tumor model. First, L-NaAA was confirmed not to affect tumor growth. The treatment and imaging regimen is shown (FIG. 23A). The accumulation of CD44-IR700 within MC38-luc tumors was confirmed by fluorescence imaging, and this decreased after NIR light exposure regardless of whether L-NaAA was present (FIG. 23B, 23C). The therapeutic efficacy of CD44-targeted NIR-PIT was evaluated with BLI as a readout (FIG. 24D). Regardless of the presence of L-NaAA, both NIR-PIT groups showed significantly lower intensity on BLI compared to the control group indicating cancer cell death and there was no significant difference between NIR-PIT with L-NaAA and without L-NaAA (FIG. 24E). In the NIR-PIT group without L-NaAA on day 1, severe edema formation was observed in the skin over the tumor. This likely attenuated much of the bioluminescence signal. The MC38-luc, LL/2-luc, and MOC2-luc tumor models all showed that the antitumor effects of CD44-targeted NIR-PIT were not changed with L-NaAA (FIGS. 25A-25I). In athymic mice, L-NaAA also did not affect the efficacy of pan-PIT (FIGS. 26A-26E).

### Example 10

### L-NaAA Suppressed Edema Formation after Treatment with NIR-PIT

Edema formation was assessed after CD44-targeted NIR-PIT with and without L-NaAA. In MC38-luc, LL/2-luc, and MOC2-luc tumor-bearing mice, edema was observed on the dorsal surface around the subcutaneous tumor 1 day after NIR-PIT in mice without L-NaAA (FIGS. 24F and 25G). However, no edema was detected in mice treated by NIR-PIT with L-NaAA injection. No significant difference in body weight were observed between the two groups (FIG. 24G). In other allograft models, no significant difference in body weight was detected after CD44-targeted NIR-PIT with L-NaAA (FIGS. 25H, 25I).

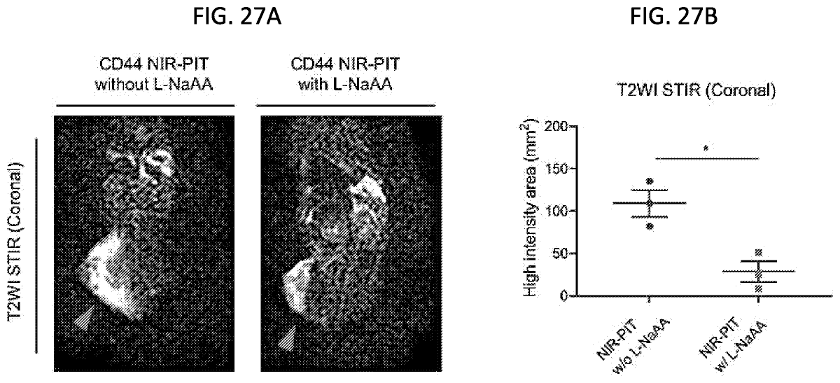

To quantify the degree of edema, magnetic resonance imaging (MRI) was performed 1 day after CD44-targeted NIR-PIT. In the non L-NaAA group, T2-weighted and fat saturation images (T2WI fat-sat) showed extensive edema depicted as high signal intensity areas extending from the peripheral tumor site to the lower edge of the thorax and to the proximal side of the tail in NIR-PIT. In contrast, in the L-NaAA group, minimal edema was observed just around the treated tumors (FIG. 25H). The area of high signal intensity corresponding to post treatment edema on the T2WI fat-sat images was quantified and showed that the high signal area was significantly larger in the group treated without L-NaAA vs. the L-NaAA group (FIG. 25I). This was also confirmed on the short-TI inversion recovery images (STIR) (FIGS. 27A-27B).

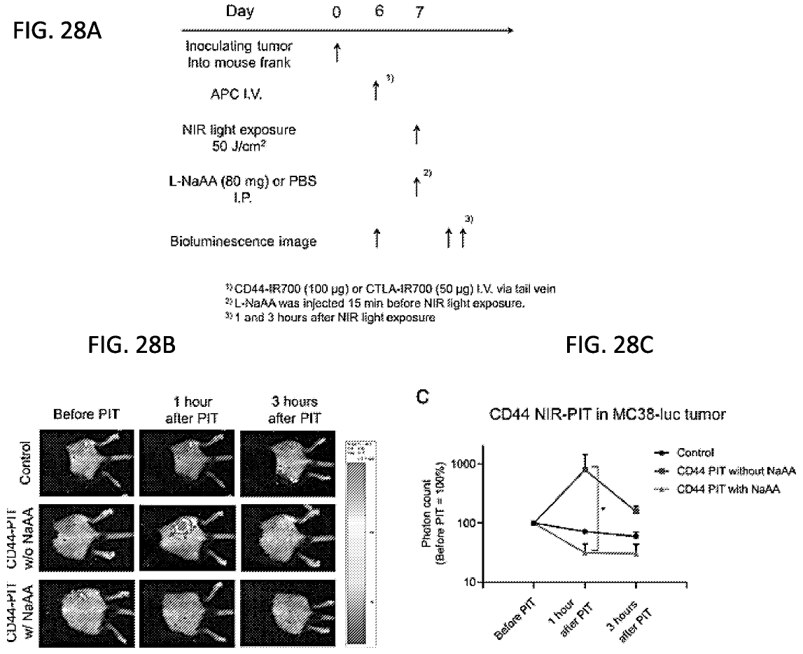

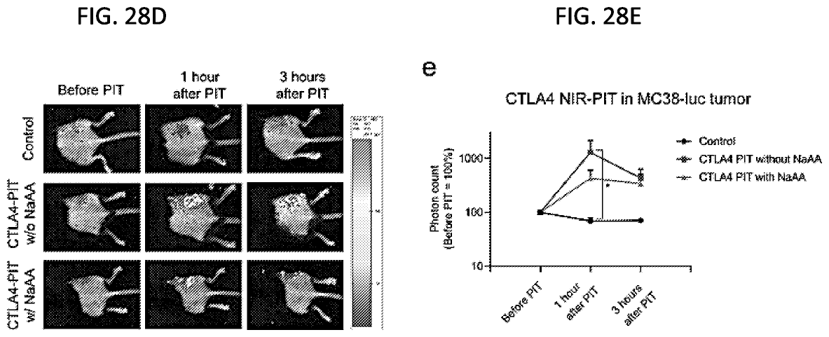

To investigate the cause of edema formation, ROS was evaluated with chemiluminescence imaging (CLI) using L-012 in MC38-luc tumor-bearing mice before and after NIR-PIT (FIGS. 28A-28C). The CD44-targeted NIR-PIT with L-NaAA showed significantly lower intensity (lower ROS) on CLI compared with NIR-PIT without L-NaAA. These data indicated that L-NaAA inhibited edema formation induced by ROS after NIR-PIT.

### Example 11

### L-NaAA Suppressed Edema Formation after NIR-PIT Targeting Immuno-Suppressive Cells without Interfering with Efficacy

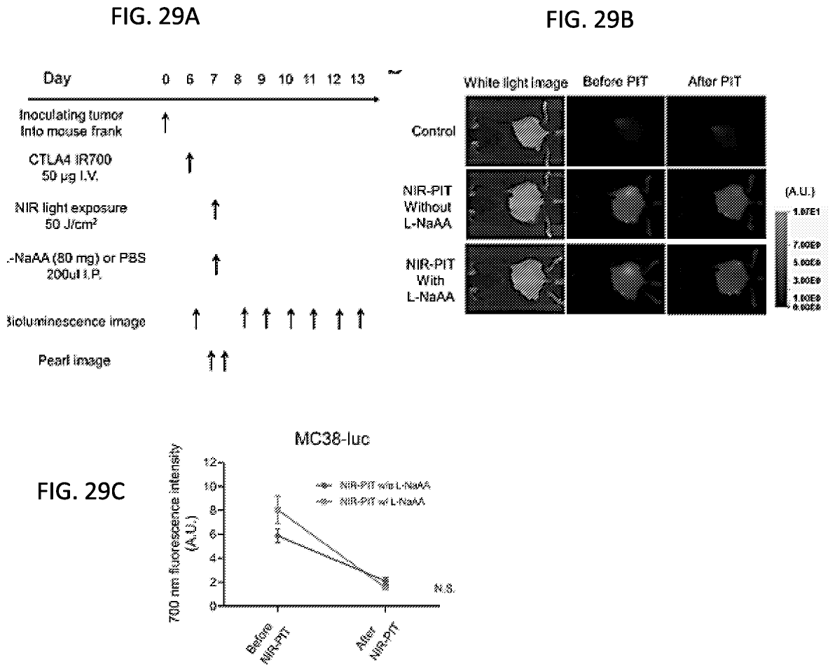

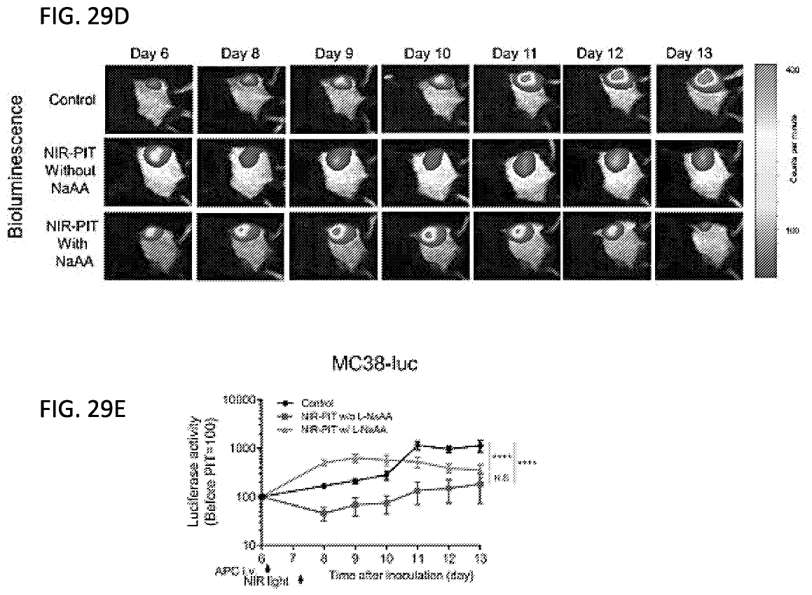

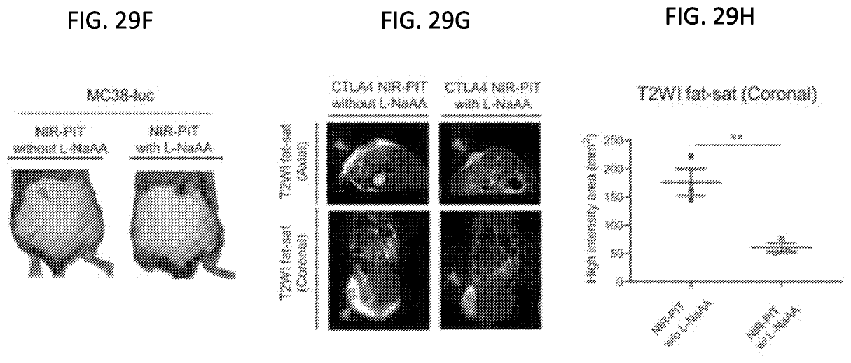

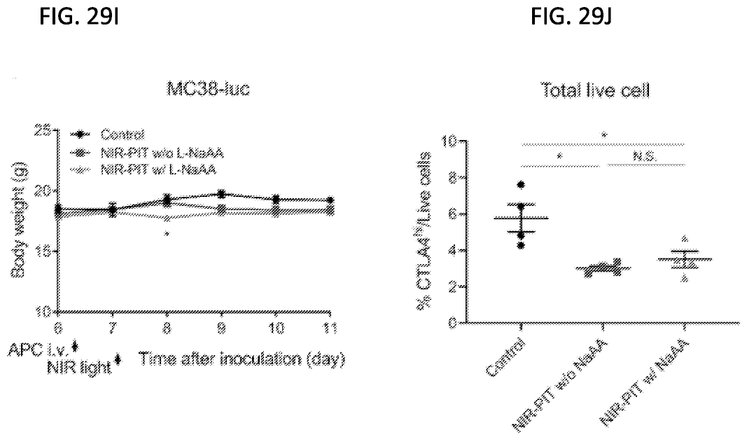

The efficacy of CTLA4-targeted NIR-PIT with and without L-NaAA was evaluated in MC38-luc tumors. In both groups, CTLA4-IR700 fluorescence was observed one day post-injection prior to NIR-PIT but decreased after NIR light exposure (FIGS. 29A-29C). The NIR-PIT group without L-NaAA had lower BLI tumor signal than the NIR-PIT group with L-NaAA at day 1 to day 3 after NIR light exposure (FIG. 29D). However, by day 6 post NIR-PIT, no difference was observed between the two groups, and both NIR-PIT groups showed significantly lower BLI intensity compared to the control group (FIG. 29E). Since CTLA4-targeted NIR-PIT showed stronger edema formation than CD44-targeted NIR-PIT, a lower intensity on BLI was shown in the NIR-PIT without L-NaAA group due to the skin thickening. Edema formation in mice treated by NIR-PIT with L-NaAA was also suppressed both macroscopically and by MRI (FIGS. 29F-29H). The day after NIR-PIT, a significant lower weight was observed in mice in the NIR-PIT with L-NaAA group (FIG. 291) perhaps due to the comparative lack of edema.

In CTLA4-targeted NIR-PIT, higher ROS was also detected in the NIR-PIT without L-NaAA group (FIGS. 28D, 28E). To confirm whether selective cytotoxicity of CTLA4-targeted NIR-PIT was preserved in the NIR-PIT with L-NaAA group, selective depletion of CTLA4 expressing cells was assessed in MC38-luc tumors three hours after CTLA4-targeted NIR-PIT (FIG. 29J). CTLA4hi cells were selectively depleted in both NIR-PIT with and without L-NaAA groups. Thus, L-NaAA had no effect on the efficacy of immune-suppressive cell-targeted NIR-PIT but it did suppress edema formation by ROS.

Selective cytotoxicity induced by NIR-PIT is caused by the photoinduced ligand release reaction which occurs in hypoxic electron donor rich conditions. The ligand release causes dramatic changes in solubility of the APC-antigen complex causing damage to the cell membrane and cell death. However, under normoxic or hyperoxic conditions NIR light induces ROS production in the APC that which likely contributes to non-selective cytotoxicity and local edema following NIR-PIT. An-other possible cause of local edema formation may be lymphatic obstruction associated with direct lymphatic damage due to NIR-PIT. However, no obvious lymphatic occlusion or morpho-logical changes by NIR-PIT were observed. Thus, ROS increases vascular permeability resulting in edema formation. L-NaAA is an electron donor that should facilitate NIR-PIT ligand release reaction, yet quenches most of ROS thereby reducing edema as shown in FIGS. 24H and 29G on MRI. Since ROS quenching could interfere with cancer cell cytotoxicity, it might affect the therapeutic effects of NIR-PIT. However, NIR-PIT with L-NaAA injection still showed acceptable cytotoxicity but without edema formation. Therefore, L-NaAA administration on NIR-PIT provides an effective therapy that is safer, for example when edema could affect the airways or mediastinum.

Under hypoxic conditions, L-NaAA radical production was minimal but under normoxic conditions, the amount of L-NaAA radical production was extremely large (FIG. 17G (ii) and (iii)), indicating that oxygen molecules are involved in the production of the L-NaAA radical. It is possible that the IR700 anion radical donates one electron to oxygen to produce superoxide anion radical (O₂—·) in the presence of oxygen, or the triplet state IR700 generated by photo excitation gives energy to triplet oxygen (³O2) to produce singlet oxygen (¹O2). It has been reported that L-NaAA reacts with O₂—· and ¹O2 to form L-NaAA radical and hydrogen peroxide. Therefore, it is possible that in the presence of oxygen, L-NaAA may react with these ROS, resulting in the generation of many L-NaAA radicals. In this case, large doses of L-NaAA could consume generated ROS, reducing the side-effects of NIR-PIT in clinical practice.

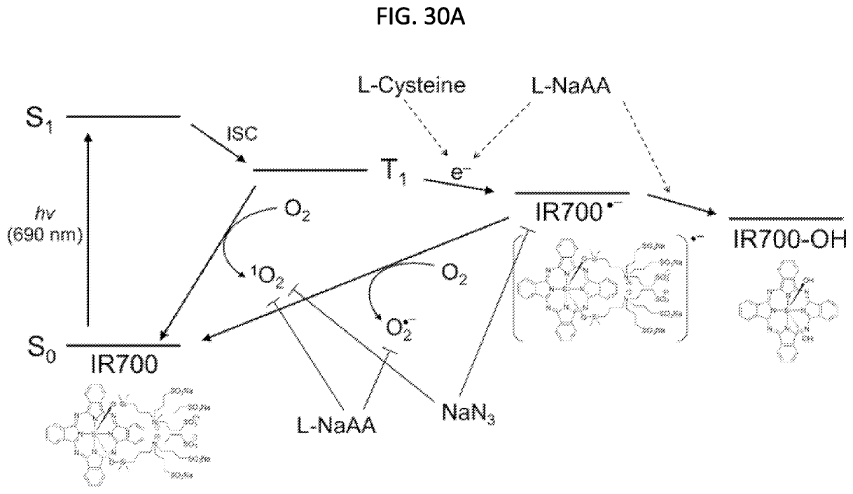

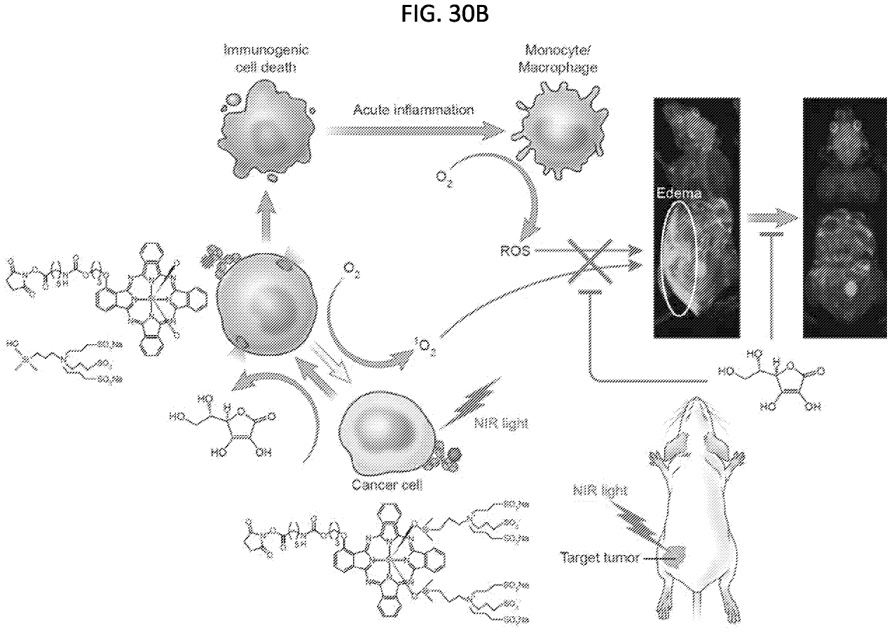

Although IR700 anion radical was clearly observed when L-cysteine was used as the reducing agent (FIG. 18F (ii)), the signal was very low when L-NaAA was used as the reducing agent (FIG. 17G (iii)). This may be due to the difference in the properties of L-cysteine and L-NaAA as reducing agents. L-Cysteine is a simple reductant with one-electron reducing ability, while L-NaAA is ionized in water to become L-NaAA monoanion, and this L-NaAA monoanion acts as a reductant in the reaction process. L-NaAA monoanion not only has one-electron reducing ability but also acts as a one-hydrogen donor, producing ESR-detectable L-NaAA radicals with a relatively long lifetime (Njus and Kelley, Biochim Biophys Acta 1993, 1144:235-48.). It was previously shown when employing quantum chemical calculations, in the photoreduction of IR700 in the presence of a one-electron reductant such as L-cysteine, axial ligand cleavage requires hydrolysis by a one-electron transfer from the reductant and a one-proton transfer from H₂O as occurs with L-NaAA (Kobayashi et al., Chempluschem 2020, 85:1959-1963). When L-cysteine was used as a reducing agent, one electron was donated to IR700, and long-lived IR700 anion radicals were detected by ESR because the proton transfer from H₂O to IR700 anion radical was relatively slow. However, when L-NaAA was used as the reducing agent, the donation of one electron and one proton from L-NaAA monoanion to IR700 molecules occurred simultaneously, so the IR700 anion radical proceeded quickly to the axial ligand cleavage reaction as soon as it was generated, and thus, could not be detected by ESR (FIGS. 17G and 30A).

Previous studies have indicated that NaN₃, a known singlet oxygen and ROS quencher, partially suppress the cytotoxicity of NIR-PIT. However, these experiments were performed in vitro because the necessary dose of NaN₃ is toxic for living animals in vivo. It is shown herein that NaN₃ directly suppressed the NIR light induced ligand release reaction of IR700 by quenching IR700 radical anion formation as shown on ESR (FIGS. 17G and 18F). Suppression of ligand release is measurable by IR700 fluorescence loss which also equates to treatment effectiveness. NaN₃ partially suppresses cytotoxicity by suppressing NIR light induced ligand release reaction of IR700. Therefore, the role of ROS in the mechanism of action of NIR-PIT cytotoxicity has been overstated in the literature (Kishimoto et al., Free radical biology & medicine 2015, 85:24-32; Railkar, et al., Molecular cancer therapeutics 2017, 16:2201-2214).

L-cysteine is an alternative to L-NaAA as a reducing agent due to its higher redox potential than L-NaAA and clinical use. In an IR700 solution, L-cysteine accelerated photo-induced ligand release by electron donation especially in hypoxic conditions. However, once the IR700 is conjugated to an antibody, NIR light induced ligand release reaction predominates under hypoxic conditions and is not highly influenced by L-cysteine. It is possible that the sulfhydryl groups on the L-cysteine side chains of antibodies could donate electron to IR700 when covalently conjugated and activated by the NIR light. L-cysteine addition even protected cells in vitro from NIR-PIT probably because L-cysteine form disulfide bonds with free sulfhydryl groups on the antibody under normoxic cell culture conditions. L-cystine formation hampers effective electron donation to light activated IR700 suppressing the ligand release reaction. L-NaAA is both an electron donor and can assist in protonation of excited IR700 which promotes the ligand release reaction. Therefore, although L-cysteine is a good reducing agent and might accelerate ligand release reaction in vitro, may be difficult to use for this purpose in vivo.

In summary, L-NaAA, acting as both a direct electron donor to IR700 and a reducing agent for ROS during NIR-PIT, facilitates selective cytotoxicity of NIR-PIT while suppressing local edema. Based on the relatively low toxicity of L-NaAA and safety clinical profile at the dose that used herein, it can serve as a useful adjuvant to NIR-PIT improving its safety profile while not interfering with its efficacy (FIG. 30B). Thus, one or more reducing agents, such as L-NaAA, can be used in combination with the disclosed methods, which utilize IR700. The one or more reducing agents in some examples significantly suppresses edema, for example soon after NIR-PIT. Clinically, edema is clinical problem with NIR-PIT. For example, when a tumor arising at the critical part of body (such as in the airways or mediastinum, such as behind the trachea) is treated, edema can cause undesirable events, such as chocking.

In view of the many possible embodiments to which the principles of the disclosure may be applied, it should be recognized that illustrated embodiments are only examples of the disclosure and should not be considered a limitation on the scope of the invention. Rather, the scope of the invention is defined by the following claims. We therefore claim as our invention all that comes within the scope and spirit of these claims.

1. A method for treating cancer in a subject, comprising:
(a) administering to the subject a therapeutically effective amount of
(i) one or more antibody-IR700 molecules, wherein the antibody specifically binds to a tumor-specific protein on the surface of a cancer cell, wherein the tumor-specific protein comprises epidermal growth factor receptor (EGFR/HER1), mesothelin, prostate specific membrane antigen (PSMA), HER2/ERBB2, CD3, CD18, CD20, CD25 (IL-2Rα receptor), CD30, CD33, CD44, CD52, CD133, CD206, carcinoembryonic antigen (CEA), alpha-fetoprotein (AFP), Lewis Y, tumor-associated glycoprotein 72 (TAG72), vascular endothelial growth factor (VEGF), VEGF receptor (VEGFR), epithelial cell adhesion molecule (EpCAM), ephrin type-A receptor 2 (EphA2), glypican-1, glypican-2, glypican-3, gpA33, a mucin, CAIX, a folate-binding protein, a ganglioside, integrin αVβ3, integrin α5 β3,1, Erb-B2 Receptor Tyrosine Kinase 3 (ERBB3), MET Proto-Oncogene, Receptor Tyrosine Kinase (MET), insulin like growth factor 1 receptor (IGF1R), ephrin type-A receptor 3 (EPHA3), tumor necrosis factor-related apoptosis-inducing ligand receptor 1 (TRAILR1), TRAILR2, receptor activator of nuclear factor kappa-B ligand (RANKL), fibroblast activation protein (FAP), tenascin, BCR complex, gp72, HLA-DR 10β, HLA-DR antigen, IgE, CA 242, polymorphic epithelial mucin (PEM) antigen, SK-1 antigen, programmed death 1 (PD-1), or programmed death ligand 2 (PD-L2); and/or
(ii) one or more immunoactivators;
(b) administering to the subject a therapeutically effective amount of one or more cytotoxic T-lymphocyte-associated protein 4 (CTLA4) antibody-IR700 molecules, one or more programmed death ligand 1 (PD-L1) antibody-IR700 molecules, or combinations thereof; and
(c) subsequently irradiating the subject and/or irradiating cancer cells in the subject at a wavelength of 660 to 740 nm and at a dose of at least 1 J/cm²;
wherein the one or more antibody-IR700 molecules, the one or more immunoactivators, the one or more CTLA4 antibody-IR700 molecules, and/or the one or more PD-L1 antibody-IR700 molecules, are administered sequentially or concurrently,
thereby treating the cancer in the subject.

2. The method of claim 1, wherein administering steps (a) and (b) comprise intravenous administration.

3. A method for treating cancer in a subject, comprising:
administering to the subject a therapeutically effective amount of one or more antibody-IR700 molecules, wherein the antibody specifically binds to a tumor-specific protein on the surface of a cancer cell or to an immune cell-specific protein on the surface of an immune cell;
administering to the subject a therapeutically effective amount of one or more reducing agents; and
irradiating the subject and/or irradiating cancer cells in the subject at a wavelength of 660 to 740 nm and at a dose of at least 1 J/cm²;
wherein the one or more antibody-IR700 molecules and the one or more reducing agents are administered sequentially or concurrently.

4. The method of claim 3, wherein the one or more antibody-IR700 molecules is administered intravenously and the one or more reducing agents is administered intraperitoneally.

5. The method of claim 3, wherein the irradiating is performed after both administering steps.

6. The method of claim 3, wherein the one or more reducing agents are administered prior to irradiating the subject and/or irradiating cancer cells in the subject.

7. The method of claim 3, wherein the tumor-specific protein comprises HER1/EGFR, mesothelin, PSMA, HER2/ERBB2, CD3, CD18, CD20, CD25 (IL-2Rα receptor), CD30, CD33, CD44, CD52, CD133, CD206, CEA, AFP, Lewis Y, TAG7), VEGF, VEGFR, EpCAM, EphA2, glypican-1, glypican-2, glypican-3, gpA33, a mucin, CAIX, a folate-binding protein, a ganglioside, integrin αVβ3, ERBB3, MET, IGF1R, EPHA3, TRAILR1, TRAILR2, RANKL, FAP, tenascin, BCR complex, gp72, HLA-DR 10β, HLA-DR antigen, IgE, CA 242, PEM antigen, SK-1 antigen, PD-1, or PD-L2, and the immune cell specific protein comprises CTLA4 or PD-L1.

8.-10. (canceled)

11. The method of claim 1, wherein the cancer or cancer cell is a cancer or cancer cell of the breast, liver, colon, ovary, prostate, pancreas, brain, cervix, kidney, bone, skin, head and neck, oropharynx or blood.

12. The method of claim 1, wherein
the CTLA4 antibody comprises ipilimumab or tremelimumab; and/or
the anti-PD-L1 antibody is atezolizumab, avelumab, durvalumab, cosibelimab, KN035 (envafolimab), BMS-936559, BMS935559, MEDI-4736, MPDL-3280A, or MEDI-4737.

13.-14. (canceled)

15. The method of claim 1, wherein the subject and/or the cancer cells are irradiated at a wavelength of 680 nm or 690 nm at a dose of 10 to 60 J/cm².

16. The method of claim 1, wherein the cancer cells are in a subject's blood, and wherein irradiating the cancer cells comprises irradiating the blood by using a device worn by the subject, wherein the device comprises a near infrared (NIR) light emitting diode (LED).

17. The method of claim 1, wherein the method further comprises:
selecting a subject with a cancer that expresses the tumor-specific protein that specifically binds to the antibody-IR700 molecule.

18. The method of claim 1, wherein the method reduces the weight, volume or size of the cancer by at least 25% relative to the absence of treatment;
reduces the weight, volume or size of a metastasis by at least 25% relative to the absence of treatment, wherein the metastasis is not irradiated at a wavelength of 660 to 740 nm and is located distant from the irradiated area of the tumor or lesion;
increases survival time of the subject relative to the absence of treatment;
increases progression-free survival time of the subject relative to the absence of treatment;
increases disease-free survival time of the subject relative to the absence of treatment; or
combinations thereof.

19. The method of claim 1, wherein the method
selectively kills CD4⁺Foxp3⁺ Tregs;
selectively depletes CTLA4⁺ cells from total live cell and T cell populations within the cancer but not within regional lymph nodes or the spleen;
increases the CD8⁺/CD4⁺Foxp3⁺ ratio;
increases the CD8+/Treg ratio;
increases the CD4⁺Foxp3⁻ cell:CD4⁺Foxp3⁺ cell ratio;
decreases intra-tumoral blood perfusion; or
combinations thereof.

20. The method of claim 3, wherein the method reduces edema and/or acute inflammatory reaction in the treated subject by at least 20% as compared to an amount of edema and/or acute inflammatory reaction in the absence of the one or more reducing agents.

21. The method of claim 3, wherein the cancer is a cancer in the airway or mediastinum.

22. The method of claim 3, wherein the one or more reducing agents comprise L-sodium ascorbate, ascorbic acid, L-cysteine, glutathione or combinations thereof.

23. (canceled)

24. The method of claim 3, further comprising administering to the subject a therapeutically effective amount of one or more CTLA4 antibody-IR700 molecules, one or more PD-L1 antibody-IR700 molecules, or combinations thereof.

25.-26. (canceled)

27. The method of claim 1, wherein the subject is administered two or more doses of the one or more one or more antibody-IR700 molecules that specifically bind to the tumor-specific protein on the surface of the cancer cell.

28. (canceled)

29. The method of claim 1, further comprising:
detecting the cancer cell with fluorescence lifetime imaging about 0 to 48 hours after the irradiating step.

30. The method of claim 1, wherein the subject is administered two or more doses of the one or more CTLA4 antibody-IR700 molecules, and/or the one or more PD-L1 antibody-IR700 molecules.

31. The method of claim 3, further comprising administering to the subject a therapeutically effective amount of one or more immunoactivators.

32.-33. (canceled)

34. A method for treating an EGFR-expressing cancer in a subject, comprising:
administering to the subject a therapeutically effective amount of one or more anti-EGFR-IR700 molecules;
administering to the subject a therapeutically effective amount of one or more anti-CTLA4-IR700 molecules; and
subsequently irradiating the subject and/or irradiating and EGFR-expressing cancer cells in the subject at a wavelength of 670 to 700 nm and at a dose of 4 to 100 J/cm²;
wherein the one or more anti-EGFR-IR700 molecules and the one or more anti-CTLA4-IR700 molecules are administered sequentially or concurrently,
thereby treating the EGFR-expressing cancer in the subject.

35. The method of claim 34, wherein the EGFR antibody comprises panitumumab or cetuximab.

36. (canceled)

37. A method for treating cancer in a subject, comprising:
administering to the subject a therapeutically effective amount of one or more anti-PD-L1-IR700 molecules;
administering to the subject a therapeutically effective amount of one or more immunoactivators; and
subsequently irradiating the subject and/or irradiating cancer cells in the subject at a wavelength of 670 to 700 nm and at a dose of 4 to 100 J/cm²;
wherein the one or more anti-PD-L1-IR700 molecules and the one or more immunoactivators are administered sequentially or concurrently,
thereby treating the cancer in the subject.

38.-39. (canceled)

40. The method of claim 37, further comprising administering to the subject a therapeutically effective amount of one or more antibody-IR700 molecules, wherein the antibody specifically binds to a tumor-specific protein on the surface of a cancer cell, wherein the tumor-specific protein comprises epidermal growth factor receptor HER1/EGFR, CTLA4, mesothelin, PSMA, HER2/ERBB2, CD3, CD18, CD20, CD25 (IL-2Rα receptor), CD30, CD33, CD44, CD52, CD133, CD206, CEA, AFP, Lewis Y, tumor-associated glycoprotein 72 (TAG72), VEGF, VEGFR, EpCAM, EphA2, glypican-3, gpA33, a mucin, CAIX, a folate-binding protein, a ganglioside, integrin αVβ3, ERBB3, MET, IGF1R, EPHA3, TRAILR1, TRAILR2, RANKL, FAP, tenascin, BCR complex, gp72, HLA-DR 10β, HLA-DR antigen, IgE, CA 242, PEM antigen, SK-1 antigen, PD-1, or PD-L2;

41. (canceled)

42. A method for reducing edema resulting from cancer treatment in a subject, comprising:
administering to the subject a therapeutically effective amount of one or more antibody-IR700 molecules, wherein the antibody specifically binds to a tumor-specific protein on the surface of a cancer cell or binds to a protein on the surface of an immune cell;
administering to the subject a therapeutically effective amount of L-sodium ascorbate; and
irradiating the subject and/or irradiating cancer cells in the subject at a wavelength of 660 to 740 nm and at a dose of at least 1 J/cm²;
wherein the one or more antibody-IR700 molecules and the one or more reducing agents are administered sequentially or concurrently,
thereby reducing edema resulting from cancer treatment in a subject.

43. The method of claim 42, wherein the protein on the surface of an immune cell is CTLA4 and the tumor-specific protein comprises HER1/EGFR, PD-L1, mesothelin, PSMA, HER2/ERBB2, CD3, CD18, CD20, CD25 (IL-2Rα receptor), CD30, CD33, CD44, CD52, CD133, CD206, CEA, AFP, Lewis Y, tumor-associated glycoprotein 72 (TAG72), VEGF, VEGFR, EpCAM, EphA2, glypican-3, gpA33, a mucin, CAIX, a folate-binding protein, a ganglioside, integrin αVβ3, ERBB3, MET, IGF1R, EPHA3, TRAILR1, TRAILR2, RANKL, FAP, tenascin, BCR complex, gp72, HLA-DR 10β, HLA-DR antigen, IgE, CA 242, PEM antigen, SK-1 antigen, PD-1, or PD-L2.

44. The method of claim 1, wherein the cancer is a highly or moderately immunogenic cancer.

45. (canceled)

46. The method of claim 1, wherein the cancer is a low immunogenic cancer.

47. (canceled)

48. The method of claim 1, wherein the method has an abscopal effect.
